SEQUENCE LISTING (571 sequences)

SEQ ID NO: 1 - protein, 743 aa, Homo sapiens
  SIGNAL 1..23
MEAVAVAAAVGVLLLAGAGGAAGDEAREAAAVRALVARLLGPGPAADFSVSVERALAAKPGLDTYSLGGGGAARVRVRGSTGVAAAAGLHRYLRDFCGCHVAWSGSQLRLPRPLPAVPGELTEATPNRYRYYQNVCTQSYSFVWWDWARWEREIDWMALNGINLALAWSGQEAIWQRVYLALGLTQAEINEFFTGPAFLAWGRMGNLHTWDGPLPPSWHIKQLYLQHRVLDQMRSFGMTPVLPAFAGHVPEAVTRVFPQVNVTKMGSWGHFNCSYSCSFLLAPEDPIFPIIGSLFLRELIKEFGTDHIYGADTFNEMQPPSSEPSYLAAATTAVYEAMTAVDTEAVWLLQGWLFQHQPQFWGPAQIRAVLGAVPRGRLLVLDLFAESQPVYTRTASFQGQPFIWCMLHNFGGNHGLFGALEAVNGGPEAARLFPNSTMVGTGMAPEGISQNEVVYSLMAELGWRKDPVPDLAAWVTSFAARRYGVSHPDAGAAWRLLLRSVYNCSGEACRGHNRSPLVRRPSLQMNTSIWYNRSDVFEAWRLLLTSAPSLATSPAFRYDLLDLTRQAVQELVSLYYEEARSAYLSKELASLLRAGGVLAYELLPALDEVLASDSRFLLGSWLEQARAAAVSEAEADFYEQNSRYQLTLWGPEGNILDYANKQLAGLVANYYTPRWRLFLEALVDSVAQGIPFQQHQFDKNVFQLEQAFVLSKQRYPSQPRGDTVDLAKKIFLKYYPRWVAGSW

SEQ ID NO: 2 - protein, 60 aa, Homo sapiens
LCGGELVDTLQFVCGDRGFYFSRPASRVSARSRGIVEECCFRSCDLALLETYCATPAKSE

SEQ ID NO: 3 - DNA, 2298 bp, Homo sapiens
  sig_peptide 1..69
  source 1..2298: mol_type = genomic DNA
atggaggcggtggcggtggccgcggcggtgggggtccttctcctggccggggccgggggcgcggcaggcatggaggcggtggcggtggccgcggcggtgggggtccttctcctggccggggccgggggcgcggcaggcgacgaggcccgggaggcggcggccgtgcgggcgctcgtggcccggctgctggggccaggccccgcggccgacttctccgtgtcggtggagcgcgctctggctgccaagccgggcttggacacctacagcctgggcggcggcggcgcggcgcgcgtgcgggtgcgcggctccacgggcgtggcggccgccgcggggctgcaccgctacctgcgcgacttctgtggctgccacgtggcctggtccggctctcagctgcgcctgccgcggccactgccagccgtgccgggggagctgaccgaggccacgcccaacaggtaccgctattaccagaatgtgtgcacgcaaagctactctttcgtgtggtgggactgggcccgctgggagcgagagatagactggatggcgctgaatggcatcaacctggcactggcctggagcggccaggaggccatctggcagcgggtgtacctggccttgggcctgacccaggcagagatcaatgagttctttactggtcctgccttcctggcctgggggcgaatgggcaacctgcacacctgggatggccccctgcccccctcctggcacatcaagcagctttacctgcagcaccgggtcctggaccagatgcgctccttcggcatgaccccagtgctgcctgcattcgcggggcatgttcccgaggctgtcaccagggtgttccctcaggtcaatgtcacgaagatgggcagttggggccactttaactgttcctactcctgctccttccttctggctccggaagaccccatattccccatcatcgggagcctcttcctgcgagagctgatcaaagagtttggcacagaccacatctatggggccgacactttcaatgagatgcagccaccttcctcagagccctcctaccttgccgcagccaccactgccgtctatgaggccatgactgcagtggatactgaggctgtgtggctgctccaaggctggctcttccagcaccagccgcagttctgggggcccgcccagatcagggctgtgctgggagctgtgccccgtggccgcctcctggttctggacctgtttgctgagagccagcctgtgtatacccgcactgcctccttccagggccagcccttcatctggtgcatgctgcacaactttgggggaaaccatggtctttttggagccctagaggctgtgaacggaggcccagaagctgcccgcctcttccccaactccaccatggtaggcacgggcatggcccccgagggcatcagccagaacgaagtggtctattccctcatggctgagctgggctggcgaaaggacccagtgccagatttggcagcctgggtgaccagctttgccgcccggcggtatggggtctcccacccggacgcaggggcagcgtggaggctactgctccggagtgtgtacaactgctccggggaggcctgcaggggccacaatcgtagcccgctggtcaggcggccgtccctacagatgaataccagcatctggtacaaccgatctgatgtgtttgaggcctggcggctgctgctcacatctgctccctccctggccaccagccccgccttccgctacgacctgctggacctcactcggcaggcagtgcaggagctggtcagcttgtactatgaggaggcaagaagcgcctacctgagcaaggagctggcctccctgttgagggctggaggcgtcctggcctatgagctgctgccggcactggacgaggtgctggctagtgacagccgcttcttgctgggcagctggctagagcaggcccgagcagcggcagtcagtgaggccgaggccgatttctacgagcagaacagccgctaccagctgaccttgtgggggccagaaggcaacatcctggactatgccaacaagcagctggcggggttggtggccaactactacacccctcgctggcggcttttcctggaggcgctggttgacagtgtggcccagggcatccctttccaacagcaccagtttgacaaaaatgtcttccaactggagcaggccttcgttctcagcaagcagaggtaccccagccagccgcgaggagacactgtggacctggccaagaagatcttcctcaaatattacccccgctgggtggccggctcttgg

SEQ ID NO: 4 - DNA, 180 bp, Homo sapiens
  source 1..180: mol_type = genomic DNA
ctgtgcggcggggagctggtggacaccctccagttcgtctgtggggaccgcggcttctacttcagcaggcccgcaagccgtgtgagcgctcgcagccgtggcatcgttgaggagtgctgtttccgcagctgtgacctggccctcctggagacgtactgtgctacccccgccaagtccgag

SEQ ID NO: 5 - protein, 67 aa, Homo sapiens
AYRPSETLCGGELVDTLQFVCGDRGFYFSRPASRVSRRSRGIVEECCFRSCDLALLETYCATPAKSE

SEQ ID NO: 6-7 - skipped (2 entries)

SEQ ID NO: 8 - protein, 17 aa, synthetic construct
  REGION 1..17: note = Synthetic peptide
MRAWIFFLLCLAGRALA

SEQ ID NO: 9-11 - skipped (3 entries)

SEQ ID NO: 12 - protein, 5 aa, synthetic construct
  REGION 1..5: note = Synthetic peptide
GGGGS

SEQ ID NO: 13 - protein, 6 aa, synthetic construct
  REGION 1..6: note = Synthetic peptide
GGGGGP

SEQ ID NO: 14 - protein, 5 aa, synthetic construct
  REGION 1..5: note = Synthetic peptide
GGGPS

SEQ ID NO: 15 - protein, 5 aa, synthetic construct
  REGION 1..5: note = Synthetic peptide
GGGSP

SEQ ID NO: 16 - protein, 4 aa, synthetic construct
  REGION 1..4: note = Synthetic peptide
GGGS

SEQ ID NO: 17 - protein, 5 aa, synthetic construct
  REGION 1..5: note = Synthetic peptide
AAAAS

SEQ ID NO: 18 - protein, 4 aa, synthetic construct
  REGION 1..4: note = Synthetic peptide
AAAS

SEQ ID NO: 19 - protein, 4 aa, synthetic construct
  REGION 1..4: note = Synthetic peptide
PAPA

SEQ ID NO: 20 - protein, 5 aa, synthetic construct
  REGION 1..5: note = Synthetic peptide
TPAPA

SEQ ID NO: 21 - protein, 5 aa, synthetic construct
  REGION 1..5: note = Synthetic peptide
AAAKE

SEQ ID NO: 22 - protein, 9 aa, synthetic construct
  REGION 1..9: note = Synthetic peptide
EFGGGGSTR

SEQ ID NO: 23 - protein, 7 aa, synthetic construct
  REGION 1..7: note = Synthetic peptide
GPSGSPG

SEQ ID NO: 24 - protein, 8 aa, synthetic construct
  REGION 1..8: note = Synthetic peptide
GPSGSPGT

SEQ ID NO: 25 - protein, 8 aa, synthetic construct
  REGION 1..8: note = Synthetic peptide
GPSGSPGH

SEQ ID NO: 26 - protein, 26 aa, synthetic construct
  REGION 1..26: note = Synthetic peptide
GGGGSGGGGSGGGGSGGGGSGGGPST

SEQ ID NO: 27 - protein, 26 aa, synthetic construct
  REGION 1..26: note = Synthetic peptide
GGGGSGGGGSGGGGSGGGGSGGGPSH

SEQ ID NO: 28 - protein, 35 aa, synthetic construct
  REGION 1..35: note = Synthetic peptide
GGGGSGGGGSGGGGSGGGGSGGGPSGGGGSGGGPS

SEQ ID NO: 29 - protein, 41 aa, synthetic construct
  REGION 1..41: note = Synthetic peptide
GAPGGGGSGGGGSGGGGSGGGGSGGGPSGGGGSGGGPSGAP

SEQ ID NO: 30 - protein, 50 aa, synthetic construct
  REGION 1..50: note = Synthetic peptide
GGGGSGGGGSGGGGSGGGGSGGGPSGGGGSGGGGSGGGGSGGGGSGGGPS

SEQ ID NO: 31 - protein, 56 aa, synthetic construct
  REGION 1..56: note = Synthetic peptide
GAPGGGGSGGGGSGGGGSGGGGSGGGPSGGGGSGGGGSGGGGSGGGGSGGGPSGAP

SEQ ID NO: 32 - protein, 35 aa, synthetic construct
  REGION 1..35: note = Synthetic peptide
GGGGSGGGGSGGGGSGGGPSGGGGSGGGGSGGGPS

SEQ ID NO: 33 - protein, 41 aa, synthetic construct
  REGION 1..41: note = Synthetic peptide
GAPGGGGSGGGGSGGGGSGGGPSGGGGSGGGGSGGGPSGAP

SEQ ID NO: 34 - protein, 50 aa, synthetic construct
  REGION 1..50: note = Synthetic peptide
GGGGSGGGGSGGGGSGGGPSGGGGSGGGGSGGGPSGGGGSGGGGSGGGPS

SEQ ID NO: 35 - protein, 56 aa, synthetic construct
  REGION 1..56: note = Synthetic peptide
GAPGGGGSGGGGSGGGGSGGGPSGGGGSGGGGSGGGPSGGGGSGGGGSGGGPSGAP

SEQ ID NO: 36 - protein, 25 aa, synthetic construct
  REGION 1..25: note = Synthetic peptide
GGGGSGGGGSGGGGSGGGGSGGGPS

SEQ ID NO: 37 - protein, 31 aa, synthetic construct
  REGION 1..31: note = Synthetic peptide
GAPGGGGSGGGGSGGGGSGGGGSGGGPSGAP

SEQ ID NO: 38 - protein, 25 aa, synthetic construct
  REGION 1..25: note = Synthetic peptide
GGGGSGGGGSAAAASGGGGSGGGPS

SEQ ID NO: 39 - protein, 31 aa, synthetic construct
  REGION 1..31: note = Synthetic peptide
GAPGGGGSGGGGSAAAASGGGGSGGGPSGAP

SEQ ID NO: 40 - protein, 50 aa, synthetic construct
  REGION 1..50: note = Synthetic peptide
GGGGSGGGGSAAAASGGGGSGGGGSAAAASGGGGSGGGGSAAAASGGGPS

SEQ ID NO: 41 - protein, 56 aa, synthetic construct
  REGION 1..56: note = Synthetic peptide
GAPGGGGSGGGGSAAAASGGGGSGGGGSAAAASGGGGSGGGGSAAAASGGGPSGAP

SEQ ID NO: 42 - protein, 50 aa, synthetic construct
  REGION 1..50: note = Synthetic peptide
GGGGSGGGGSAAAASGGGPSGGGGSAAAASGGGPSGGGGSAAAASGGGPS

SEQ ID NO: 43 - protein, 56 aa, synthetic construct
  REGION 1..56: note = Synthetic peptide
GAPGGGGSGGGGSAAAASGGGPSGGGGSAAAASGGGPSGGGGSAAAASGGGPSGAP

SEQ ID NO: 44 - protein, 47 aa, synthetic construct
  REGION 1..47: note = Synthetic peptide
GGSPAGSPTSTEEGTSESATPESGPGTSTEPSEGSAPGSPAGSPGPS

SEQ ID NO: 45 - protein, 53 aa, synthetic construct
  REGION 1..53: note = Synthetic peptide
GAPGGSPAGSPTSTEEGTSESATPESGPGTSTEPSEGSAPGSPAGSPGPSGAP

SEQ ID NO: 46 - protein, 50 aa, synthetic construct
  REGION 1..50: note = Synthetic peptide
GGSPAGSPTSTEEGTSESATPESGPGTSTEPSEGSAPGSPAGSPTSTGPS

SEQ ID NO: 47 - protein, 56 aa, synthetic construct
  REGION 1..56: note = Synthetic peptide
GAPGGSPAGSPTSTEEGTSESATPESGPGTSTEPSEGSAPGSPAGSPTSTGPSGAP

SEQ ID NO: 48 - protein, 25 aa, synthetic construct
  REGION 1..25: note = Synthetic peptide
GGGSPAPTPTPAPTPAPTPAGGGPS

SEQ ID NO: 49 - protein, 31 aa, synthetic construct
  REGION 1..31: note = Synthetic peptide
GAPGGGSPAPTPTPAPTPAPTPAGGGPSGAP

SEQ ID NO: 50 - protein, 25 aa, synthetic construct
  REGION 1..25: note = Synthetic peptide
GGGSPAPAPTPAPAPTPAPAGGGPS

SEQ ID NO: 51 - protein, 31 aa, synthetic construct
  REGION 1..31: note = Synthetic peptide
GAPGGGSPAPAPTPAPAPTPAPAGGGPSGAP

SEQ ID NO: 52 - protein, 25 aa, synthetic construct
  REGION 1..25: note = Synthetic peptide
GGGSAEAAAKEAAAKEAAAKAGGPS

SEQ ID NO: 53 - protein, 31 aa, synthetic construct
  REGION 1..31: note = Synthetic peptide
GAPGGGSAEAAAKEAAAKEAAAKAGGPSGAP

SEQ ID NO: 54 - protein, 37 aa, synthetic construct
  REGION 1..37: note = Synthetic peptide
GGGSPAEAAAKEAAAKEAAAKEAAAKEAAAKAPSGGG

SEQ ID NO: 55 - protein, 43 aa, synthetic construct
  REGION 1..43: note = Synthetic peptide
GAPGGGSPAEAAAKEAAAKEAAAKEAAAKEAAAKAPSGGGGAP

SEQ ID NO: 56 - protein, 15 aa, synthetic construct
  REGION 1..15: note = Synthetic peptide
GGGGSGGGGSGGGGS

SEQ ID NO: 57 - protein, 21 aa, synthetic construct
  REGION 1..21: note = Synthetic peptide
GAPGGGGSGGGGSGGGGSGAP

SEQ ID NO: 58 - protein, 20 aa, synthetic construct
  REGION 1..20: note = Synthetic peptide
GGGGSGGGGSGGGGSGGGGS

SEQ ID NO: 59 - protein, 26 aa, synthetic construct
  REGION 1..26: note = Synthetic peptide
GAPGGGGSGGGGSGGGGSGGGGSGAP

SEQ ID NO: 60 - protein, 5 aa, synthetic construct
  REGION 1..5: note = Synthetic peptide
GGGGA

SEQ ID NO: 61 - protein, 26 aa, synthetic construct
  REGION 1..26: note = Synthetic peptide
GGGGAGGGGAGGGGAGGGGAGGGPST

SEQ ID NO: 62 - protein, 26 aa, synthetic construct
  REGION 1..26: note = Synthetic peptide
GGGGAGGGGAGGGGAGGGGAGGGPSH

SEQ ID NO: 63 - protein, 35 aa, synthetic construct
  REGION 1..35: note = Synthetic peptide
GGGGAGGGGAGGGGAGGGGAGGGPSGGGGAGGGPS

SEQ ID NO: 64 - protein, 41 aa, synthetic construct
  REGION 1..41: note = Synthetic peptide
GAPGGGGAGGGGAGGGGAGGGGAGGGPSGGGGAGGGPSGAP

SEQ ID NO: 65 - protein, 50 aa, synthetic construct
  REGION 1..50: note = Synthetic peptide
GGGGAGGGGAGGGGAGGGGAGGGPSGGGGAGGGGAGGGGAGGGGAGGGPS

SEQ ID NO: 66 - protein, 56 aa, synthetic construct
  REGION 1..56: note = Synthetic peptide
GAPGGGGAGGGGAGGGGAGGGGAGGGPSGGGGAGGGGAGGGGAGGGGAGGGPSGAP

SEQ ID NO: 67 - protein, 35 aa, synthetic construct
  REGION 1..35: note = Synthetic peptide
GGGGAGGGGAGGGGAGGGPSGGGGAGGGGAGGGPS

SEQ ID NO: 68 - protein, 41 aa, synthetic construct
  REGION 1..41: note = Synthetic peptide
GAPGGGGAGGGGAGGGGAGGGPSGGGGAGGGGAGGGPSGAP

SEQ ID NO: 69 - protein, 50 aa, synthetic construct
  REGION 1..50: note = Synthetic peptide
GGGGAGGGGAGGGGAGGGPSGGGGAGGGGAGGGPSGGGGAGGGGAGGGPS

SEQ ID NO: 70 - protein, 56 aa, synthetic construct
  REGION 1..56: note = Synthetic peptide
GAPGGGGAGGGGAGGGGAGGGPSGGGGAGGGGAGGGPSGGGGAGGGGAGGGPSGAP

SEQ ID NO: 71 - protein, 25 aa, synthetic construct
  REGION 1..25: note = Synthetic peptide
GGGGAGGGGAGGGGAGGGGAGGGPS

SEQ ID NO: 72 - protein, 31 aa, synthetic construct
  REGION 1..31: note = Synthetic peptide
GAPGGGGAGGGGAGGGGAGGGGAGGGPSGAP

SEQ ID NO: 73 - protein, 25 aa, synthetic construct
  REGION 1..25: note = Synthetic peptide
GGGGAGGGGAAAAASGGGGAGGGPS

SEQ ID NO: 74 - protein, 31 aa, synthetic construct
  REGION 1..31: note = Synthetic peptide
GAPGGGGAGGGGAAAAASGGGGAGGGPSGAP

SEQ ID NO: 75 - protein, 50 aa, synthetic construct
  REGION 1..50: note = Synthetic peptide
GGGGAGGGGAAAAASGGGGAGGGGAAAAASGGGGAGGGGAAAAASGGGPS

SEQ ID NO: 76 - protein, 56 aa, synthetic construct
  REGION 1..56: note = Synthetic peptide
GAPGGGGAGGGGAAAAASGGGGAGGGGAAAAASGGGGAGGGGAAAAASGGGPSGAP

SEQ ID NO: 77 - protein, 50 aa, synthetic construct
  REGION 1..50: note = Synthetic peptide
GGGGAGGGGAAAAASGGGPSGGGGAAAAASGGGPSGGGGAAAAASGGGPS

SEQ ID NO: 78 - protein, 56 aa, synthetic construct
  REGION 1..56: note = Synthetic peptide
GAPGGGGAGGGGAAAAASGGGPSGGGGAAAAASGGGPSGGGGAAAAASGGGPSGAP

SEQ ID NO: 79 - protein, 15 aa, synthetic construct
  REGION 1..15: note = Synthetic peptide
GGGGAGGGGAGGGGA

SEQ ID NO: 80 - protein, 21 aa, synthetic construct
  REGION 1..21: note = Synthetic peptide
GAPGGGGAGGGGAGGGGAGAP

SEQ ID NO: 81 - protein, 20 aa, synthetic construct
  REGION 1..20: note = Synthetic peptide
GGGGAGGGGAGGGGAGGGGA

SEQ ID NO: 82 - protein, 26 aa, synthetic construct
  REGION 1..26: note = Synthetic peptide
GAPGGGGAGGGGAGGGGAGGGGAGAP

SEQ ID NO: 83 - protein, 45 aa, synthetic construct
  REGION 1..45: note = Synthetic peptide
GGGGAGGGGAGGGGAGGGGAGGGGAGGGGAGGGGAGGGGAGGGPS

SEQ ID NO: 84 - protein, 46 aa, synthetic construct
  REGION 1..46: note = Synthetic peptide
GGGGAGGGGAGGGGAGGGGAGGGGAGGGGAGGGGAGGGGAGGGPSH

SEQ ID NO: 85 - protein, 50 aa, synthetic construct
  REGION 1..50: note = Synthetic peptide
GGGGAGGGGAGGGGAGGGGAGGGGAGGGGAGGGGAGGGGAGGGGAGGGPS

SEQ ID NO: 86 - protein, 51 aa, synthetic construct
  REGION 1..51: note = Synthetic peptide
GGGGAGGGGAGGGGAGGGGAGGGGAGGGGAGGGGAGGGGAGGGGAGGGPSH

SEQ ID NO: 87 - protein, 25 aa, synthetic construct
  REGION 1..25: note = Synthetic peptide
GGGGPAPGPGPAPGPAPGPAGGGPS

SEQ ID NO: 88 - protein, 31 aa, synthetic construct
  REGION 1..31: note = Synthetic peptide
GAPGGGGPAPGPGPAPGPAPGPAGGGPGGAP

SEQ ID NO: 89 - protein, 25 aa, synthetic construct
  REGION 1..25: note = Synthetic peptide
GGGGPAPAPGPAPAPGPAPAGGGPS

SEQ ID NO: 90 - protein, 31 aa, synthetic construct
  REGION 1..31: note = Synthetic peptide
GAPGGGGPAPAPGPAPAPGPAPAGGGPGGAP

SEQ ID NO: 91 - protein, 10 aa, synthetic construct
  REGION 1..10: note = Synthetic peptide
GGGGSGGGGS

SEQ ID NO: 92 - protein, 25 aa, synthetic construct
  REGION 1..25: note = Synthetic peptide
GGGGSGGGGSGGGGSGGGGSGGGGS

SEQ ID NO: 93 - protein, 30 aa, synthetic construct
  REGION 1..30: note = Synthetic peptide
GGGGSGGGGSGGGGSGGGGSGGGGSGGGGS

SEQ ID NO: 94 - protein, 35 aa, synthetic construct
  REGION 1..35: note = Synthetic peptide
GGGGSGGGGSGGGGSGGGGSGGGGSGGGGSGGGGS

SEQ ID NO: 95 - protein, 10 aa, synthetic construct
  REGION 1..10: note = Synthetic peptide
GGGGSGGGPS

SEQ ID NO: 96 - protein, 15 aa, synthetic construct
  REGION 1..15: note = Synthetic peptide
GGGGSGGGGSGGGPS

SEQ ID NO: 97 - protein, 20 aa, synthetic construct
  REGION 1..20: note = Synthetic peptide
GGGGSGGGGSGGGGSGGGPS

SEQ ID NO: 98 - protein, 30 aa, synthetic construct
  REGION 1..30: note = Synthetic peptide
GGGGSGGGGSGGGGSGGGGSGGGGSGGGPS

SEQ ID NO: 99 - protein, 35 aa, synthetic construct
  REGION 1..35: note = Synthetic peptide
GGGGSGGGGSGGGGSGGGGSGGGGSGGGGSGGGPS

SEQ ID NO: 100 - protein, 40 aa, synthetic construct
  REGION 1..40: note = Synthetic peptide
GGGGSGGGGSGGGGSGGGGSGGGGSGGGGSGGGGSGGGPS

SEQ ID NO: 101 - protein, 13 aa, synthetic construct
  REGION 1..13: note = Synthetic peptide
GAPGGGGSGGGPS

SEQ ID NO: 102 - protein, 18 aa, synthetic construct
  REGION 1..18: note = Synthetic peptide
GAPGGGGSGGGGSGGGPS

SEQ ID NO: 103 - protein, 23 aa, synthetic construct
  REGION 1..23: note = Synthetic peptide
GAPGGGGSGGGGSGGGGSGGGPS

SEQ ID NO: 104 - protein, 28 aa, synthetic construct
  REGION 1..28: note = Synthetic peptide
GAPGGGGSGGGGSGGGGSGGGGSGGGPS

SEQ ID NO: 105 - protein, 33 aa, synthetic construct
  REGION 1..33: note = Synthetic peptide
GAPGGGGSGGGGSGGGGSGGGGSGGGGSGGGPS

SEQ ID NO: 106 - protein, 38 aa, synthetic construct
  REGION 1..38: note = Synthetic peptide
GAPGGGGSGGGGSGGGGSGGGGSGGGGSGGGGSGGGPS

SEQ ID NO: 107 - protein, 43 aa, synthetic construct
  REGION 1..43: note = Synthetic peptide
GAPGGGGSGGGGSGGGGSGGGGSGGGGSGGGGSGGGGSGGGPS

SEQ ID NO: 108 - protein, 16 aa, synthetic construct
  REGION 1..16: note = Synthetic peptide
GAPGGGGSGGGPSGAP

SEQ ID NO: 109 - protein, 21 aa, synthetic construct
  REGION 1..21: note = Synthetic peptide
GAPGGGGSGGGGSGGGPSGAP

SEQ ID NO: 110 - protein, 26 aa, synthetic construct
  REGION 1..26: note = Synthetic peptide
GAPGGGGSGGGGSGGGGSGGGPSGAP

SEQ ID NO: 111 - protein, 36 aa, synthetic construct
  REGION 1..36: note = Synthetic peptide
GAPGGGGSGGGGSGGGGSGGGGSGGGGSGGGPSGAP

SEQ ID NO: 112 - protein, 41 aa, synthetic construct
  REGION 1..41: note = Synthetic peptide
GAPGGGGSGGGGSGGGGSGGGGSGGGGSGGGGSGGGPSGAP

SEQ ID NO: 113 - protein, 46 aa, synthetic construct
  REGION 1..46: note = Synthetic peptide
GAPGGGGSGGGGSGGGGSGGGGSGGGGSGGGGSGGGGSGGGPSGAP

SEQ ID NO: 114 - protein, 21 aa, synthetic construct
  REGION 1..21: note = Synthetic peptide
GAPGGGGSGGGPSGGGGSGAP

SEQ ID NO: 115 - protein, 26 aa, synthetic construct
  REGION 1..26: note = Synthetic peptide
GAPGGGGSGGGPSGGGGSGGGGSGAP

SEQ ID NO: 116 - protein, 31 aa, synthetic construct
  REGION 1..31: note = Synthetic peptide
GAPGGGGSGGGPSGGGGSGGGGSGGGGSGAP

SEQ ID NO: 117 - protein, 36 aa, synthetic construct
  REGION 1..36: note = Synthetic peptide
GAPGGGGSGGGPSGGGGSGGGGSGGGGSGGGGSGAP

SEQ ID NO: 118 - protein, 41 aa, synthetic construct
  REGION 1..41: note = Synthetic peptide
GAPGGGGSGGGPSGGGGSGGGGSGGGGSGGGGSGGGGSGAP

SEQ ID NO: 119 - protein, 46 aa, synthetic construct
  REGION 1..46: note = Synthetic peptide
GAPGGGGSGGGPSGGGGSGGGGSGGGGSGGGGSGGGGSGGGGSGAP

SEQ ID NO: 120 - protein, 51 aa, synthetic construct
  REGION 1..51: note = Synthetic peptide
GAPGGGGSGGGPSGGGGSGGGGSGGGGSGGGGSGGGGSGGGGSGGGGSGAP

SEQ ID NO: 121 - protein, 26 aa, synthetic construct
  REGION 1..26: note = Synthetic peptide
GAPGGGGSGGGGSGGGPSGGGGSGAP

SEQ ID NO: 122 - protein, 31 aa, synthetic construct
  REGION 1..31: note = Synthetic peptide
GAPGGGGSGGGGSGGGPSGGGGSGGGGSGAP

SEQ ID NO: 123 - protein, 36 aa, synthetic construct
  REGION 1..36: note = Synthetic peptide
GAPGGGGSGGGGSGGGPSGGGGSGGGGSGGGGSGAP

SEQ ID NO: 124 - protein, 41 aa, synthetic construct
  REGION 1..41: note = Synthetic peptide
GAPGGGGSGGGGSGGGPSGGGGSGGGGSGGGGSGGGGSGAP

SEQ ID NO: 125 - protein, 46 aa, synthetic construct
  REGION 1..46: note = Synthetic peptide
GAPGGGGSGGGGSGGGPSGGGGSGGGGSGGGGSGGGGSGGGGSGAP

SEQ ID NO: 126 - protein, 51 aa, synthetic construct
  REGION 1..51: note = Synthetic peptide
GAPGGGGSGGGGSGGGPSGGGGSGGGGSGGGGSGGGGSGGGGSGGGGSGAP

SEQ ID NO: 127 - protein, 56 aa, synthetic construct
  REGION 1..56: note = Synthetic peptide
GAPGGGGSGGGGSGGGPSGGGGSGGGGSGGGGSGGGGSGGGGSGGGGSGGGGSGAP

SEQ ID NO: 128 - protein, 31 aa, synthetic construct
  REGION 1..31: note = Synthetic peptide
GAPGGGGSGGGGSGGGGSGGGPSGGGGSGAP

SEQ ID NO: 129 - protein, 36 aa, synthetic construct
  REGION 1..36: note = Synthetic peptide
GAPGGGGSGGGGSGGGGSGGGPSGGGGSGGGGSGAP

SEQ ID NO: 130 - protein, 41 aa, synthetic construct
  REGION 1..41: note = Synthetic peptide
GAPGGGGSGGGGSGGGGSGGGPSGGGGSGGGGSGGGGSGAP

SEQ ID NO: 131 - protein, 46 aa, synthetic construct
  REGION 1..46: note = Synthetic peptide
GAPGGGGSGGGGSGGGGSGGGPSGGGGSGGGGSGGGGSGGGGSGAP

SEQ ID NO: 132 - protein, 51 aa, synthetic construct
  REGION 1..51: note = Synthetic peptide
GAPGGGGSGGGGSGGGGSGGGPSGGGGSGGGGSGGGGSGGGGSGGGGSGAP

SEQ ID NO: 133 - protein, 56 aa, synthetic construct
  REGION 1..56: note = Synthetic peptide
GAPGGGGSGGGGSGGGGSGGGPSGGGGSGGGGSGGGGSGGGGSGGGGSGGGGSGAP

SEQ ID NO: 134 - protein, 61 aa, synthetic construct
  REGION 1..61: note = Synthetic peptide
GAPGGGGSGGGGSGGGGSGGGPSGGGGSGGGGSGGGGSGGGGSGGGGSGGGGSGGGGSGAP

SEQ ID NO: 135 - protein, 36 aa, synthetic construct
  REGION 1..36: note = Synthetic peptide
GAPGGGGSGGGGSGGGGSGGGGSGGGPSGGGGSGAP

SEQ ID NO: 136 - protein, 41 aa, synthetic construct
  REGION 1..41: note = Synthetic peptide
GAPGGGGSGGGGSGGGGSGGGGSGGGPSGGGGSGGGGSGAP

SEQ ID NO: 137 - protein, 46 aa, synthetic construct
  REGION 1..46: note = Synthetic peptide
GAPGGGGSGGGGSGGGGSGGGGSGGGPSGGGGSGGGGSGGGGSGAP

SEQ ID NO: 138 - protein, 51 aa, synthetic construct
  REGION 1..51: note = Synthetic peptide
GAPGGGGSGGGGSGGGGSGGGGSGGGPSGGGGSGGGGSGGGGSGGGGSGAP

SEQ ID NO: 139 - protein, 56 aa, synthetic construct
  REGION 1..56: note = Synthetic peptide
GAPGGGGSGGGGSGGGGSGGGGSGGGPSGGGGSGGGGSGGGGSGGGGSGGGGSGAP

SEQ ID NO: 140 - protein, 61 aa, synthetic construct
  REGION 1..61: note = Synthetic peptide
GAPGGGGSGGGGSGGGGSGGGGSGGGPSGGGGSGGGGSGGGGSGGGGSGGGGSGGGGSGAP

SEQ ID NO: 141 - protein, 66 aa, synthetic construct
  REGION 1..66: note = Synthetic peptide
GAPGGGGSGGGGSGGGGSGGGGSGGGPSGGGGSGGGGSGGGGSGGGGSGGGGSGGGGSGGGGSGAP

SEQ ID NO: 142 - protein, 41 aa, synthetic construct
  REGION 1..41: note = Synthetic peptide
GAPGGGGSGGGGSGGGGSGGGGSGGGGSGGGPSGGGGSGAP

SEQ ID NO: 143 - protein, 46 aa, synthetic construct
  REGION 1..46: note = Synthetic peptide
GAPGGGGSGGGGSGGGGSGGGGSGGGGSGGGPSGGGGSGGGGSGAP

SEQ ID NO: 144 - protein, 51 aa, synthetic construct
  REGION 1..51: note = Synthetic peptide
GAPGGGGSGGGGSGGGGSGGGGSGGGGSGGGPSGGGGSGGGGSGGGGSGAP

SEQ ID NO: 145 - protein, 56 aa, synthetic construct
  REGION 1..56: note = Synthetic peptide
GAPGGGGSGGGGSGGGGSGGGGSGGGGSGGGPSGGGGSGGGGSGGGGSGGGGSGAP

SEQ ID NO: 146 - protein, 61 aa, synthetic construct
  REGION 1..61: note = Synthetic peptide
GAPGGGGSGGGGSGGGGSGGGGSGGGGSGGGPSGGGGSGGGGSGGGGSGGGGSGGGGSGAP

SEQ ID NO: 147 - protein, 66 aa, synthetic construct
  REGION 1..66: note = Synthetic peptide
GAPGGGGSGGGGSGGGGSGGGGSGGGGSGGGPSGGGGSGGGGSGGGGSGGGGSGGGGSGGGGSGAP

SEQ ID NO: 148 - protein, 71 aa, synthetic construct
  REGION 1..71: note = Synthetic peptide
GAPGGGGSGGGGSGGGGSGGGGSGGGGSGGGPSGGGGSGGGGSGGGGSGGGGSGGGGSGGGGSGGGGSGAP

SEQ ID NO: 149 - protein, 46 aa, synthetic construct
  REGION 1..46: note = Synthetic peptide
GAPGGGGSGGGGSGGGGSGGGGSGGGGSGGGGSGGGPSGGGGSGAP

SEQ ID NO: 150 - protein, 51 aa, synthetic construct
  REGION 1..51: note = Synthetic peptide
GAPGGGGSGGGGSGGGGSGGGGSGGGGSGGGGSGGGPSGGGGSGGGGSGAP

SEQ ID NO: 151 - protein, 56 aa, synthetic construct
  REGION 1..56: note = Synthetic peptide
GAPGGGGSGGGGSGGGGSGGGGSGGGGSGGGGSGGGPSGGGGSGGGGSGGGGSGAP

SEQ ID NO: 152 - protein, 61 aa, synthetic construct
  REGION 1..61: note = Synthetic peptide
GAPGGGGSGGGGSGGGGSGGGGSGGGGSGGGGSGGGPSGGGGSGGGGSGGGGSGGGGSGAP

SEQ ID NO: 153 - protein, 66 aa, synthetic construct
  REGION 1..66: note = Synthetic peptide
GAPGGGGSGGGGSGGGGSGGGGSGGGGSGGGGSGGGPSGGGGSGGGGSGGGGSGGGGSGGGGSGAP

SEQ ID NO: 154 - protein, 71 aa, synthetic construct
  REGION 1..71: note = Synthetic peptide
GAPGGGGSGGGGSGGGGSGGGGSGGGGSGGGGSGGGPSGGGGSGGGGSGGGGSGGGGSGGGGSGGGGSGAP

SEQ ID NO: 155 - protein, 76 aa, synthetic construct
  REGION 1..76: note = Synthetic peptide
GAPGGGGSGGGGSGGGGSGGGGSGGGGSGGGGSGGGPSGGGGSGGGGSGGGGSGGGGSGGGGSGGGGSGGGGSGAP

SEQ ID NO: 156 - protein, 51 aa, synthetic construct
  REGION 1..51: note = Synthetic peptide
GAPGGGGSGGGGSGGGGSGGGGSGGGGSGGGGSGGGGSGGGPSGGGGSGAP

SEQ ID NO: 157 - protein, 56 aa, synthetic construct
  REGION 1..56: note = Synthetic peptide
GAPGGGGSGGGGSGGGGSGGGGSGGGGSGGGGSGGGGSGGGPSGGGGSGGGGSGAP

SEQ ID NO: 158 - protein, 61 aa, synthetic construct
  REGION 1..61: note = Synthetic peptide
GAPGGGGSGGGGSGGGGSGGGGSGGGGSGGGGSGGGGSGGGPSGGGGSGGGGSGGGGSGAP

SEQ ID NO: 159 - protein, 66 aa, synthetic construct
  REGION 1..66: note = Synthetic peptide
GAPGGGGSGGGGSGGGGSGGGGSGGGGSGGGGSGGGGSGGGPSGGGGSGGGGSGGGGSGGGGSGAP

SEQ ID NO: 160 - protein, 71 aa, synthetic construct
  REGION 1..71: note = Synthetic peptide
GAPGGGGSGGGGSGGGGSGGGGSGGGGSGGGGSGGGGSGGGPSGGGGSGGGGSGGGGSGGGGSGGGGSGAP

SEQ ID NO: 161 - protein, 76 aa, synthetic construct
  REGION 1..76: note = Synthetic peptide
GAPGGGGSGGGGSGGGGSGGGGSGGGGSGGGGSGGGGSGGGPSGGGGSGGGGSGGGGSGGGGSGGGGSGGGGSGAP

SEQ ID NO: 162 - protein, 81 aa, synthetic construct
  REGION 1..81: note = Synthetic peptide
GAPGGGGSGGGGSGGGGSGGGGSGGGGSGGGGSGGGGSGGGPSGGGGSGGGGSGGGGSGGGGSGGGGSGGGGSGGGGSGAP

SEQ ID NO: 163 - protein, 16 aa, synthetic construct
  REGION 1..16: note = Synthetic peptide
GAPGGGPSGGGGSGAP

SEQ ID NO: 164 - protein, 21 aa, synthetic construct
  REGION 1..21: note = Synthetic peptide
GAPGGGPSGGGGSGGGGSGAP

SEQ ID NO: 165 - protein, 26 aa, synthetic construct
  REGION 1..26: note = Synthetic peptide
GAPGGGPSGGGGSGGGGSGGGGSGAP

SEQ ID NO: 166 - protein, 31 aa, synthetic construct
  REGION 1..31: note = Synthetic peptide
GAPGGGPSGGGGSGGGGSGGGGSGGGGSGAP

SEQ ID NO: 167 - protein, 36 aa, synthetic construct
  REGION 1..36: note = Synthetic peptide
GAPGGGPSGGGGSGGGGSGGGGSGGGGSGGGGSGAP

SEQ ID NO: 168 - protein, 41 aa, synthetic construct
  REGION 1..41: note = Synthetic peptide
GAPGGGPSGGGGSGGGGSGGGGSGGGGSGGGGSGGGGSGAP

SEQ ID NO: 169 - protein, 46 aa, synthetic construct
  REGION 1..46: note = Synthetic peptide
GAPGGGPSGGGGSGGGGSGGGGSGGGGSGGGGSGGGGSGGGGSGAP

SEQ ID NO: 170 - protein, 30 aa, synthetic construct
  REGION 1..30: note = Synthetic peptide
GAPGGGGSAAAASGGGPSGGGGSAAAAGAP

SEQ ID NO: 171 - protein, 35 aa, synthetic construct
  REGION 1..35: note = Synthetic peptide
GAPGGGGSAAAASGGGPSGGGGSGGGGSAAAAGAP

SEQ ID NO: 172 - protein, 40 aa, synthetic construct
  REGION 1..40: note = Synthetic peptide
GAPGGGGSAAAASGGGPSGGGGSGGGGSGGGGSAAAAGAP

SEQ ID NO: 173 - protein, 45 aa, synthetic construct
  REGION 1..45: note = Synthetic peptide
GAPGGGGSAAAASGGGPSGGGGSGGGGSGGGGSGGGGSAAAAGAP

SEQ ID NO: 174 - protein, 50 aa, synthetic construct
  REGION 1..50: note = Synthetic peptide
GAPGGGGSAAAASGGGPSGGGGSGGGGSGGGGSGGGGSGGGGSAAAAGAP

SEQ ID NO: 175 - protein, 55 aa, synthetic construct
  REGION 1..55: note = Synthetic peptide
GAPGGGGSAAAASGGGPSGGGGSGGGGSGGGGSGGGGSGGGGSGGGGSAAAAGAP

SEQ ID NO: 176 - protein, 60 aa, synthetic construct
  REGION 1..60: note = Synthetic peptide
GAPGGGGSAAAASGGGPSGGGGSGGGGSGGGGSGGGGSGGGGSGGGGSGGGGSAAAAGAP

SEQ ID NO: 177 - protein, 35 aa, synthetic construct
  REGION 1..35: note = Synthetic peptide
GAPGGGGSGGGGSAAAASGGGPSGGGGSAAAAGAP

SEQ ID NO: 178 - protein, 40 aa, synthetic construct
  REGION 1..40: note = Synthetic peptide
GAPGGGGSGGGGSAAAASGGGPSGGGGSGGGGSAAAAGAP

SEQ ID NO: 179 - protein, 45 aa, synthetic construct
  REGION 1..45: note = Synthetic peptide
GAPGGGGSGGGGSAAAASGGGPSGGGGSGGGGSGGGGSAAAAGAP

SEQ ID NO: 180 - protein, 50 aa, synthetic construct
  REGION 1..50: note = Synthetic peptide
GAPGGGGSGGGGSAAAASGGGPSGGGGSGGGGSGGGGSGGGGSAAAAGAP

SEQ ID NO: 181 - protein, 55 aa, synthetic construct
  REGION 1..55: note = Synthetic peptide
GAPGGGGSGGGGSAAAASGGGPSGGGGSGGGGSGGGGSGGGGSGGGGSAAAAGAP

SEQ ID NO: 182 - protein, 60 aa, synthetic construct
  REGION 1..60: note = Synthetic peptide
GAPGGGGSGGGGSAAAASGGGPSGGGGSGGGGSGGGGSGGGGSGGGGSGGGGSAAAAGAP

SEQ ID NO: 183 - protein, 65 aa, synthetic construct
  REGION 1..65: note = Synthetic peptide
GAPGGGGSGGGGSAAAASGGGPSGGGGSGGGGSGGGGSGGGGSGGGGSGGGGSGGGGSAAAAGAP

SEQ ID NO: 184 - protein, 40 aa, synthetic construct
  REGION 1..40: note = Synthetic peptide
GAPGGGGSGGGGSGGGGSAAAASGGGPSGGGGSAAAAGAP

SEQ ID NO: 185 - protein, 45 aa, synthetic construct
  REGION 1..45: note = Synthetic peptide
GAPGGGGSGGGGSGGGGSAAAASGGGPSGGGGSGGGGSAAAAGAP

SEQ ID NO: 186 - protein, 50 aa, synthetic construct
  REGION 1..50: note = Synthetic peptide
GAPGGGGSGGGGSGGGGSAAAASGGGPSGGGGSGGGGSGGGGSAAAAGAP

SEQ ID NO: 187 - protein, 55 aa, synthetic construct
  REGION 1..55: note = Synthetic peptide
GAPGGGGSGGGGSGGGGSAAAASGGGPSGGGGSGGGGSGGGGSGGGGSAAAAGAP

SEQ ID NO: 188 - protein, 60 aa, synthetic construct
  REGION 1..60: note = Synthetic peptide
GAPGGGGSGGGGSGGGGSAAAASGGGPSGGGGSGGGGSGGGGSGGGGSGGGGSAAAAGAP

SEQ ID NO: 189 - protein, 65 aa, synthetic construct
  REGION 1..65: note = Synthetic peptide
GAPGGGGSGGGGSGGGGSAAAASGGGPSGGGGSGGGGSGGGGSGGGGSGGGGSGGGGSAAAAGAP

SEQ ID NO: 190 - protein, 70 aa, synthetic construct
  REGION 1..70: note = Synthetic peptide
GAPGGGGSGGGGSGGGGSAAAASGGGPSGGGGSGGGGSGGGGSGGGGSGGGGSGGGGSGGGGSAAAAGAP

SEQ ID NO: 191 - protein, 45 aa, synthetic construct
  REGION 1..45: note = Synthetic peptide
GAPGGGGSGGGGSGGGGSGGGGSAAAASGGGPSGGGGSAAAAGAP

SEQ ID NO: 192 - protein, 50 aa, synthetic construct
  REGION 1..50: note = Synthetic peptide
GAPGGGGSGGGGSGGGGSGGGGSAAAASGGGPSGGGGSGGGGSAAAAGAP

SEQ ID NO: 193 - protein, 55 aa, synthetic construct
  REGION 1..55: note = Synthetic peptide
GAPGGGGSGGGGSGGGGSGGGGSAAAASGGGPSGGGGSGGGGSGGGGSAAAAGAP

SEQ ID NO: 194 - protein, 60 aa, synthetic construct
  REGION 1..60: note = Synthetic peptide
GAPGGGGSGGGGSGGGGSGGGGSAAAASGGGPSGGGGSGGGGSGGGGSGGGGSAAAAGAP

SEQ ID NO: 195 - protein, 65 aa, synthetic construct
  REGION 1..65: note = Synthetic peptide
GAPGGGGSGGGGSGGGGSGGGGSAAAASGGGPSGGGGSGGGGSGGGGSGGGGSGGGGSAAAAGAP

SEQ ID NO: 196 - protein, 70 aa, synthetic construct
  REGION 1..70: note = Synthetic peptide
GAPGGGGSGGGGSGGGGSGGGGSAAAASGGGPSGGGGSGGGGSGGGGSGGGGSGGGGSGGGGSAAAAGAP

SEQ ID NO: 197 - protein, 75 aa, synthetic construct
  REGION 1..75: note = Synthetic peptide
GAPGGGGSGGGGSGGGGSGGGGSAAAASGGGPSGGGGSGGGGSGGGGSGGGGSGGGGSGGGGSGGGGSAAAAGAP

SEQ ID NO: 198 - protein, 50 aa, synthetic construct
  REGION 1..50: note = Synthetic peptide
GAPGGGGSGGGGSGGGGSGGGGSGGGGSAAAASGGGPSGGGGSAAAAGAP

SEQ ID NO: 199 - protein, 55 aa, synthetic construct
  REGION 1..55: note = Synthetic peptide
GAPGGGGSGGGGSGGGGSGGGGSGGGGSAAAASGGGPSGGGGSGGGGSAAAAGAP

SEQ ID NO: 200 - protein, 60 aa, synthetic construct
  REGION 1..60: note = Synthetic peptide
GAPGGGGSGGGGSGGGGSGGGGSGGGGSAAAASGGGPSGGGGSGGGGSGGGGSAAAAGAP

SEQ ID NO: 201 - protein, 65 aa, synthetic construct
  REGION 1..65: note = Synthetic peptide
GAPGGGGSGGGGSGGGGSGGGGSGGGGSAAAASGGGPSGGGGSGGGGSGGGGSGGGGSAAAAGAP

SEQ ID NO: 202 - protein, 70 aa, synthetic construct
  REGION 1..70: note = Synthetic peptide
GAPGGGGSGGGGSGGGGSGGGGSGGGGSAAAASGGGPSGGGGSGGGGSGGGGSGGGGSGGGGSAAAAGAP

SEQ ID NO: 203 - protein, 75 aa, synthetic construct
  REGION 1..75: note = Synthetic peptide
GAPGGGGSGGGGSGGGGSGGGGSGGGGSAAAASGGGPSGGGGSGGGGSGGGGSGGGGSGGGGSGGGGSAAAAGAP

SEQ ID NO: 204 - protein, 80 aa, synthetic construct
  REGION 1..80: note = Synthetic peptide
GAPGGGGSGGGGSGGGGSGGGGSGGGGSAAAASGGGPSGGGGSGGGGSGGGGSGGGGSGGGGSGGGGSGGGGSAAAAGAP

SEQ ID NO: 205 - protein, 55 aa, synthetic construct
  REGION 1..55: note = Synthetic peptide
GAPGGGGSGGGGSGGGGSGGGGSGGGGSGGGGSAAAASGGGPSGGGGSAAAAGAP

SEQ ID NO: 206 - protein, 60 aa, synthetic construct
  REGION 1..60: note = Synthetic peptide
GAPGGGGSGGGGSGGGGSGGGGSGGGGSGGGGSAAAASGGGPSGGGGSGGGGSAAAAGAP

SEQ ID NO: 207 - protein, 65 aa, synthetic construct
  REGION 1..65: note = Synthetic peptide
GAPGGGGSGGGGSGGGGSGGGGSGGGGSGGGGSAAAASGGGPSGGGGSGGGGSGGGGSAAAAGAP

SEQ ID NO: 208 - protein, 70 aa, synthetic construct
  REGION 1..70: note = Synthetic peptide
GAPGGGGSGGGGSGGGGSGGGGSGGGGSGGGGSAAAASGGGPSGGGGSGGGGSGGGGSGGGGSAAAAGAP

SEQ ID NO: 209 - protein, 75 aa, synthetic construct
  REGION 1..75: note = Synthetic peptide
GAPGGGGSGGGGSGGGGSGGGGSGGGGSGGGGSAAAASGGGPSGGGGSGGGGSGGGGSGGGGSGGGGSAAAAGAP

SEQ ID NO: 210 - protein, 80 aa, synthetic construct
  REGION 1..80: note = Synthetic peptide
GAPGGGGSGGGGSGGGGSGGGGSGGGGSGGGGSAAAASGGGPSGGGGSGGGGSGGGGSGGGGSGGGGSGGGGSAAAAGAP

SEQ ID NO: 211 - protein, 85 aa, synthetic construct
  REGION 1..85: note = Synthetic peptide
GAPGGGGSGGGGSGGGGSGGGGSGGGGSGGGGSAAAASGGGPSGGGGSGGGGSGGGGSGGGGSGGGGSGGGGSGGGGSAAAAGAP

SEQ ID NO: 212 - protein, 60 aa, synthetic construct
  REGION 1..60: note = Synthetic peptide
GAPGGGGSGGGGSGGGGSGGGGSGGGGSGGGGSGGGGSAAAASGGGPSGGGGSAAAAGAP

SEQ ID NO: 213 - protein, 65 aa, synthetic construct
  REGION 1..65: note = Synthetic peptide
GAPGGGGSGGGGSGGGGSGGGGSGGGGSGGGGSGGGGSAAAASGGGPSGGGGSGGGGSAAAAGAP

SEQ ID NO: 214 - protein, 70 aa, synthetic construct
  REGION 1..70: note = Synthetic peptide
GAPGGGGSGGGGSGGGGSGGGGSGGGGSGGGGSGGGGSAAAASGGGPSGGGGSGGGGSGGGGSAAAAGAP

SEQ ID NO: 215 - protein, 75 aa, synthetic construct
  REGION 1..75: note = Synthetic peptide
GAPGGGGSGGGGSGGGGSGGGGSGGGGSGGGGSGGGGSAAAASGGGPSGGGGSGGGGSGGGGSGGGGSAAAAGAP

SEQ ID NO: 216 - protein, 80 aa, synthetic construct
  REGION 1..80: note = Synthetic peptide
GAPGGGGSGGGGSGGGGSGGGGSGGGGSGGGGSGGGGSAAAASGGGPSGGGGSGGGGSGGGGSGGGGSGGGGSAAAAGAP

SEQ ID NO: 217 - protein, 85 aa, synthetic construct
  REGION 1..85: note = Synthetic peptide
GAPGGGGSGGGGSGGGGSGGGGSGGGGSGGGGSGGGGSAAAASGGGPSGGGGSGGGGSGGGGSGGGGSGGGGSGGGGSAAAAGAP

SEQ ID NO: 218 - protein, 90 aa, synthetic construct
  REGION 1..90: note = Synthetic peptide
GAPGGGGSGGGGSGGGGSGGGGSGGGGSGGGGSGGGGSAAAASGGGPSGGGGSGGGGSGGGGSGGGGSGGGGSGGGGSGGGGSAAAAGAP

SEQ ID NO: 219 - protein, 17 aa, synthetic construct
  REGION 1..17: note = Synthetic peptide
GAPGGGGSPAPAPXGAP

SEQ ID NO: 220 - protein, 18 aa, synthetic construct
  REGION 1..18: note = Synthetic peptide
GAPGGGGSPAPAPXXGAP

SEQ ID NO: 221 - protein, 19 aa, synthetic construct
GAPGGGGSPAPAPXXXGAP

SEQ ID NO: 222 - protein, 20 aa, synthetic construct
  REGION 1..20: note = Synthetic peptide
GAPGGGGSPAPAPXXXXGAP

SEQ ID NO: 223 - protein, 21 aa, synthetic construct
  REGION 1..21: note = Synthetic peptide
GAPGGGGSPAPAPXXXXXGAP

SEQ ID NO: 224 - protein, 22 aa, synthetic construct
  REGION 1..22: note = Synthetic peptide
GAPGGGGSPAPAPXXXXXXGAP

SEQ ID NO: 225 - protein, 23 aa, synthetic construct
  REGION 1..23: note = Synthetic peptide
GAPGGGGSPAPAPXXXXXXXGAP

SEQ ID NO: 226 - protein, 22 aa, synthetic construct
  REGION 1..22: note = Synthetic peptide
GAPGGGGSGGGGSPAPAPXGAP

SEQ ID NO: 227 - protein, 23 aa, synthetic construct
  REGION 1..23: note = Synthetic peptide
GAPGGGGSGGGGSPAPAPXXGAP

SEQ ID NO: 228 - protein, 24 aa, synthetic construct
  REGION 1..24: note = Synthetic peptide
GAPGGGGSGGGGSPAPAPXXXGAP

SEQ ID NO: 229 - protein, 25 aa, synthetic construct
  REGION 1..25: note = Synthetic peptide
GAPGGGGSGGGGSPAPAPXXXXGAP

SEQ ID NO: 230 - protein, 26 aa, synthetic construct
  REGION 1..26: note = Synthetic peptide
GAPGGGGSGGGGSPAPAPXXXXXGAP

SEQ ID NO: 231 - protein, 27 aa, synthetic construct
  REGION 1..27: note = Synthetic peptide
GAPGGGGSGGGGSPAPAPXXXXXXGAP

SEQ ID NO: 232 - protein, 28 aa, synthetic construct
  REGION 1..28: note = Synthetic peptide
GAPGGGGSGGGGSPAPAPXXXXXXXGAP

SEQ ID NO: 233 - protein, 27 aa, synthetic construct
  REGION 1..27: note = Synthetic peptide
GAPGGGGSGGGGSGGGGSPAPAPXGAP

SEQ ID NO: 234 - protein, 28 aa, synthetic construct
  REGION 1..28: note = Synthetic peptide
GAPGGGGSGGGGSGGGGSPAPAPXXGAP

SEQ ID NO: 235 - protein, 29 aa, synthetic construct
  REGION 1..29: note = Synthetic peptide
GAPGGGGSGGGGSGGGGSPAPAPXXXGAP

SEQ ID NO: 236 - protein, 30 aa, synthetic construct
  REGION 1..30: note = Synthetic peptide
GAPGGGGSGGGGSGGGGSPAPAPXXXXGAP

SEQ ID NO: 237 - protein, 31 aa, synthetic construct
  REGION 1..31: note = Synthetic peptide
GAPGGGGSGGGGSGGGGSPAPAPXXXXXGAP

SEQ ID NO: 238 - protein, 32 aa, synthetic construct
  REGION 1..32: note = Synthetic peptide
GAPGGGGSGGGGSGGGGSPAPAPXXXXXXGAP

SEQ ID NO: 239 - protein, 33 aa, synthetic construct
  REGION 1..33: note = Synthetic peptide
GAPGGGGSGGGGSGGGGSPAPAPXXXXXXXGAP

SEQ ID NO: 240 - protein, 32 aa, synthetic construct
  REGION 1..32: note = Synthetic peptide
GAPGGGGSGGGGSGGGGSGGGGSPAPAPXGAP

SEQ ID NO: 241 - protein, 33 aa, synthetic construct
  REGION 1..33: note = Synthetic peptide
GAPGGGGSGGGGSGGGGSGGGGSPAPAPXXGAP

SEQ ID NO: 242 - protein, 34 aa, synthetic construct
  REGION 1..34: note = Synthetic peptide
GAPGGGGSGGGGSGGGGSGGGGSPAPAPXXXGAP

SEQ ID NO: 243 - protein, 35 aa, synthetic construct
  REGION 1..35: note = Synthetic peptide
GAPGGGGSGGGGSGGGGSGGGGSPAPAPXXXXGAP

SEQ ID NO: 244 - protein, 36 aa, synthetic construct
  REGION 1..36: note = Synthetic peptide
GAPGGGGSGGGGSGGGGSGGGGSPAPAPXXXXXGAP

SEQ ID NO: 245 - protein, 37 aa, synthetic construct
  REGION 1..37: note = Synthetic peptide
GAPGGGGSGGGGSGGGGSGGGGSPAPAPXXXXXXGAP

SEQ ID NO: 246 - protein, 38 aa, synthetic construct
  REGION 1..38: note = Synthetic peptide
GAPGGGGSGGGGSGGGGSGGGGSPAPAPXXXXXXXGAP

SEQ ID NO: 247 - protein, 37 aa, synthetic construct
  REGION 1..37: note = Synthetic peptide
GAPGGGGSGGGGSGGGGSGGGGSGGGGSPAPAPXGAP

SEQ ID NO: 248 - protein, 38 aa, synthetic construct
  REGION 1..38: note = Synthetic peptide
GAPGGGGSGGGGSGGGGSGGGGSGGGGSPAPAPXXGAP

SEQ ID NO: 249 - protein, 39 aa, synthetic construct
  REGION 1..39: note = Synthetic peptide
GAPGGGGSGGGGSGGGGSGGGGSGGGGSPAPAPXXXGAP

SEQ ID NO: 250 - protein, 40 aa, synthetic construct
  REGION 1..40: note = Synthetic peptide
GAPGGGGSGGGGSGGGGSGGGGSGGGGSPAPAPXXXXGAP

SEQ ID NO: 251 - protein, 41 aa, synthetic construct
  REGION 1..41: note = Synthetic peptide
GAPGGGGSGGGGSGGGGSGGGGSGGGGSPAPAPXXXXXGAP

SEQ ID NO: 252 - protein, 42 aa, synthetic construct
  REGION 1..42: note = Synthetic peptide
GAPGGGGSGGGGSGGGGSGGGGSGGGGSPAPAPXXXXXXGAP

SEQ ID NO: 253 - protein, 43 aa, synthetic construct
  REGION 1..43: note = Synthetic peptide
GAPGGGGSGGGGSGGGGSGGGGSGGGGSPAPAPXXXXXXXGAP

SEQ ID NO: 254 - protein, 42 aa, synthetic construct
  REGION 1..42: note = Synthetic peptide
GAPGGGGSGGGGSGGGGSGGGGSGGGGSGGGGSPAPAPXGAP

SEQ ID NO: 255 - protein, 43 aa, synthetic construct
  REGION 1..43: note = Synthetic peptide
GAPGGGGSGGGGSGGGGSGGGGSGGGGSGGGGSPAPAPXXGAP

SEQ ID NO: 256 - protein, 44 aa, synthetic construct
  REGION 1..44: note = Synthetic peptide
GAPGGGGSGGGGSGGGGSGGGGSGGGGSGGGGSPAPAPXXXGAP

SEQ ID NO: 257 - protein, 45 aa, synthetic construct
  REGION 1..45: note = Synthetic peptide
GAPGGGGSGGGGSGGGGSGGGGSGGGGSGGGGSPAPAPXXXXGAP

SEQ ID NO: 258 - protein, 46 aa, synthetic construct
  REGION 1..46: note = Synthetic peptide
GAPGGGGSGGGGSGGGGSGGGGSGGGGSGGGGSPAPAPXXXXXGAP

SEQ ID NO: 259 - protein, 47 aa, synthetic construct
  REGION 1..47: note = Synthetic peptide
GAPGGGGSGGGGSGGGGSGGGGSGGGGSGGGGSPAPAPXXXXXXGAP

SEQ ID NO: 260 - protein, 48 aa, synthetic construct
  REGION 1..48: note = Synthetic peptide
GAPGGGGSGGGGSGGGGSGGGGSGGGGSGGGGSPAPAPXXXXXXXGAP

SEQ ID NO: 261 - protein, 47 aa, synthetic construct
  REGION 1..47: note = Synthetic peptide
GAPGGGGSGGGGSGGGGSGGGGSGGGGSGGGGSGGGGSPAPAPXGAP

SEQ ID NO: 262 - protein, 48 aa, synthetic construct
  REGION 1..48: note = Synthetic peptide
GAPGGGGSGGGGSGGGGSGGGGSGGGGSGGGGSGGGGSPAPAPXXGAP

SEQ ID NO: 263 - protein, 49 aa, synthetic construct
  REGION 1..49: note = Synthetic peptide
GAPGGGGSGGGGSGGGGSGGGGSGGGGSGGGGSGGGGSPAPAPXXXGAP

SEQ ID NO: 264 - protein, 50 aa, synthetic construct
  REGION 1..50: note = Synthetic peptide
GAPGGGGSGGGGSGGGGSGGGGSGGGGSGGGGSGGGGSPAPAPXXXXGAP

SEQ ID NO: 265 - protein, 51 aa, synthetic construct
  REGION 1..51: note = Synthetic peptide
GAPGGGGSGGGGSGGGGSGGGGSGGGGSGGGGSGGGGSPAPAPXXXXXGAP

SEQ ID NO: 266 - protein, 52 aa, synthetic construct
  REGION 1..52: note = Synthetic peptide
GAPGGGGSGGGGSGGGGSGGGGSGGGGSGGGGSGGGGSPAPAPXXXXXXGAP

SEQ ID NO: 267 - protein, 53 aa, synthetic construct
  REGION 1..53: note = Synthetic peptide
GAPGGGGSGGGGSGGGGSGGGGSGGGGSGGGGSGGGGSPAPAPXXXXXXXGAP

SEQ ID NO: 268 - protein, 18 aa, synthetic construct
  REGION 1..18: note = Synthetic peptide
GAPGGGGSPAPAPTXGAP

SEQ ID NO: 269 - protein, 19 aa, synthetic construct
  REGION 1..19: note = Synthetic peptide
GAPGGGGSPAPAPTXXGAP

SEQ ID NO: 270 - protein, 20 aa, synthetic construct
  REGION 1..20: note = Synthetic peptide
GAPGGGGSPAPAPTXXXGAP

SEQ ID NO: 271 - protein, 21 aa, synthetic construct
  REGION 1..21: note = Synthetic peptide
GAPGGGGSPAPAPTXXXXGAP

SEQ ID NO: 272 - protein, 22 aa, synthetic construct
  REGION 1..22: note = Synthetic peptide
GAPGGGGSPAPAPTXXXXXGAP

SEQ ID NO: 273 - protein, 23 aa, synthetic construct
  REGION 1..23: note = Synthetic peptide
GAPGGGGSPAPAPTXXXXXXGAP

SEQ ID NO: 274 - protein, 24 aa, synthetic construct
  REGION 1..24: note = Synthetic peptide
GAPGGGGSPAPAPTXXXXXXXGAP

SEQ ID NO: 275 - protein, 23 aa, synthetic construct
  REGION 1..23: note = Synthetic peptide
GAPGGGGSGGGGSPAPAPTXGAP

SEQ ID NO: 276 - protein, 24 aa, synthetic construct
  REGION 1..24: note = Synthetic peptide
GAPGGGGSGGGGSPAPAPTXXGAP

SEQ ID NO: 277 - protein, 25 aa, synthetic construct
  REGION 1..25: note = Synthetic peptide
GAPGGGGSGGGGSPAPAPTXXXGAP

SEQ ID NO: 278 - protein, 26 aa, synthetic construct
  REGION 1..26: note = Synthetic peptide
GAPGGGGSGGGGSPAPAPTXXXXGAP

SEQ ID NO: 279 - protein, 27 aa, synthetic construct
  REGION 1..27: note = Synthetic peptide
GAPGGGGSGGGGSPAPAPTXXXXXGAP

SEQ ID NO: 280 - protein, 28 aa, synthetic construct
  REGION 1..28: note = Synthetic peptide
GAPGGGGSGGGGSPAPAPTXXXXXXGAP

SEQ ID NO: 281 - protein, 29 aa, synthetic construct
  REGION 1..29: note = Synthetic peptide
GAPGGGGSGGGGSPAPAPTXXXXXXXGAP

SEQ ID NO: 282 - protein, 28 aa, synthetic construct
  REGION 1..28: note = Synthetic peptide
GAPGGGGSGGGGSGGGGSPAPAPTXGAP

SEQ ID NO: 283 - protein, 29 aa, synthetic construct
  REGION 1..29: note = Synthetic peptide
GAPGGGGSGGGGSGGGGSPAPAPTXXGAP

SEQ ID NO: 284 - protein, 30 aa, synthetic construct
  REGION 1..30: note = Synthetic peptide
GAPGGGGSGGGGSGGGGSPAPAPTXXXGAP

SEQ ID NO: 285 - protein, 31 aa, synthetic construct
  REGION 1..31: note = Synthetic peptide
GAPGGGGSGGGGSGGGGSPAPAPTXXXXGAP

SEQ ID NO: 286 - protein, 32 aa, synthetic construct
  REGION 1..32: note = Synthetic peptide
GAPGGGGSGGGGSGGGGSPAPAPTXXXXXGAP

SEQ ID NO: 287 - protein, 33 aa, synthetic construct
  REGION 1..33: note = Synthetic peptide
GAPGGGGSGGGGSGGGGSPAPAPTXXXXXXGAP

SEQ ID NO: 288 - protein, 34 aa, synthetic construct
  REGION 1..34: note = Synthetic peptide
GAPGGGGSGGGGSGGGGSPAPAPTXXXXXXXGAP

SEQ ID NO: 289 - protein, 33 aa, synthetic construct
  REGION 1..33: note = Synthetic peptide
GAPGGGGSGGGGSGGGGSGGGGSPAPAPTXGAP

SEQ ID NO: 290 - protein, 34 aa, synthetic construct
  REGION 1..34: note = Synthetic peptide
GAPGGGGSGGGGSGGGGSGGGGSPAPAPTXXGAP

SEQ ID NO: 291 - protein, 35 aa, synthetic construct
  REGION 1..35: note = Synthetic peptide
GAPGGGGSGGGGSGGGGSGGGGSPAPAPTXXXGAP

SEQ ID NO: 292 - protein, 36 aa, synthetic construct
  REGION 1..36: note = Synthetic peptide
GAPGGGGSGGGGSGGGGSGGGGSPAPAPTXXXXGAP

SEQ ID NO: 293 - protein, 37 aa, synthetic construct
  REGION 1..37: note = Synthetic peptide
GAPGGGGSGGGGSGGGGSGGGGSPAPAPTXXXXXGAP

SEQ ID NO: 294 - protein, 38 aa, synthetic construct
  REGION 1..38: note = Synthetic peptide
GAPGGGGSGGGGSGGGGSGGGGSPAPAPTXXXXXXGAP

SEQ ID NO: 295 - protein, 39 aa, synthetic construct
  REGION 1..39: note = Synthetic peptide
GAPGGGGSGGGGSGGGGSGGGGSPAPAPTXXXXXXXGAP

SEQ ID NO: 296 - protein, 38 aa, synthetic construct
  REGION 1..38: note = Synthetic peptide
GAPGGGGSGGGGSGGGGSGGGGSGGGGSPAPAPTXGAP

SEQ ID NO: 297 - protein, 39 aa, synthetic construct
  REGION 1..39: note = Synthetic peptide
GAPGGGGSGGGGSGGGGSGGGGSGGGGSPAPAPTXXGAP

SEQ ID NO: 298 - protein, 40 aa, synthetic construct
  REGION 1..40: note = Synthetic peptide
GAPGGGGSGGGGSGGGGSGGGGSGGGGSPAPAPTXXXGAP

SEQ ID NO: 299 - protein, 41 aa, synthetic construct
  REGION 1..41: note = Synthetic peptide
GAPGGGGSGGGGSGGGGSGGGGSGGGGSPAPAPTXXXXGAP

SEQ ID NO: 300 - protein, 42 aa, synthetic construct
  REGION 1..42: note = Synthetic peptide
GAPGGGGSGGGGSGGGGSGGGGSGGGGSPAPAPTXXXXXGAP

SEQ ID NO: 301 - protein, 43 aa, synthetic construct
  REGION 1..43: note = Synthetic peptide
GAPGGGGSGGGGSGGGGSGGGGSGGGGSPAPAPTXXXXXXGAP

SEQ ID NO: 302 - protein, 44 aa, synthetic construct
  REGION 1..44: note = Synthetic peptide
GAPGGGGSGGGGSGGGGSGGGGSGGGGSPAPAPTXXXXXXXGAP

SEQ ID NO: 303 - protein, 43 aa, synthetic construct
  REGION 1..43: note = Synthetic peptide
GAPGGGGSGGGGSGGGGSGGGGSGGGGSGGGGSPAPAPTXGAP

SEQ ID NO: 304 - protein, 44 aa, synthetic construct
  REGION 1..44: note = Synthetic peptide
GAPGGGGSGGGGSGGGGSGGGGSGGGGSGGGGSPAPAPTXXGAP

SEQ ID NO: 305 - protein, 45 aa, synthetic construct
  REGION 1..45: note = Synthetic peptide
GAPGGGGSGGGGSGGGGSGGGGSGGGGSGGGGSPAPAPTXXXGAP

SEQ ID NO: 306 - protein, 46 aa, synthetic construct
  REGION 1..46: note = Synthetic peptide
GAPGGGGSGGGGSGGGGSGGGGSGGGGSGGGGSPAPAPTXXXXGAP

SEQ ID NO: 307 - protein, 47 aa, synthetic construct
  REGION 1..47: note = Synthetic peptide
GAPGGGGSGGGGSGGGGSGGGGSGGGGSGGGGSPAPAPTXXXXXGAP

SEQ ID NO: 308 - protein, 48 aa, synthetic construct
  REGION 1..48: note = Synthetic peptide
GAPGGGGSGGGGSGGGGSGGGGSGGGGSGGGGSPAPAPTXXXXXXGAP

SEQ ID NO: 309 - protein, 49 aa, synthetic construct
  REGION 1..49: note = Synthetic peptide
GAPGGGGSGGGGSGGGGSGGGGSGGGGSGGGGSPAPAPTXXXXXXXGAP

SEQ ID NO: 310 - protein, 48 aa, synthetic construct
  REGION 1..48: note = Synthetic peptide
GAPGGGGSGGGGSGGGGSGGGGSGGGGSGGGGSGGGGSPAPAPTXGAP

SEQ ID NO: 311 - protein, 49 aa, synthetic construct
  REGION 1..49: note = Synthetic peptide
GAPGGGGSGGGGSGGGGSGGGGSGGGGSGGGGSGGGGSPAPAPTXXGAP

SEQ ID NO: 312 - protein, 50 aa, synthetic construct
  REGION 1..50: note = Synthetic peptide
GAPGGGGSGGGGSGGGGSGGGGSGGGGSGGGGSGGGGSPAPAPTXXXGAP

SEQ ID NO: 313 - protein, 51 aa, synthetic construct
  REGION 1..51: note = Synthetic peptide
GAPGGGGSGGGGSGGGGSGGGGSGGGGSGGGGSGGGGSPAPAPTXXXXGAP

SEQ ID NO: 314 - protein, 52 aa, synthetic construct
  REGION 1..52: note = Synthetic peptide
GAPGGGGSGGGGSGGGGSGGGGSGGGGSGGGGSGGGGSPAPAPTXXXXXGAP

SEQ ID NO: 315 - protein, 53 aa, synthetic construct
  REGION 1..53: note = Synthetic peptide
GAPGGGGSGGGGSGGGGSGGGGSGGGGSGGGGSGGGGSPAPAPTXXXXXXGAP

SEQ ID NO: 316 - protein, 54 aa, synthetic construct
  REGION 1..54: note = Synthetic peptide
GAPGGGGSGGGGSGGGGSGGGGSGGGGSGGGGSGGGGSPAPAPTXXXXXXXGAP

SEQ ID NO: 317 - protein, 10 aa, synthetic construct
  REGION 1..10: note = Synthetic peptide
GGGGAGGGGA

SEQ ID NO: 318 - protein, 25 aa, synthetic construct
  REGION 1..25: note = Synthetic peptide
GGGGAGGGGAGGGGAGGGGAGGGGA

SEQ ID NO: 319 - protein, 30 aa, synthetic construct
  REGION 1..30: note = Synthetic peptide
GGGGAGGGGAGGGGAGGGGAGGGGAGGGGA

SEQ ID NO: 320 - protein, 35 aa, synthetic construct
  REGION 1..35: note = Synthetic peptide
GGGGAGGGGAGGGGAGGGGAGGGGAGGGGAGGGGA

SEQ ID NO: 321 - protein, 10 aa, synthetic construct
  REGION 1..10: note = Synthetic peptide
GGGGAGGGPS

SEQ ID NO: 322 - protein, 15 aa, synthetic construct
  REGION 1..15: note = Synthetic peptide
GGGGAGGGGAGGGPS

SEQ ID NO: 323 - protein, 20 aa, synthetic construct
  REGION 1..20: note = Synthetic peptide
GGGGAGGGGAGGGGAGGGPS

SEQ ID NO: 324 - protein, 30 aa, synthetic construct
  REGION 1..30: note = Synthetic peptide
GGGGAGGGGAGGGGAGGGGAGGGGAGGGPS

SEQ ID NO: 325 - protein, 35 aa, synthetic construct
  REGION 1..35: note = Synthetic peptide
GGGGAGGGGAGGGGAGGGGAGGGGAGGGGAGGGPS

SEQ ID NO: 326 - protein, 40 aa, synthetic construct
  REGION 1..40: note = Synthetic peptide
GGGGAGGGGAGGGGAGGGGAGGGGAGGGGAGGGGAGGGPS

SEQ ID NO: 327 - protein, 13 aa, synthetic construct
  REGION 1..13: note = Synthetic peptide
GAPGGGGAGGGPS

SEQ ID NO: 328 - protein, 18 aa, synthetic construct
  REGION 1..18: note = Synthetic peptide
GAPGGGGAGGGGAGGGPS

SEQ ID NO: 329 - protein, 23 aa, synthetic construct
  REGION 1..23: note = Synthetic peptide
GAPGGGGAGGGGAGGGGAGGGPS

SEQ ID NO: 330 - protein, 28 aa, synthetic construct
  REGION 1..28: note = Synthetic peptide
GAPGGGGAGGGGAGGGGAGGGGAGGGPS

SEQ ID NO: 331 - protein, 33 aa, synthetic construct
  REGION 1..33: note = Synthetic peptide
GAPGGGGAGGGGAGGGGAGGGGAGGGGAGGGPS

SEQ ID NO: 332 - protein, 38 aa, synthetic construct
  REGION 1..38: note = Synthetic peptide
GAPGGGGAGGGGAGGGGAGGGGAGGGGAGGGGAGGGPS

SEQ ID NO: 333 - protein, 43 aa, synthetic construct
  REGION 1..43: note = Synthetic peptide
GAPGGGGAGGGGAGGGGAGGGGAGGGGAGGGGAGGGGAGGGPS

SEQ ID NO: 334 - protein, 16 aa, synthetic construct
  REGION 1..16: note = Synthetic peptide
GAPGGGGAGGGPSGAP

SEQ ID NO: 335 - protein, 21 aa, synthetic construct
  REGION 1..21: note = Synthetic peptide
GAPGGGGAGGGGAGGGPSGAP

SEQ ID NO: 336 - protein, 26 aa, synthetic construct
  REGION 1..26: note = Synthetic peptide
GAPGGGGAGGGGAGGGGAGGGPSGAP

SEQ ID NO: 337 - protein, 31 aa, synthetic construct; identical to SEQ ID NO: 72
  REGION 1..31: note = Synthetic peptide

SEQ ID NO: 338 - protein, 36 aa, synthetic construct
  REGION 1..36: note = Synthetic peptide
GAPGGGGAGGGGAGGGGAGGGGAGGGGAGGGPSGAP

SEQ ID NO: 339 - protein, 41 aa, synthetic construct
  REGION 1..41: note = Synthetic peptide
GAPGGGGAGGGGAGGGGAGGGGAGGGGAGGGGAGGGPSGAP

SEQ ID NO: 340 - protein, 46 aa, synthetic construct
  REGION 1..46: note = Synthetic peptide
GAPGGGGAGGGGAGGGGAGGGGAGGGGAGGGGAGGGGAGGGPSGAP

SEQ ID NO: 341 - protein, 21 aa, synthetic construct
  REGION 1..21: note = Synthetic peptide
GAPGGGGAGGGPSGGGGAGAP

SEQ ID NO: 342 - protein, 26 aa, synthetic construct
  REGION 1..26: note = Synthetic peptide
GAPGGGGAGGGPSGGGGAGGGGAGAP

SEQ ID NO: 343 - protein, 31 aa, synthetic construct
  REGION 1..31: note = Synthetic peptide
GAPGGGGAGGGPSGGGGAGGGGAGGGGAGAP

SEQ ID NO: 344 - protein, 36 aa, synthetic construct
  REGION 1..36: note = Synthetic peptide
GAPGGGGAGGGPSGGGGAGGGGAGGGGAGGGGAGAP

SEQ ID NO: 345 - protein, 41 aa, synthetic construct
  REGION 1..41: note = Synthetic peptide
GAPGGGGAGGGPSGGGGAGGGGAGGGGAGGGGAGGGGAGAP

SEQ ID NO: 346 - protein, 46 aa, synthetic construct
  REGION 1..46: note = Synthetic peptide
GAPGGGGAGGGPSGGGGAGGGGAGGGGAGGGGAGGGGAGGGGAGAP

SEQ ID NO: 347 - protein, 51 aa, synthetic construct
  REGION 1..51: note = Synthetic peptide
GAPGGGGAGGGPSGGGGAGGGGAGGGGAGGGGAGGGGAGGGGAGGGGAGAP

SEQ ID NO: 348 - protein, 26 aa, synthetic construct
  REGION 1..26: note = Synthetic peptide
GAPGGGGAGGGGAGGGPSGGGGAGAP

SEQ ID NO: 349 - protein, 31 aa, synthetic construct
  REGION 1..31: note = Synthetic peptide
GAPGGGGAGGGGAGGGPSGGGGAGGGGAGAP

SEQ ID NO: 350 - protein, 36 aa, synthetic construct
  REGION 1..36: note = Synthetic peptide
GAPGGGGAGGGGAGGGPSGGGGAGGGGAGGGGAGAP

SEQ ID NO: 351 - protein, 41 aa, synthetic construct
  REGION 1..41: note = Synthetic peptide
GAPGGGGAGGGGAGGGPSGGGGAGGGGAGGGGAGGGGAGAP

SEQ ID NO: 352 - protein, 46 aa, synthetic construct
  REGION 1..46: note = Synthetic peptide
GAPGGGGAGGGGAGGGPSGGGGAGGGGAGGGGAGGGGAGGGGAGAP

SEQ ID NO: 353 - protein, 51 aa, synthetic construct
  REGION 1..51: note = Synthetic peptide
GAPGGGGAGGGGAGGGPSGGGGAGGGGAGGGGAGGGGAGGGGAGGGGAGAP

SEQ ID NO: 354 - protein, 56 aa, synthetic construct
  REGION 1..56: note = Synthetic peptide
GAPGGGGAGGGGAGGGPSGGGGAGGGGAGGGGAGGGGAGGGGAGGGGAGGGGAGAP

SEQ ID NO: 355 - protein, 31 aa, synthetic construct
  REGION 1..31: note = Synthetic peptide
GAPGGGGAGGGGAGGGGAGGGPSGGGGAGAP

SEQ ID NO: 356 - protein, 36 aa, synthetic construct
  REGION 1..36: note = Synthetic peptide
GAPGGGGAGGGGAGGGGAGGGPSGGGGAGGGGAGAP

SEQ ID NO: 357 - protein, 41 aa, synthetic construct
  REGION 1..41: note = Synthetic peptide
GAPGGGGAGGGGAGGGGAGGGPSGGGGAGGGGAGGGGAGAP

SEQ ID NO: 358 - protein, 46 aa, synthetic construct
  REGION 1..46: note = Synthetic peptide
GAPGGGGAGGGGAGGGGAGGGPSGGGGAGGGGAGGGGAGGGGAGAP

SEQ ID NO: 359 - protein, 51 aa, synthetic construct
  REGION 1..51: note = Synthetic peptide
GAPGGGGAGGGGAGGGGAGGGPSGGGGAGGGGAGGGGAGGGGAGGGGAGAP

SEQ ID NO: 360 - protein, 56 aa, synthetic construct
  REGION 1..56: note = Synthetic peptide
GAPGGGGAGGGGAGGGGAGGGPSGGGGAGGGGAGGGGAGGGGAGGGGAGGGGAGAP

SEQ ID NO: 361 - protein, 61 aa, synthetic construct
  REGION 1..61: note = Synthetic peptide
GAPGGGGAGGGGAGGGGAGGGPSGGGGAGGGGAGGGGAGGGGAGGGGAGGGGAGGGGAGAP

SEQ ID NO: 362 - protein, 36 aa, synthetic construct
  REGION 1..36: note = Synthetic peptide
GAPGGGGAGGGGAGGGGAGGGGAGGGPSGGGGAGAP

SEQ ID NO: 363 - protein, 41 aa, synthetic construct
  REGION 1..41: note = Synthetic peptide
GAPGGGGAGGGGAGGGGAGGGGAGGGPSGGGGAGGGGAGAP

SEQ ID NO: 364 - protein, 46 aa, synthetic construct
  REGION 1..46: note = Synthetic peptide
GAPGGGGAGGGGAGGGGAGGGGAGGGPSGGGGAGGGGAGGGGAGAP

SEQ ID NO: 365 - protein, 51 aa, synthetic construct
  REGION 1..51: note = Synthetic peptide
GAPGGGGAGGGGAGGGGAGGGGAGGGPSGGGGAGGGGAGGGGAGGGGAGAP

SEQ ID NO: 366 - protein, 56 aa, synthetic construct
  REGION 1..56: note = Synthetic peptide
GAPGGGGAGGGGAGGGGAGGGGAGGGPSGGGGAGGGGAGGGGAGGGGAGGGGAGAP

SEQ ID NO: 367 - protein, 61 aa, synthetic construct
  REGION 1..61: note = Synthetic peptide
GAPGGGGAGGGGAGGGGAGGGGAGGGPSGGGGAGGGGAGGGGAGGGGAGGGGAGGGGAGAP

SEQ ID NO: 368 - protein, 66 aa, synthetic construct
  REGION 1..66: note = Synthetic peptide
GAPGGGGAGGGGAGGGGAGGGGAGGGPSGGGGAGGGGAGGGGAGGGGAGGGGAGGGGAGGGGAGAP

SEQ ID NO: 369 - protein, 41 aa, synthetic construct
  REGION 1..41: note = Synthetic peptide
GAPGGGGAGGGGAGGGGAGGGGAGGGGAGGGPSGGGGAGAP

SEQ ID NO: 370 - protein, 46 aa, synthetic construct
  REGION 1..46: note = Synthetic peptide
GAPGGGGAGGGGAGGGGAGGGGAGGGGAGGGPSGGGGAGGGGAGAP

SEQ ID NO: 371 - protein, 51 aa, synthetic construct
  REGION 1..51: note = Synthetic peptide
GAPGGGGAGGGGAGGGGAGGGGAGGGGAGGGPSGGGGAGGGGAGGGGAGAP

SEQ ID NO: 372 - protein, 56 aa, synthetic construct
  REGION 1..56: note = Synthetic peptide
GAPGGGGAGGGGAGGGGAGGGGAGGGGAGGGPSGGGGAGGGGAGGGGAGGGGAGAP

SEQ ID NO: 373 - protein, 61 aa, synthetic construct
  REGION 1..61: note = Synthetic peptide
GAPGGGGAGGGGAGGGGAGGGGAGGGGAGGGPSGGGGAGGGGAGGGGAGGGGAGGGGAGAP

SEQ ID NO: 374 - protein, 66 aa, synthetic construct
  REGION 1..66: note = Synthetic peptide
GAPGGGGAGGGGAGGGGAGGGGAGGGGAGGGPSGGGGAGGGGAGGGGAGGGGAGGGGAGGGGAGAP

SEQ ID NO: 375 - protein, 71 aa, synthetic construct
  REGION 1..71: note = Synthetic peptide
GAPGGGGAGGGGAGGGGAGGGGAGGGGAGGGPSGGGGAGGGGAGGGGAGGGGAGGGGAGGGGAGGGGAGAP

SEQ ID NO: 376 - protein, 46 aa, synthetic construct
  REGION 1..46: note = Synthetic peptide
GAPGGGGAGGGGAGGGGAGGGGAGGGGAGGGGAGGGPSGGGGAGAP

SEQ ID NO: 377 - protein, 51 aa, synthetic construct
  REGION 1..51: note = Synthetic peptide
GAPGGGGAGGGGAGGGGAGGGGAGGGGAGGGGAGGGPSGGGGAGGGGAGAP

SEQ ID NO: 378 - protein, 56 aa, synthetic construct
  REGION 1..56: note = Synthetic peptide
GAPGGGGAGGGGAGGGGAGGGGAGGGGAGGGGAGGGPSGGGGAGGGGAGGGGAGAP

SEQ ID NO: 379 - protein, 61 aa, synthetic construct
  REGION 1..61: note = Synthetic peptide
GAPGGGGAGGGGAGGGGAGGGGAGGGGAGGGGAGGGPSGGGGAGGGGAGGGGAGGGGAGAP

SEQ ID NO: 380 - protein, 66 aa, synthetic construct
  REGION 1..66: note = Synthetic peptide
GAPGGGGAGGGGAGGGGAGGGGAGGGGAGGGGAGGGPSGGGGAGGGGAGGGGAGGGGAGGGGAGAP

SEQ ID NO: 381 - protein, 71 aa, synthetic construct
  REGION 1..71: note = Synthetic peptide
GAPGGGGAGGGGAGGGGAGGGGAGGGGAGGGGAGGGPSGGGGAGGGGAGGGGAGGGGAGGGGAGGGGAGAP

SEQ ID NO: 382 - protein, 76 aa, synthetic construct
  REGION 1..76: note = Synthetic peptide
GAPGGGGAGGGGAGGGGAGGGGAGGGGAGGGGAGGGPSGGGGAGGGGAGGGGAGGGGAGGGGAGGGGAGGGGAGAP

SEQ ID NO: 383 - protein, 51 aa, synthetic construct
  REGION 1..51: note = Synthetic peptide
GAPGGGGAGGGGAGGGGAGGGGAGGGGAGGGGAGGGGAGGGPSGGGGAGAP

SEQ ID NO: 384 - protein, 56 aa, synthetic construct
  REGION 1..56: note = Synthetic peptide
GAPGGGGAGGGGAGGGGAGGGGAGGGGAGGGGAGGGGAGGGPSGGGGAGGGGAGAP

SEQ ID NO: 385 - protein, 61 aa, synthetic construct
  REGION 1..61: note = Synthetic peptide
GAPGGGGAGGGGAGGGGAGGGGAGGGGAGGGGAGGGGAGGGPSGGGGAGGGGAGGGGAGAP

SEQ ID NO: 386 - protein, 66 aa, synthetic construct
  REGION 1..66: note = Synthetic peptide
GAPGGGGAGGGGAGGGGAGGGGAGGGGAGGGGAGGGGAGGGPSGGGGAGGGGAGGGGAGGGGAGAP

SEQ ID NO: 387 - protein, 71 aa, synthetic construct
  REGION 1..71: note = Synthetic peptide
GAPGGGGAGGGGAGGGGAGGGGAGGGGAGGGGAGGGGAGGGPSGGGGAGGGGAGGGGAGGGGAGGGGAGAP

SEQ ID NO: 388 - protein, 76 aa, synthetic construct
  REGION 1..76: note = Synthetic peptide
GAPGGGGAGGGGAGGGGAGGGGAGGGGAGGGGAGGGGAGGGPSGGGGAGGGGAGGGGAGGGGAGGGGAGGGGAGAP

SEQ ID NO: 389 - protein, 81 aa, synthetic construct
  REGION 1..81: note = Synthetic peptide
GAPGGGGAGGGGAGGGGAGGGGAGGGGAGGGGAGGGGAGGGPSGGGGAGGGGAGGGGAGGGGAGGGGAGGGGAGGGGAGAP

SEQ ID NO: 390 - protein, 16 aa, synthetic construct
  REGION 1..16: note = Synthetic peptide
GAPGGGPSGGGGAGAP

SEQ ID NO: 391 - protein, 21 aa, synthetic construct
  REGION 1..21: note = Synthetic peptide
GAPGGGPSGGGGAGGGGAGAP

SEQ ID NO: 392 - protein, 26 aa, synthetic construct
  REGION 1..26: note = Synthetic peptide
GAPGGGPSGGGGAGGGGAGGGGAGAP

SEQ ID NO: 393 - protein, 31 aa, synthetic construct
  REGION 1..31: note = Synthetic peptide
GAPGGGPSGGGGAGGGGAGGGGAGGGGAGAP

SEQ ID NO: 394 - protein, 36 aa, synthetic construct
  REGION 1..36: note = Synthetic peptide
GAPGGGPSGGGGAGGGGAGGGGAGGGGAGGGGAGAP

SEQ ID NO: 395 - protein, 41 aa, synthetic construct
  REGION 1..41: note = Synthetic peptide
GAPGGGPSGGGGAGGGGAGGGGAGGGGAGGGGAGGGGAGAP

SEQ ID NO: 396 - protein, 46 aa, synthetic construct
  REGION 1..46: note = Synthetic peptide
GAPGGGPSGGGGAGGGGAGGGGAGGGGAGGGGAGGGGAGGGGAGAP

SEQ ID NO: 397 - protein, 30 aa, synthetic construct
  REGION 1..30: note = Synthetic peptide
GAPGGGGAAAAASGGGPSGGGGAAAAAGAP

SEQ ID NO: 398 - protein, 35 aa, synthetic construct
  REGION 1..35: note = Synthetic peptide
GAPGGGGAAAAASGGGPSGGGGAGGGGAAAAAGAP

SEQ ID NO: 399 - protein, 40 aa, synthetic construct
  REGION 1..40: note = Synthetic peptide
GAPGGGGAAAAASGGGPSGGGGAGGGGAGGGGAAAAAGAP

SEQ ID NO: 400 - protein, 45 aa, synthetic construct
  REGION 1..45: note = Synthetic peptide
GAPGGGGAAAAASGGGPSGGGGAGGGGAGGGGAGGGGAAAAAGAP

SEQ ID NO: 401 - protein, 50 aa, synthetic construct
  REGION 1..50: note = Synthetic peptide
GAPGGGGAAAAASGGGPSGGGGAGGGGAGGGGAGGGGAGGGGAAAAAGAP

SEQ ID NO: 402 - protein, 55 aa, synthetic construct
  REGION 1..55: note = Synthetic peptide
GAPGGGGAAAAASGGGPSGGGGAGGGGAGGGGAGGGGAGGGGAGGGGAAAAAGAP

SEQ ID NO: 403 - protein, 60 aa, synthetic construct
  REGION 1..60: note = Synthetic peptide
GAPGGGGAAAAASGGGPSGGGGAGGGGAGGGGAGGGGAGGGGAGGGGAGGGGAAAAAGAP

SEQ ID NO: 404 - protein, 35 aa, synthetic construct
  REGION 1..35: note = Synthetic peptide
GAPGGGGAGGGGAAAAASGGGPSGGGGAAAAAGAP

SEQ ID NO: 405 - protein, 40 aa, synthetic construct
  REGION 1..40: note = Synthetic peptide
GAPGGGGAGGGGAAAAASGGGPSGGGGAGGGGAAAAAGAP

SEQ ID NO: 406 - protein, 45 aa, synthetic construct
  REGION 1..45: note = Synthetic peptide
GAPGGGGAGGGGAAAAASGGGPSGGGGAGGGGAGGGGAAAAAGAP

SEQ ID NO: 407 - protein, 50 aa, synthetic construct
  REGION 1..50: note = Synthetic peptide
GAPGGGGAGGGGAAAAASGGGPSGGGGAGGGGAGGGGAGGGGAAAAAGAP

SEQ ID NO: 408 - protein, 55 aa, synthetic construct
  REGION 1..55: note = Synthetic peptide
GAPGGGGAGGGGAAAAASGGGPSGGGGAGGGGAGGGGAGGGGAGGGGAAAAAGAP

SEQ ID NO: 409 - protein, 60 aa, synthetic construct
  REGION 1..60: note = Synthetic peptide
GAPGGGGAGGGGAAAAASGGGPSGGGGAGGGGAGGGGAGGGGAGGGGAGGGGAAAAAGAP

SEQ ID NO: 410 - protein, 65 aa, synthetic construct
  REGION 1..65: note = Synthetic peptide
GAPGGGGAGGGGAAAAASGGGPSGGGGAGGGGAGGGGAGGGGAGGGGAGGGGAGGGGAAAAAGAP

SEQ ID NO: 411 - protein, 40 aa, synthetic construct
  REGION 1..40: note = Synthetic peptide
GAPGGGGAGGGGAGGGGAAAAASGGGPSGGGGAAAAAGAP

SEQ ID NO: 412 - protein, 45 aa, synthetic construct
  REGION 1..45: note = Synthetic peptide
GAPGGGGAGGGGAGGGGAAAAASGGGPSGGGGAGGGGAAAAAGAP

SEQ ID NO: 413 - protein, 50 aa, synthetic construct
  REGION 1..50: note = Synthetic peptide
GAPGGGGAGGGGAGGGGAAAAASGGGPSGGGGAGGGGAGGGGAAAAAGAP

SEQ ID NO: 414 - protein, 55 aa, synthetic construct
  REGION 1..55: note = Synthetic peptide
GAPGGGGAGGGGAGGGGAAAAASGGGPSGGGGAGGGGAGGGGAGGGGAAAAAGAP

SEQ ID NO: 415 - protein, 60 aa, synthetic construct
  REGION 1..60: note = Synthetic peptide
GAPGGGGAGGGGAGGGGAAAAASGGGPSGGGGAGGGGAGGGGAGGGGAGGGGAAAAAGAP

SEQ ID NO: 416 - protein, 65 aa, synthetic construct
  REGION 1..65: note = Synthetic peptide
GAPGGGGAGGGGAGGGGAAAAASGGGPSGGGGAGGGGAGGGGAGGGGAGGGGAGGGGAAAAAGAP

SEQ ID NO: 417 - protein, 70 aa, synthetic construct
  REGION 1..70: note = Synthetic peptide
GAPGGGGAGGGGAGGGGAAAAASGGGPSGGGGAGGGGAGGGGAGGGGAGGGGAGGGGAGGGGAAAAAGAP

SEQ ID NO: 418 - protein, 45 aa, synthetic construct
  REGION 1..45: note = Synthetic peptide
GAPGGGGAGGGGAGGGGAGGGGAAAAASGGGPSGGGGAAAAAGAP

SEQ ID NO: 419 - protein, 50 aa, synthetic construct
  REGION 1..50: note = Synthetic peptide
GAPGGGGAGGGGAGGGGAGGGGAAAAASGGGPSGGGGAGGGGAAAAAGAP

SEQ ID NO: 420 - protein, 55 aa, synthetic construct
  REGION 1..55: note = Synthetic peptide
GAPGGGGAGGGGAGGGGAGGGGAAAAASGGGPSGGGGAGGGGAGGGGAAAAAGAP

SEQ ID NO: 421 - protein, 60 aa, synthetic construct
  REGION 1..60: note = Synthetic peptide
GAPGGGGAGGGGAGGGGAGGGGAAAAASGGGPSGGGGAGGGGAGGGGAGGGGAAAAAGAP

SEQ ID NO: 422 - protein, 65 aa, synthetic construct
  REGION 1..65: note = Synthetic peptide
GAPGGGGAGGGGAGGGGAGGGGAAAAASGGGPSGGGGAGGGGAGGGGAGGGGAGGGGAAAAAGAP

SEQ ID NO: 423 - protein, 70 aa, synthetic construct
  REGION 1..70: note = Synthetic peptide
GAPGGGGAGGGGAGGGGAGGGGAAAAASGGGPSGGGGAGGGGAGGGGAGGGGAGGGGAGGGGAAAAAGAP

SEQ ID NO: 424 - protein, 75 aa, synthetic construct
  REGION 1..75: note = Synthetic peptide
GAPGGGGAGGGGAGGGGAGGGGAAAAASGGGPSGGGGAGGGGAGGGGAGGGGAGGGGAGGGGAGGGGAAAAAGAP

SEQ ID NO: 425 - protein, 50 aa, synthetic construct
  REGION 1..50: note = Synthetic peptide
GAPGGGGAGGGGAGGGGAGGGGAGGGGAAAAASGGGPSGGGGAAAAAGAP

SEQ ID NO: 426 - protein, 55 aa, synthetic construct
  REGION 1..55: note = Synthetic peptide
GAPGGGGAGGGGAGGGGAGGGGAGGGGAAAAASGGGPSGGGGAGGGGAAAAAGAP

SEQ ID NO: 427 - protein, 60 aa, synthetic construct
  REGION 1..60: note = Synthetic peptide
GAPGGGGAGGGGAGGGGAGGGGAGGGGAAAAASGGGPSGGGGAGGGGAGGGGAAAAAGAP

SEQ ID NO: 428 - protein, 65 aa, synthetic construct
  REGION 1..65: note = Synthetic peptide
GAPGGGGAGGGGAGGGGAGGGGAGGGGAAAAASGGGPSGGGGAGGGGAGGGGAGGGGAAAAAGAP

SEQ ID NO: 429 - protein, 70 aa, synthetic construct
  REGION 1..70: note = Synthetic peptide
GAPGGGGAGGGGAGGGGAGGGGAGGGGAAAAASGGGPSGGGGAGGGGAGGGGAGGGGAGGGGAAAAAGAP

SEQ ID NO: 430 - protein, 75 aa, synthetic construct
  REGION 1..75: note = Synthetic peptide
GAPGGGGAGGGGAGGGGAGGGGAGGGGAAAAASGGGPSGGGGAGGGGAGGGGAGGGGAGGGGAGGGGAAAAAGAP

SEQ ID NO: 431 - protein, 80 aa, synthetic construct
  REGION 1..80: note = Synthetic peptide
GAPGGGGAGGGGAGGGGAGGGGAGGGGAAAAASGGGPSGGGGAGGGGAGGGGAGGGGAGGGGAGGGGAGGGGAAAAAGAP

SEQ ID NO: 432 - protein, 55 aa, synthetic construct
  REGION 1..55: note = Synthetic peptide
GAPGGGGAGGGGAGGGGAGGGGAGGGGAGGGGAAAAASGGGPSGGGGAAAAAGAP

SEQ ID NO: 433 - protein, 60 aa, synthetic construct
  REGION 1..60: note = Synthetic peptide
GAPGGGGAGGGGAGGGGAGGGGAGGGGAGGGGAAAAASGGGPSGGGGAGGGGAAAAAGAP

SEQ ID NO: 434 - protein, 65 aa, synthetic construct
  REGION 1..65: note = Synthetic peptide
GAPGGGGAGGGGAGGGGAGGGGAGGGGAGGGGAAAAASGGGPSGGGGAGGGGAGGGGAAAAAGAP

SEQ ID NO: 435 - protein, 70 aa, synthetic construct
  REGION 1..70: note = Synthetic peptide
GAPGGGGAGGGGAGGGGAGGGGAGGGGAGGGGAAAAASGGGPSGGGGAGGGGAGGGGAGGGGAAAAAGAP

SEQ ID NO: 436 - protein, 75 aa, synthetic construct
  REGION 1..75: note = Synthetic peptide
GAPGGGGAGGGGAGGGGAGGGGAGGGGAGGGGAAAAASGGGPSGGGGAGGGGAGGGGAGGGGAGGGGAAAAAGAP

SEQ ID NO: 437 - protein, 80 aa, synthetic construct
  REGION 1..80: note = Synthetic peptide
GAPGGGGAGGGGAGGGGAGGGGAGGGGAGGGGAAAAASGGGPSGGGGAGGGGAGGGGAGGGGAGGGGAGGGGAAAAAGAP

SEQ ID NO: 438 - protein, 85 aa, synthetic construct
  REGION 1..85: note = Synthetic peptide
GAPGGGGAGGGGAGGGGAGGGGAGGGGAGGGGAAAAASGGGPSGGGGAGGGGAGGGGAGGGGAGGGGAGGGGAGGGGAAAAAGAP

SEQ ID NO: 439 - protein, 60 aa, synthetic construct
  REGION 1..60: note = Synthetic peptide
GAPGGGGAGGGGAGGGGAGGGGAGGGGAGGGGAGGGGAAAAASGGGPSGGGGAAAAAGAP

SEQ ID NO: 440 - protein, 65 aa, synthetic construct
  REGION 1..65: note = Synthetic peptide
GAPGGGGAGGGGAGGGGAGGGGAGGGGAGGGGAGGGGAAAAASGGGPSGGGGAGGGGAAAAAGAP

SEQ ID NO: 441 - protein, 70 aa, synthetic construct
  REGION 1..70: note = Synthetic peptide
GAPGGGGAGGGGAGGGGAGGGGAGGGGAGGGGAGGGGAAAAASGGGPSGGGGAGGGGAGGGGAAAAAGAP

SEQ ID NO: 442 - protein, 75 aa, synthetic construct
  REGION 1..75: note = Synthetic peptide
GAPGGGGAGGGGAGGGGAGGGGAGGGGAGGGGAGGGGAAAAASGGGPSGGGGAGGGGAGGGGAGGGGAAAAAGAP

SEQ ID NO: 443 - protein, 80 aa, synthetic construct
  REGION 1..80: note = Synthetic peptide
GAPGGGGAGGGGAGGGGAGGGGAGGGGAGGGGAGGGGAAAAASGGGPSGGGGAGGGGAGGGGAGGGGAGGGGAAAAAGAP

SEQ ID NO: 444 - protein, 85 aa, synthetic construct
  REGION 1..85: note = Synthetic peptide
GAPGGGGAGGGGAGGGGAGGGGAGGGGAGGGGAGGGGAAAAASGGGPSGGGGAGGGGAGGGGAGGGGAGGGGAGGGGAAAAAGAP

SEQ ID NO: 445 - protein, 90 aa, synthetic construct
  REGION 1..90: note = Synthetic peptide
GAPGGGGAGGGGAGGGGAGGGGAGGGGAGGGGAGGGGAAAAASGGGPSGGGGAGGGGAGGGGAGGGGAGGGGAGGGGAGGGGAAAAAGAP

SEQ ID NO: 446 - protein, 17 aa, synthetic construct
  REGION 1..17: note = Synthetic peptide
GAPGGGGAPAPAPXGAP

SEQ ID NO: 447 - protein, 18 aa, synthetic construct
  REGION 1..18: note = Synthetic peptide
GAPGGGGAPAPAPXXGAP

SEQ ID NO: 448 - protein, 19 aa, synthetic construct
  REGION 1..19: note = Synthetic peptide
GAPGGGGAPAPAPXXXGAP

SEQ ID NO: 449 - protein, 20 aa, synthetic construct
  REGION 1..20: note = Synthetic peptide
GAPGGGGAPAPAPXXXXGAP

SEQ ID NO: 450 - protein, 21 aa, synthetic construct
  REGION 1..21: note = Synthetic peptide
GAPGGGGAPAPAPXXXXXGAP

SEQ ID NO: 451 - protein, 22 aa, synthetic construct
  REGION 1..22: note = Synthetic peptide
GAPGGGGAPAPAPXXXXXXGAP

SEQ ID NO: 452 - protein, 23 aa, synthetic construct
  REGION 1..23: note = Synthetic peptide
GAPGGGGAPAPAPXXXXXXXGAP

SEQ ID NO: 453 - protein, 22 aa, synthetic construct
  REGION 1..22: note = Synthetic peptide
GAPGGGGAGGGGAPAPAPXGAP

SEQ ID NO: 454 - protein, 23 aa, synthetic construct
  REGION 1..23: note = Synthetic peptide
GAPGGGGAGGGGAPAPAPXXGAP

SEQ ID NO: 455 - protein, 24 aa, synthetic construct
  REGION 1..24: note = Synthetic peptide
GAPGGGGAGGGGAPAPAPXXXGAP

SEQ ID NO: 456 - protein, 25 aa, synthetic construct
  REGION 1..25: note = Synthetic peptide
GAPGGGGAGGGGAPAPAPXXXXGAP

SEQ ID NO: 457 - protein, 26 aa, synthetic construct
  REGION 1..26: note = Synthetic peptide
GAPGGGGAGGGGAPAPAPXXXXXGAP

SEQ ID NO: 458 - protein, 27 aa, synthetic construct
  REGION 1..27: note = Synthetic peptide
GAPGGGGAGGGGAPAPAPXXXXXXGAP

SEQ ID NO: 459 - protein, 28 aa, synthetic construct
  REGION 1..28: note = Synthetic peptide
GAPGGGGAGGGGAPAPAPXXXXXXXGAP

SEQ ID NO: 460 - protein, 27 aa, synthetic construct
  REGION 1..27: note = Synthetic peptide
GAPGGGGAGGGGAGGGGAPAPAPXGAP

SEQ ID NO: 461 - protein, 28 aa, synthetic construct
  REGION 1..28: note = Synthetic peptide
GAPGGGGAGGGGAGGGGAPAPAPXXGAP

SEQ ID NO: 462 - protein, 29 aa, synthetic construct
  REGION 1..29: note = Synthetic peptide
GAPGGGGAGGGGAGGGGAPAPAPXXXGAP

SEQ ID NO: 463 - protein, 30 aa, synthetic construct
  REGION 1..30: note = Synthetic peptide
GAPGGGGAGGGGAGGGGAPAPAPXXXXGAP

SEQ ID NO: 464 - protein, 31 aa, synthetic construct
  REGION 1..31: note = Synthetic peptide
GAPGGGGAGGGGAGGGGAPAPAPXXXXXGAP

SEQ ID NO: 465 - protein, 32 aa, synthetic construct
  REGION 1..32: note = Synthetic peptide
GAPGGGGAGGGGAGGGGAPAPAPXXXXXXGAP

SEQ ID NO: 466 - protein, 33 aa, synthetic construct
  REGION 1..33: note = Synthetic peptide
GAPGGGGAGGGGAGGGGAPAPAPXXXXXXXGAP

SEQ ID NO: 467 - protein, 32 aa, synthetic construct
  REGION 1..32: note = Synthetic peptide
GAPGGGGAGGGGAGGGGAGGGGAPAPAPXGAP

SEQ ID NO: 468 - protein, 33 aa, synthetic construct
  REGION 1..33: note = Synthetic peptide
GAPGGGGAGGGGAGGGGAGGGGAPAPAPXXGAP

SEQ ID NO: 469 - protein, 34 aa, synthetic construct
  REGION 1..34: note = Synthetic peptide
GAPGGGGAGGGGAGGGGAGGGGAPAPAPXXXGAP

SEQ ID NO: 470 - protein, 35 aa, synthetic construct
  REGION 1..35: note = Synthetic peptide
GAPGGGGAGGGGAGGGGAGGGGAPAPAPXXXXGAP

SEQ ID NO: 471 - protein, 36 aa, synthetic construct
  REGION 1..36: note = Synthetic peptide
GAPGGGGAGGGGAGGGGAGGGGAPAPAPXXXXXGAP

SEQ ID NO: 472 - protein, 37 aa, synthetic construct
  REGION 1..37: note = Synthetic peptide
GAPGGGGAGGGGAGGGGAGGGGAPAPAPXXXXXXGAP

SEQ ID NO: 473 - protein, 38 aa, synthetic construct
  REGION 1..38: note = Synthetic peptide
GAPGGGGAGGGGAGGGGAGGGGAPAPAPXXXXXXXGAP

SEQ ID NO: 474 - protein, 37 aa, synthetic construct
  REGION 1..37: note = Synthetic peptide
GAPGGGGAGGGGAGGGGAGGGGAGGGGAPAPAPXGAP

SEQ ID NO: 475 - protein, 38 aa, synthetic construct
  REGION 1..38: note = Synthetic peptide
GAPGGGGAGGGGAGGGGAGGGGAGGGGAPAPAPXXGAP

SEQ ID NO: 476 - protein, 39 aa, synthetic construct
  REGION 1..39: note = Synthetic peptide
GAPGGGGAGGGGAGGGGAGGGGAGGGGAPAPAPXXXGAP

SEQ ID NO: 477 - protein, 40 aa, synthetic construct
  REGION 1..40: note = Synthetic peptide
GAPGGGGAGGGGAGGGGAGGGGAGGGGAPAPAPXXXXGAP

SEQ ID NO: 478 - protein, 41 aa, synthetic construct
  REGION 1..41: note = Synthetic peptide
GAPGGGGAGGGGAGGGGAGGGGAGGGGAPAPAPXXXXXGAP

SEQ ID NO: 479 - protein, 42 aa, synthetic construct
  REGION 1..42: note = Synthetic peptide
GAPGGGGAGGGGAGGGGAGGGGAGGGGAPAPAPXXXXXXGAP

SEQ ID NO: 480 - protein, 43 aa, synthetic construct
  REGION 1..43: note = Synthetic peptide
GAPGGGGAGGGGAGGGGAGGGGAGGGGAPAPAPXXXXXXXGAP

SEQ ID NO: 481 - protein, 42 aa, synthetic construct
  REGION 1..42: note = Synthetic peptide
GAPGGGGAGGGGAGGGGAGGGGAGGGGAGGGGAPAPAPXGAP

SEQ ID NO: 482 - protein, 43 aa, synthetic construct
  REGION 1..43: note = Synthetic peptide
GAPGGGGAGGGGAGGGGAGGGGAGGGGAGGGGAPAPAPXXGAP

SEQ ID NO: 483 - protein, 44 aa, synthetic construct
  REGION 1..44: note = Synthetic peptide
GAPGGGGAGGGGAGGGGAGGGGAGGGGAGGGGAPAPAPXXXGAP

SEQ ID NO: 484 - protein, 45 aa, synthetic construct
  REGION 1..45: note = Synthetic peptide
GAPGGGGAGGGGAGGGGAGGGGAGGGGAGGGGAPAPAPXXXXGAP

SEQ ID NO: 485 - protein, 46 aa, synthetic construct
  REGION 1..46: note = Synthetic peptide
GAPGGGGAGGGGAGGGGAGGGGAGGGGAGGGGAPAPAPXXXXXGAP

SEQ ID NO: 486 - protein, 47 aa, synthetic construct
  REGION 1..47: note = Synthetic peptide
GAPGGGGAGGGGAGGGGAGGGGAGGGGAGGGGAPAPAPXXXXXXGAP

SEQ ID NO: 487 - protein, 48 aa, synthetic construct
  REGION 1..48: note = Synthetic peptide
GAPGGGGAGGGGAGGGGAGGGGAGGGGAGGGGAPAPAPXXXXXXXGAP

SEQ ID NO: 488 - protein, 47 aa, synthetic construct
  REGION 1..47: note = Synthetic peptide
GAPGGGGAGGGGAGGGGAGGGGAGGGGAGGGGAGGGGAPAPAPXGAP

SEQ ID NO: 489 - protein, 48 aa, synthetic construct
  REGION 1..48: note = Synthetic peptide
GAPGGGGAGGGGAGGGGAGGGGAGGGGAGGGGAGGGGAPAPAPXXGAP

SEQ ID NO: 490 - protein, 49 aa, synthetic construct
  REGION 1..49: note = Synthetic peptide
GAPGGGGAGGGGAGGGGAGGGGAGGGGAGGGGAGGGGAPAPAPXXXGAP

SEQ ID NO: 491 - protein, 50 aa, synthetic construct
  REGION 1..50: note = Synthetic peptide
GAPGGGGAGGGGAGGGGAGGGGAGGGGAGGGGAGGGGAPAPAPXXXXGAP

SEQ ID NO: 492 - protein, 51 aa, synthetic construct
  REGION 1..51: note = Synthetic peptide
GAPGGGGAGGGGAGGGGAGGGGAGGGGAGGGGAGGGGAPAPAPXXXXXGAP

SEQ ID NO: 493 - protein, 52 aa, synthetic construct
  REGION 1..52: note = Synthetic peptide
GAPGGGGAGGGGAGGGGAGGGGAGGGGAGGGGAGGGGAPAPAPXXXXXXGAP

SEQ ID NO: 494 - protein, 53 aa, synthetic construct
  REGION 1..53: note = Synthetic peptide
GAPGGGGAGGGGAGGGGAGGGGAGGGGAGGGGAGGGGAPAPAPXXXXXXXGAP

SEQ ID NO: 495 - protein, 18 aa, synthetic construct
  REGION 1..18: note = Synthetic peptide
GAPGGGGAPAPAPTXGAP

SEQ ID NO: 496 - protein, 19 aa, synthetic construct
  REGION 1..19: note = Synthetic peptide
GAPGGGGAPAPAPTXXGAP

SEQ ID NO: 497 - protein, 20 aa, synthetic construct
  REGION 1..20: note = Synthetic peptide
GAPGGGGAPAPAPTXXXGAP

SEQ ID NO: 498 - protein, 21 aa, synthetic construct
  REGION 1..21: note = Synthetic peptide
GAPGGGGAPAPAPTXXXXGAP

SEQ ID NO: 499 - protein, 22 aa, synthetic construct
  REGION 1..22: note = Synthetic peptide
GAPGGGGAPAPAPTXXXXXGAP

SEQ ID NO: 500 - protein, 23 aa, synthetic construct
  REGION 1..23: note = Synthetic peptide
GAPGGGGAPAPAPTXXXXXXGAP

SEQ ID NO: 501 - protein, 24 aa, synthetic construct
  REGION 1..24: note = Synthetic peptide
GAPGGGGAPAPAPTXXXXXXXGAP

SEQ ID NO: 502 - protein, 23 aa, synthetic construct
  REGION 1..23: note = Synthetic peptide
GAPGGGGAGGGGAPAPAPTXGAP

SEQ ID NO: 503 - protein, 24 aa, synthetic construct
  REGION 1..24: note = Synthetic peptide
GAPGGGGAGGGGAPAPAPTXXGAP

SEQ ID NO: 504 - protein, 25 aa, synthetic construct
  REGION 1..25: note = Synthetic peptide
GAPGGGGAGGGGAPAPAPTXXXGAP

SEQ ID NO: 505 - protein, 26 aa, synthetic construct
  REGION 1..26: note = Synthetic peptide
GAPGGGGAGGGGAPAPAPTXXXXGAP

SEQ ID NO: 506 - protein, 27 aa, synthetic construct
  REGION 1..27: note = Synthetic peptide
GAPGGGGAGGGGAPAPAPTXXXXXGAP

SEQ ID NO: 507 - protein, 28 aa, synthetic construct
  REGION 1..28: note = Synthetic peptide
GAPGGGGAGGGGAPAPAPTXXXXXXGAP

SEQ ID NO: 508 - protein, 29 aa, synthetic construct
  REGION 1..29: note = Synthetic peptide
GAPGGGGAGGGGAPAPAPTXXXXXXXGAP

SEQ ID NO: 509 - protein, 28 aa, synthetic construct
  REGION 1..28: note = Synthetic peptide
GAPGGGGAGGGGAGGGGAPAPAPTXGAP

SEQ ID NO: 510 - protein, 29 aa, synthetic construct
  REGION 1..29: note = Synthetic peptide
GAPGGGGAGGGGAGGGGAPAPAPTXXGAP

SEQ ID NO: 511 - protein, 30 aa, synthetic construct
  REGION 1..30: note = Synthetic peptide
GAPGGGGAGGGGAGGGGAPAPAPTXXXGAP

SEQ ID NO: 512 - protein, 31 aa, synthetic construct
  REGION 1..31: note = Synthetic peptide
GAPGGGGAGGGGAGGGGAPAPAPTXXXXGAP

SEQ ID NO: 513 - protein, 32 aa, synthetic construct
  REGION 1..32: note = Synthetic peptide
GAPGGGGAGGGGAGGGGAPAPAPTXXXXXGAP

SEQ ID NO: 514 - protein, 33 aa, synthetic construct
  REGION 1..33: note = Synthetic peptide
GAPGGGGAGGGGAGGGGAPAPAPTXXXXXXGAP

SEQ ID NO: 515 - protein, 34 aa, synthetic construct
  REGION 1..34: note = Synthetic peptide
GAPGGGGAGGGGAGGGGAPAPAPTXXXXXXXGAP

SEQ ID NO: 516 - protein, 33 aa, synthetic construct
  REGION 1..33: note = Synthetic peptide
GAPGGGGAGGGGAGGGGAGGGGAPAPAPTXGAP

SEQ ID NO: 517 - protein, 34 aa, synthetic construct
  REGION 1..34: note = Synthetic peptide
GAPGGGGAGGGGAGGGGAGGGGAPAPAPTXXGAP

SEQ ID NO: 518 - protein, 35 aa, synthetic construct
  REGION 1..35: note = Synthetic peptide
GAPGGGGAGGGGAGGGGAGGGGAPAPAPTXXXGAP

SEQ ID NO: 519 - protein, 36 aa, synthetic construct
  REGION 1..36: note = Synthetic peptide
GAPGGGGAGGGGAGGGGAGGGGAPAPAPTXXXXGAP

SEQ ID NO: 520 - protein, 37 aa, synthetic construct
  REGION 1..37: note = Synthetic peptide
GAPGGGGAGGGGAGGGGAGGGGAPAPAPTXXXXXGAP

SEQ ID NO: 521 - protein, 38 aa, synthetic construct
  REGION 1..38: note = Synthetic peptide
GAPGGGGAGGGGAGGGGAGGGGAPAPAPTXXXXXXGAP

SEQ ID NO: 522 - protein, 39 aa, synthetic construct
  REGION 1..39: note = Synthetic peptide
GAPGGGGAGGGGAGGGGAGGGGAPAPAPTXXXXXXXGAP

SEQ ID NO: 523 - protein, 38 aa, synthetic construct
  REGION 1..38: note = Synthetic peptide
GAPGGGGAGGGGAGGGGAGGGGAGGGGAPAPAPTXGAP

SEQ ID NO: 524 - protein, 39 aa, synthetic construct
  REGION 1..39: note = Synthetic peptide
GAPGGGGAGGGGAGGGGAGGGGAGGGGAPAPAPTXXGAP

SEQ ID NO: 525 - protein, 40 aa, synthetic construct
  REGION 1..40: note = Synthetic peptide
GAPGGGGAGGGGAGGGGAGGGGAGGGGAPAPAPTXXXGAP

SEQ ID NO: 526 - protein, 41 aa, synthetic construct
  REGION 1..41: note = Synthetic peptide
GAPGGGGAGGGGAGGGGAGGGGAGGGGAPAPAPTXXXXGAP

SEQ ID NO: 527 - protein, 42 aa, synthetic construct
  REGION 1..42: note = Synthetic peptide
GAPGGGGAGGGGAGGGGAGGGGAGGGGAPAPAPTXXXXXGAP

SEQ ID NO: 528 - protein, 43 aa, synthetic construct
  REGION 1..43: note = Synthetic peptide
GAPGGGGAGGGGAGGGGAGGGGAGGGGAPAPAPTXXXXXXGAP

SEQ ID NO: 529 - protein, 44 aa, synthetic construct
  REGION 1..44: note = Synthetic peptide
GAPGGGGAGGGGAGGGGAGGGGAGGGGAPAPAPTXXXXXXXGAP

SEQ ID NO: 530 - protein, 43 aa, synthetic construct
  REGION 1..43: note = Synthetic peptide
GAPGGGGAGGGGAGGGGAGGGGAGGGGAGGGGAPAPAPTXGAP

SEQ ID NO: 531 - protein, 44 aa, synthetic construct
  REGION 1..44: note = Synthetic peptide
GAPGGGGAGGGGAGGGGAGGGGAGGGGAGGGGAPAPAPTXXGAP

SEQ ID NO: 532 - protein, 45 aa, synthetic construct
  REGION 1..45: note = Synthetic peptide
GAPGGGGAGGGGAGGGGAGGGGAGGGGAGGGGAPAPAPTXXXGAP

SEQ ID NO: 533 - protein, 46 aa, synthetic construct
  REGION 1..46: note = Synthetic peptide
GAPGGGGAGGGGAGGGGAGGGGAGGGGAGGGGAPAPAPTXXXXGAP

SEQ ID NO: 534 - protein, 47 aa, synthetic construct
  REGION 1..47: note = Synthetic peptide
GAPGGGGAGGGGAGGGGAGGGGAGGGGAGGGGAPAPAPTXXXXXGAP

SEQ ID NO: 535 - protein, 48 aa, synthetic construct
  REGION 1..48: note = Synthetic peptide
GAPGGGGAGGGGAGGGGAGGGGAGGGGAGGGGAPAPAPTXXXXXXGAP

SEQ ID NO: 536 - protein, 49 aa, synthetic construct
  REGION 1..49: note = Synthetic peptide
GAPGGGGAGGGGAGGGGAGGGGAGGGGAGGGGAPAPAPTXXXXXXXGAP

SEQ ID NO: 537 - protein, 48 aa, synthetic construct
  REGION 1..48: note = Synthetic peptide
GAPGGGGAGGGGAGGGGAGGGGAGGGGAGGGGAGGGGAPAPAPTXGAP

SEQ ID NO: 538 - protein, 49 aa, synthetic construct
  REGION 1..49: note = Synthetic peptide
GAPGGGGAGGGGAGGGGAGGGGAGGGGAGGGGAGGGGAPAPAPTXXGAP

SEQ ID NO: 539 - protein, 50 aa, synthetic construct
  REGION 1..50: note = Synthetic peptide
GAPGGGGAGGGGAGGGGAGGGGAGGGGAGGGGAGGGGAPAPAPTXXXGAP

SEQ ID NO: 540 - protein, 51 aa, synthetic construct
  REGION 1..51: note = Synthetic peptide
GAPGGGGAGGGGAGGGGAGGGGAGGGGAGGGGAGGGGAPAPAPTXXXXGAP

SEQ ID NO: 541 - protein, 52 aa, synthetic construct
  REGION 1..52: note = Synthetic peptide
GAPGGGGAGGGGAGGGGAGGGGAGGGGAGGGGAGGGGAPAPAPTXXXXXGAP

SEQ ID NO: 542 - protein, 53 aa, synthetic construct
  REGION 1..53: note = Synthetic peptide
GAPGGGGAGGGGAGGGGAGGGGAGGGGAGGGGAGGGGAPAPAPTXXXXXXGAP

SEQ ID NO: 543 - protein, 54 aa, synthetic construct
  REGION 1..54: note = Synthetic peptide
GAPGGGGAGGGGAGGGGAGGGGAGGGGAGGGGAGGGGAPAPAPTXXXXXXXGAP

SEQ ID NO: 544 - protein, 29 aa, synthetic construct
  REGION 1..29: note = Synthetic peptide
GAPGGGGSXPAPAPXAAAKEXGGGGSGAP

SEQ ID NO: 545 - protein, 47 aa, synthetic construct
  REGION 1..47: note = Synthetic peptide
GAPGGGGSGGGGSXXPAPAPXXAAAKEAAAKEXXGGGGSGGGGSGAP

SEQ ID NO: 546 - protein, 65 aa, synthetic construct
  REGION 1..65: note = Synthetic peptide
GAPGGGGSGGGGSGGGGSXXXPAPAPXXXAAAKEAAAKEAAAKEXXXGGGGSGGGGSGGGGSGAP

SEQ ID NO: 547 - protein, 83 aa, synthetic construct
  REGION 1..83: note = Synthetic peptide
GAPGGGGSGGGGSGGGGSGGGGSXXXXPAPAPXXXXAAAKEAAAKEAAAKEAAAKEXXXXGGGGSGGGGSGGGGSGGGGSGAP

SEQ ID NO: 548 - protein, 101 aa, synthetic construct
  REGION 1..101: note = Synthetic peptide
GAPGGGGSGGGGSGGGGSGGGGSGGGGSXXXXXPAPAPXXXXXAAAKEAAAKEAAAKEAAAKEAAAKEXXXXXGGGGSGGGGSGGGGSGGGGSGGGGSGAP

SEQ ID NO: 549 - protein, 119 aa, synthetic construct
  REGION 1..119: note = Synthetic peptide
GAPGGGGSGGGGSGGGGSGGGGSGGGGSGGGGSXXXXXXPAPAPXXXXXXAAAKEAAAKEAAAKEAAAKEAAAKEAAAKEXXXXXXGGGGSGGGGSGGGGSGGGGSGGGGSGGGGSGAP

SEQ ID NO: 550 - protein, 137 aa, synthetic construct
  REGION 1..137: note = Synthetic peptide
GAPGGGGSGGGGSGGGGSGGGGSGGGGSGGGGSGGGGSXXXXXXXPAPAPXXXXXXXAAAKEAAAKEAAAKEAAAKEAAAKEAAAKEAAAKEXXXXXXXGGGGSGGGGSGGGGSGGGGSGGGGSGGGGSGGGGSGAP

SEQ ID NO: 551 - protein, 137 aa, synthetic construct; identical to SEQ ID NO: 550
  REGION 1..137: note = Synthetic peptide
  VARIANT 9..38: note = GGGGS repeats may be deleted
  VARIANT 39: note = Any amino acid
  VARIANT 40..45: note = Any amino acid or may be deleted
  VARIANT 51: note = Any amino acid
  VARIANT 52..57: note = Any amino acid or may be deleted
  VARIANT 63..92: note = AAAKE repeats may be deleted
  VARIANT 93: note = Any amino acid
  VARIANT 94..99: note = Any amino acid or may be deleted
  VARIANT 105..134: note = GGGGS repeats may be deleted

SEQ ID NO: 552 - protein, 29 aa, synthetic construct
  REGION 1..29: note = Synthetic peptide
GAPGGGGAXPAPAPXAAAKEXGGGGAGAP

SEQ ID NO: 553 - protein, 47 aa, synthetic construct
  REGION 1..47: note = Synthetic peptide
GAPGGGGAGGGGAXXPAPAPXXAAAKEAAAKEXXGGGGAGGGGAGAP

SEQ ID NO: 554 - protein, 65 aa, synthetic construct
  REGION 1..65: note = Synthetic peptide
GAPGGGGAGGGGAGGGGAXXXPAPAPXXXAAAKEAAAKEAAAKEXXXGGGGAGGGGAGGGGAGAP

SEQ ID NO: 555 - protein, 83 aa, synthetic construct
  REGION 1..83: note = Synthetic peptide
GAPGGGGAGGGGAGGGGAGGGGAXXXXPAPAPXXXXAAAKEAAAKEAAAKEAAAKEXXXXGGGGAGGGGAGGGGAGGGGAGAP

SEQ ID NO: 556 - protein, 101 aa, synthetic construct
  REGION 1..101: note = Synthetic peptide
GAPGGGGAGGGGAGGGGAGGGGAGGGGAXXXXXPAPAPXXXXXAAAKEAAAKEAAAKEAAAKEAAAKEXXXXXGGGGAGGGGAGGGGAGGGGAGGGGAGAP

SEQ ID NO: 557 - protein, 119 aa, synthetic construct
  REGION 1..119: note = Synthetic peptide
GAPGGGGAGGGGAGGGGAGGGGAGGGGAGGGGAXXXXXXPAPAPXXXXXXAAAKEAAAKEAAAKEAAAKEAAAKEAAAKEXXXXXXGGGGAGGGGAGGGGAGGGGAGGGGAGGGGAGAP

SEQ ID NO: 558 - protein, 137 aa, synthetic construct
  REGION 1..137: note = Synthetic peptide
GAPGGGGAGGGGAGGGGAGGGGAGGGGAGGGGAGGGGAXXXXXXXPAPAPXXXXXXXAAAKEAAAKEAAAKEAAAKEAAAKEAAAKEAAAKEXXXXXXXGGGGAGGGGAGGGGAGGGGAGGGGAGGGGAGGGGAGAP

SEQ ID NO: 559 - protein, 137 aa, synthetic construct; identical to SEQ ID NO: 558
  REGION 1..137: note = Synthetic peptide
  VARIANT 9..38: note = GGGGA repeats may be deleted
  VARIANT 39: note = Any amino acid
  VARIANT 40..45: note = Any amino acid or may be deleted
  VARIANT 51: note = Any amino acid
  VARIANT 52..57: note = Any amino acid or may be deleted
  VARIANT 63..92: note = AAAKE repeats may be deleted
  VARIANT 93: note = Any amino acid
  VARIANT 94..99: note = Any amino acid or may be deleted
  VARIANT 105..134: note = GGGGA repeats may be deleted

SEQ ID NO: 560 - protein, 812 aa, synthetic construct
  REGION 1..812: note = Synthetic peptide
MEAVAVAAAVGVLLLAGAGGAAGDEAREAAAVRALVARLLGPGPAADFSVSVERALAAKPGLDTYSLGGGGAARVRVRGSTGVAAAAGLHRYLRDFCGCHVAWSGSQLRLPRPLPAVPGELTEATPNRYRYYQNVCTQSYSFVWWDWARWEREIDWMALNGINLALAWSGQEAIWQRVYLALGLTQAEINEFFTGPAFLAWGRMGNLHTWDGPLPPSWHIKQLYLQHRVLDQMRSFGMTPVLPAFAGHVPEAVTRVFPQVNVTKMGSWGHFNCSYSCSFLLAPEDPIFPIIGSLFLRELIKEFGTDHIYGADTFNEMQPPSSEPSYLAAATTAVYEAMTAVDTEAVWLLQGWLFQHQPQFWGPAQIRAVLGAVPRGRLLVLDLFAESQPVYTRTASFQGQPFIWCMLHNFGGNHGLFGALEAVNGGPEAARLFPNSTMVGTGMAPEGISQNEVVYSLMAELGWRKDPVPDLAAWVTSFAARRYGVSHPDAGAAWRLLLRSVYNCSGEACRGHNRSPLVRRPSLQMNTSIWYNRSDVFEAWRLLLTSAPSLATSPAFRYDLLDLTRQAVQELVSLYYEEARSAYLSKELASLLRAGGVLAYELLPALDEVLASDSRFLLGSWLEQARAAAVSEAEADFYEQNSRYQLTLWGPEGNILDYANKQLAGLVANYYTPRWRLFLEALVDSVAQGIPFQQHQFDKNVFQLEQAFVLSKQRYPSQPRGDTVDLAKKIFLKYYPRWVAGSWEFGGGGSTRLCGGELVDTLQFVCGDRGFYFSRPASRVSARSRGIVEECCFRSCDLALLETYCATPAKSE

SEQ ID NO: 561 - protein, 806 aa, synthetic construct
  REGION 1..806: note = Synthetic peptide
MEAVAVAAAVGVLLLAGAGGAAGDEAREAAAVRALVARLLGPGPAADFSVSVERALAAKPGLDTYSLGGGGAARVRVRGSTGVAAAAGLHRYLRDFCGCHVAWSGSQLRLPRPLPAVPGELTEATPNRYRYYQNVCTQSYSFVWWDWARWEREIDWMALNGINLALAWSGQEAIWQRVYLALGLTQAEINEFFTGPAFLAWGRMGNLHTWDGPLPPSWHIKQLYLQHRVLDQMRSFGMTPVLPAFAGHVPEAVTRVFPQVNVTKMGSWGHFNCSYSCSFLLAPEDPIFPIIGSLFLRELIKEFGTDHIYGADTFNEMQPPSSEPSYLAAATTAVYEAMTAVDTEAVWLLQGWLFQHQPQFWGPAQIRAVLGAVPRGRLLVLDLFAESQPVYTRTASFQGQPFIWCMLHNFGGNHGLFGALEAVNGGPEAARLFPNSTMVGTGMAPEGISQNEVVYSLMAELGWRKDPVPDLAAWVTSFAARRYGVSHPDAGAAWRLLLRSVYNCSGEACRGHNRSPLVRRPSLQMNTSIWYNRSDVFEAWRLLLTSAPSLATSPAFRYDLLDLTRQAVQELVSLYYEEARSAYLSKELASLLRAGGVLAYELLPALDEVLASDSRFLLGSWLEQARAAAVSEAEADFYEQNSRYQLTLWGPEGNILDYANKQLAGLVANYYTPRWRLFLEALVDSVAQGIPFQQHQFDKNVFQLEQAFVLSKQRYPSQPRGDTVDLAKKIFLKYYPRWVAGSWGAPLCGGELVDTLQFVCGDRGFYFSRPASRVSARSRGIVEECCFRSCDLALLETYCATPAKSE

SEQ ID NO: 562 - protein, 808 aa, synthetic construct
  REGION 1..808: note = Synthetic peptide
MEAVAVAAAVGVLLLAGAGGAAGDEAREAAAVRALVARLLGPGPAADFSVSVERALAAKPGLDTYSLGGGGAARVRVRGSTGVAAAAGLHRYLRDFCGCHVAWSGSQLRLPRPLPAVPGELTEATPNRYRYYQNVCTQSYSFVWWDWARWEREIDWMALNGINLALAWSGQEAIWQRVYLALGLTQAEINEFFTGPAFLAWGRMGNLHTWDGPLPPSWHIKQLYLQHRVLDQMRSFGMTPVLPAFAGHVPEAVTRVFPQVNVTKMGSWGHFNCSYSCSFLLAPEDPIFPIIGSLFLRELIKEFGTDHIYGADTFNEMQPPSSEPSYLAAATTAVYEAMTAVDTEAVWLLQGWLFQHQPQFWGPAQIRAVLGAVPRGRLLVLDLFAESQPVYTRTASFQGQPFIWCMLHNFGGNHGLFGALEAVNGGPEAARLFPNSTMVGTGMAPEGISQNEVVYSLMAELGWRKDPVPDLAAWVTSFAARRYGVSHPDAGAAWRLLLRSVYNCSGEACRGHNRSPLVRRPSLQMNTSIWYNRSDVFEAWRLLLTSAPSLATSPAFRYDLLDLTRQAVQELVSLYYEEARSAYLSKELASLLRAGGVLAYELLPALDEVLASDSRFLLGSWLEQARAAAVSEAEADFYEQNSRYQLTLWGPEGNILDYANKQLAGLVANYYTPRWRLFLEALVDSVAQGIPFQQHQFDKNVFQLEQAFVLSKQRYPSQPRGDTVDLAKKIFLKYYPRWVAGSWGGGGSLCGGELVDTLQFVCGDRGFYFSRPASRVSARSRGIVEECCFRSCDLALLETYCATPAKSE

SEQ ID NO: 563 - protein, 810 aa, synthetic construct
  REGION 1..810: note = Synthetic peptide
MEAVAVAAAVGVLLLAGAGGAAGDEAREAAAVRALVARLLGPGPAADFSVSVERALAAKPGLDTYSLGGGGAARVRVRGSTGVAAAAGLHRYLRDFCGCHVAWSGSQLRLPRPLPAVPGELTEATPNRYRYYQNVCTQSYSFVWWDWARWEREIDWMALNGINLALAWSGQEAIWQRVYLALGLTQAEINEFFTGPAFLAWGRMGNLHTWDGPLPPSWHIKQLYLQHRVLDQMRSFGMTPVLPAFAGHVPEAVTRVFPQVNVTKMGSWGHFNCSYSCSFLLAPEDPIFPIIGSLFLRELIKEFGTDHIYGADTFNEMQPPSSEPSYLAAATTAVYEAMTAVDTEAVWLLQGWLFQHQPQFWGPAQIRAVLGAVPRGRLLVLDLFAESQPVYTRTASFQGQPFIWCMLHNFGGNHGLFGALEAVNGGPEAARLFPNSTMVGTGMAPEGISQNEVVYSLMAELGWRKDPVPDLAAWVTSFAARRYGVSHPDAGAAWRLLLRSVYNCSGEACRGHNRSPLVRRPSLQMNTSIWYNRSDVFEAWRLLLTSAPSLATSPAFRYDLLDLTRQAVQELVSLYYEEARSAYLSKELASLLRAGGVLAYELLPALDEVLASDSRFLLGSWLEQARAAAVSEAEADFYEQNSRYQLTLWGPEGNILDYANKQLAGLVANYYTPRWRLFLEALVDSVAQGIPFQQHQFDKNVFQLEQAFVLSKQRYPSQPRGDTVDLAKKIFLKYYPRWVAGSWGPSGSPGLCGGELVDTLQFVCGDRGFYFSRPASRVSARSRGIVEECCFRSCDLALLETYCATPAKSE

SEQ ID NO: 564 - protein, 828 aa, synthetic construct
  REGION 1..828: note = Synthetic peptide
MEAVAVAAAVGVLLLAGAGGAAGDEAREAAAVRALVARLLGPGPAADFSVSVERALAAKPGLDTYSLGGGGAARVRVRGSTGVAAAAGLHRYLRDFCGCHVAWSGSQLRLPRPLPAVPGELTEATPNRYRYYQNVCTQSYSFVWWDWARWEREIDWMALNGINLALAWSGQEAIWQRVYLALGLTQAEINEFFTGPAFLAWGRMGNLHTWDGPLPPSWHIKQLYLQHRVLDQMRSFGMTPVLPAFAGHVPEAVTRVFPQVNVTKMGSWGHFNCSYSCSFLLAPEDPIFPIIGSLFLRELIKEFGTDHIYGADTFNEMQPPSSEPSYLAAATTAVYEAMTAVDTEAVWLLQGWLFQHQPQFWGPAQIRAVLGAVPRGRLLVLDLFAESQPVYTRTASFQGQPFIWCMLHNFGGNHGLFGALEAVNGGPEAARLFPNSTMVGTGMAPEGISQNEVVYSLMAELGWRKDPVPDLAAWVTSFAARRYGVSHPDAGAAWRLLLRSVYNCSGEACRGHNRSPLVRRPSLQMNTSIWYNRSDVFEAWRLLLTSAPSLATSPAFRYDLLDLTRQAVQELVSLYYEEARSAYLSKELASLLRAGGVLAYELLPALDEVLASDSRFLLGSWLEQARAAAVSEAEADFYEQNSRYQLTLWGPEGNILDYANKQLAGLVANYYTPRWRLFLEALVDSVAQGIPFQQHQFDKNVFQLEQAFVLSKQRYPSQPRGDTVDLAKKIFLKYYPRWVAGSWGGGGSGGGGSGGGGSGGGGSGGGPSLCGGELVDTLQFVCGDRGFYFSRPASRVSARSRGIVEECCFRSCDLALLETYCATPAKSE

SEQ ID NO: 565 - protein, 859 aa, synthetic construct
  REGION 1..859: note = Synthetic peptide
MEAVAVAAAVGVLLLAGAGGAAGDEAREAAAVRALVARLLGPGPAADFSVSVERALAAKPGLDTYSLGGGGAARVRVRGSTGVAAAAGLHRYLRDFCGCHVAWSGSQLRLPRPLPAVPGELTEATPNRYRYYQNVCTQSYSFVWWDWARWEREIDWMALNGINLALAWSGQEAIWQRVYLALGLTQAEINEFFTGPAFLAWGRMGNLHTWDGPLPPSWHIKQLYLQHRVLDQMRSFGMTPVLPAFAGHVPEAVTRVFPQVNVTKMGSWGHFNCSYSCSFLLAPEDPIFPIIGSLFLRELIKEFGTDHIYGADTFNEMQPPSSEPSYLAAATTAVYEAMTAVDTEAVWLLQGWLFQHQPQFWGPAQIRAVLGAVPRGRLLVLDLFAESQPVYTRTASFQGQPFIWCMLHNFGGNHGLFGALEAVNGGPEAARLFPNSTMVGTGMAPEGISQNEVVYSLMAELGWRKDPVPDLAAWVTSFAARRYGVSHPDAGAAWRLLLRSVYNCSGEACRGHNRSPLVRRPSLQMNTSIWYNRSDVFEAWRLLLTSAPSLATSPAFRYDLLDLTRQAVQELVSLYYEEARSAYLSKELASLLRAGGVLAYELLPALDEVLASDSRFLLGSWLEQARAAAVSEAEADFYEQNSRYQLTLWGPEGNILDYANKQLAGLVANYYTPRWRLFLEALVDSVAQGIPFQQHQFDKNVFQLEQAFVLSKQRYPSQPRGDTVDLAKKIFLKYYPRWVAGSWGAPGGSPAGSPTSTEEGTSESATPESGPGTSTEPSEGSAPGSPAGSPTSTGPSGAPLCGGELVDTLQFVCGDRGFYFSRPASRVSARSRGIVEECCFRSCDLALLETYCATPAKSE

SEQ ID NO: 566 - protein, 834 aa, synthetic construct
  REGION 1..834: note = Synthetic peptide
MEAVAVAAAVGVLLLAGAGGAAGDEAREAAAVRALVARLLGPGPAADFSVSVERALAAKPGLDTYSLGGGGAARVRVRGSTGVAAAAGLHRYLRDFCGCHVAWSGSQLRLPRPLPAVPGELTEATPNRYRYYQNVCTQSYSFVWWDWARWEREIDWMALNGINLALAWSGQEAIWQRVYLALGLTQAEINEFFTGPAFLAWGRMGNLHTWDGPLPPSWHIKQLYLQHRVLDQMRSFGMTPVLPAFAGHVPEAVTRVFPQVNVTKMGSWGHFNCSYSCSFLLAPEDPIFPIIGSLFLRELIKEFGTDHIYGADTFNEMQPPSSEPSYLAAATTAVYEAMTAVDTEAVWLLQGWLFQHQPQFWGPAQIRAVLGAVPRGRLLVLDLFAESQPVYTRTASFQGQPFIWCMLHNFGGNHGLFGALEAVNGGPEAARLFPNSTMVGTGMAPEGISQNEVVYSLMAELGWRKDPVPDLAAWVTSFAARRYGVSHPDAGAAWRLLLRSVYNCSGEACRGHNRSPLVRRPSLQMNTSIWYNRSDVFEAWRLLLTSAPSLATSPAFRYDLLDLTRQAVQELVSLYYEEARSAYLSKELASLLRAGGVLAYELLPALDEVLASDSRFLLGSWLEQARAAAVSEAEADFYEQNSRYQLTLWGPEGNILDYANKQLAGLVANYYTPRWRLFLEALVDSVAQGIPFQQHQFDKNVFQLEQAFVLSKQRYPSQPRGDTVDLAKKIFLKYYPRWVAGSWGAPGGGSPAPAPTPAPAPTPAPAGGGPSGAPLCGGELVDTLQFVCGDRGFYFSRPASRVSARSRGIVEECCFRSCDLALLETYCATPAKSE

SEQ ID NO: 567 - protein, 846 aa, synthetic construct
  REGION 1..846: note = Synthetic peptide
MEAVAVAAAVGVLLLAGAGGAAGDEAREAAAVRALVARLLGPGPAADFSVSVERALAAKPGLDTYSLGGGGAARVRVRGSTGVAAAAGLHRYLRDFCGCHVAWSGSQLRLPRPLPAVPGELTEATPNRYRYYQNVCTQSYSFVWWDWARWEREIDWMALNGINLALAWSGQEAIWQRVYLALGLTQAEINEFFTGPAFLAWGRMGNLHTWDGPLPPSWHIKQLYLQHRVLDQMRSFGMTPVLPAFAGHVPEAVTRVFPQVNVTKMGSWGHFNCSYSCSFLLAPEDPIFPIIGSLFLRELIKEFGTDHIYGADTFNEMQPPSSEPSYLAAATTAVYEAMTAVDTEAVWLLQGWLFQHQPQFWGPAQIRAVLGAVPRGRLLVLDLFAESQPVYTRTASFQGQPFIWCMLHNFGGNHGLFGALEAVNGGPEAARLFPNSTMVGTGMAPEGISQNEVVYSLMAELGWRKDPVPDLAAWVTSFAARRYGVSHPDAGAAWRLLLRSVYNCSGEACRGHNRSPLVRRPSLQMNTSIWYNRSDVFEAWRLLLTSAPSLATSPAFRYDLLDLTRQAVQELVSLYYEEARSAYLSKELASLLRAGGVLAYELLPALDEVLASDSRFLLGSWLEQARAAAVSEAEADFYEQNSRYQLTLWGPEGNILDYANKQLAGLVANYYTPRWRLFLEALVDSVAQGIPFQQHQFDKNVFQLEQAFVLSKQRYPSQPRGDTVDLAKKIFLKYYPRWVAGSWGAPGGGSPAEAAAKEAAAKEAAAKEAAAKEAAAKAPSGGGGAPLCGGELVDTLQFVCGDRGFYFSRPASRVSARSRGIVEECCFRSCDLALLETYCATPAKSE

SEQ ID NO: 568 - protein, 828 aa, synthetic construct; identical to SEQ ID NO: 564
  REGION 1..828: note = Synthetic peptide

SEQ ID NO: 569 - protein, 828 aa, synthetic construct
  REGION 1..828: note = Synthetic peptide
MEAVAVAAAVGVLLLAGAGGAAGDEAREAAAVRALVARLLGPGPAADFSVSVERALAAKPGLDTYSLGGGGAARVRVRGSTGVAAAAGLHRYLRDFCGCHVAWSGSQLRLPRPLPAVPGELTEATPNRYRYYQNVCTQSYSFVWWDWARWEREIDWMALNGINLALAWSGQEAIWQRVYLALGLTQAEINEFFTGPAFLAWGRMGNLHTWDGPLPPSWHIKQLYLQHRVLDQMRSFGMTPVLPAFAGHVPEAVTRVFPQVNVTKMGSWGHFNCSYSCSFLLAPEDPIFPIIGSLFLRELIKEFGTDHIYGADTFNEMQPPSSEPSYLAAATTAVYEAMTAVDTEAVWLLQGWLFQHQPQFWGPAQIRAVLGAVPRGRLLVLDLFAESQPVYTRTASFQGQPFIWCMLHNFGGNHGLFGALEAVNGGPEAARLFPNSTMVGTGMAPEGISQNEVVYSLMAELGWRKDPVPDLAAWVTSFAARRYGVSHPDAGAAWRLLLRSVYNCSGEACRGHNRSPLVRRPSLQMNTSIWYNRSDVFEAWRLLLTSAPSLATSPAFRYDLLDLTRQAVQELVSLYYEEARSAYLSKELASLLRAGGVLAYELLPALDEVLASDSRFLLGSWLEQARAAAVSEAEADFYEQNSRYQLTLWGPEGNILDYANKQLAGLVANYYTPRWRLFLEALVDSVAQGIPFQQHQFDKNVFQLEQAFVLSKQRYPSQPRGDTVDLAKKIFLKYYPRWVAGSWGGGGAGGGGAGGGGAGGGGAGGGPSLCGGELVDTLQFVCGDRGFYFSRPASRVSARSRGIVEECCFRSCDLALLETYCATPAKSE

SEQ ID NO: 570 - skipped

SEQ ID NO: 571 - protein, 60 aa, Homo sapiens
LCGGELVDTLQFVCGDRGFYFSRPASRVSRRSRGIVEECCFRSCDLALLETYCATPAKSE

(19) United States
(12) Patent Application Publication
(10) Pub. No.: US 2025/0388644 A1
(43) Pub. Date: Dec. 25, 2025

(54) TARGETED THERAPEUTIC LYSOSOMAL ENZYME FUSION PROTEINS AND USES THEREOF

(71) Applicant: BioMarin Pharmaceutical Inc., Novato, CA (US)

(72) Inventors: Mika Aoyagi-Scharber, Novato, CA (US); Teresa Margaret Christianson, Novato, CA (US); Melita Dvorak-Ewell, Berkeley, CA (US); Daniel J. Wendt, Novato, CA (US); Shinong Long, Novato, CA (US); Jonathan LeBowitz, Novato, CA (US); Daniel Solomon Gold, Novato, CA (US)

(21) Appl. No.: 19/030,525

(22) Filed: Jan. 17, 2025

Related U.S. Application Data
(63) Continuation of application No. 18/759,454, filed on Jun. 28, 2024, now abandoned.
Application No. 18/759,454 is a continuation of application No. 18/503,593, filed on Nov. 7, 2023, now abandoned.
Application No. 18/503,593 is a continuation of application No. 17/572,018, filed on Jan. 10, 2022, now abandoned.
Application No. 17/572,018 is a continuation of application No. 16/378,163, filed on Apr. 8, 2019, now Pat. No. 11,254,725.
Application No. 16/378,163 is a continuation of application No. 15/688,438, filed on Aug. 28, 2017, now Pat. No. 10,301,369.
Application No. 15/688,438 is a division of application No. 14/883,211, filed on Oct. 14, 2015, now Pat. No. 9,771,408.
Application No. 14/883,211 is a division of application No. 14/092,336, filed on Nov. 27, 2013, now Pat. No. 9,376,480.
(60) Provisional application No. 61/730,378, filed on Nov. 27, 2012.
(60) Provisional application No. 61/788,968, filed on Mar. 15, 2013.

(51) Int. Cl. C07K 14/65; A61K 38/00; A61K 38/47; C12N 9/24

(52) U.S. Cl. CPC C07K 14/65; C12N 9/2402; C12Y 302/0105; A61K 38/00; A61K 38/47; C07K 2319/06

(57) ABSTRACT

The present invention relates in general to therapeutic fusion proteins useful to treat lysosomal storage diseases and methods for treating such diseases. Exemplary therapeutic fusion proteins comprise a lysosomal enzyme, a lysosomal targeting moiety, e.g., an IGF-II peptide, and a spacer peptide. Also provided are compositions and methods for treating Mucopolysaccharidosis Type IIIB (Sanfilippo B Syndrome), comprising a targeted therapeutic fusion protein comprising alpha-N-acetylglucosaminidase (Naglu), a lysosomal targeting moiety, e.g., an IGF-II peptide, and a spacer peptide.

### CROSS REFERENCE

This application is a Continuation of U.S. patent application Ser. No. 18/759,454, filed Jun. 28, 2024, which is a Continuation of U.S. patent application Ser. No. 18/503,593, filed Nov. 7, 2023, now abandoned, which is a Continuation of U.S. patent application Ser. No. 17/572,018, filed Jan. 10, 2022, now abandoned, which is a Continuation of U.S. patent application Ser. No. 16/378,163, filed Apr. 8, 2019, now U.S. Pat. No. 11,254,725, issued on Feb. 22, 2022, which is a Continuation of U.S. patent application Ser. No. 15/688,438, filed Aug. 28, 2017, now U.S. Pat. No. 10,301,369, issued on May 28, 2019, which is a Divisional of U.S. patent application Ser. No. 14/883,211, filed Oct. 14, 2015, now U.S. Pat. No. 9,771,408, issued on Sep. 26, 2017, which is a Divisional of U.S. patent application Ser. No. 14/092,336, filed Nov. 27, 2013, now U.S. Pat. No. 9,376,480, issued on Jun. 28, 2016, which claims the priority benefit of U.S. Provisional Application No. 61/1730,378, filed Nov. 27, 2012, and U.S. Provisional Application No. 61/1788,968, filed Mar. 15, 2013, herein incorporated by reference in their entireties.

### SEQUENCE LISTING

The instant application contains a Sequence Listing which has been submitted electronically in XML format and is hereby incorporated by reference in its entirety. Said XML copy, created on Sep. 10, 2025, is named 50535-735_305SL.xml and is 728,158 bytes in size.

### FIELD OF THE INVENTION

The present invention relates in general to therapeutic fusion proteins useful to treat lyssomal storage diseases and methods for treating such diseases. Exemplary therapeutic fusion proteins comprise a lysosomal enzyme, a lysosomal targeting moiety, e.g., an IGF-II peptide, and a spacer peptide. It is contemplated that the lysosomal enzyme is alpha-N-acetylglucosaminidase (Naglu) and the disease is Mucopolysaccharidosis Type IIIB (Sanfilippo B Syndrome).

### BACKGROUND

Normally, mammalian lysosomal enzymes are synthesized in the cytosol and traverse the ER where they are glycosylated with N-linked, high mannose type carbohydrate. In the golgi, the high mannose carbohydrate is modified on lysosomal enzymes by the addition of mannose-6-phosphate (M6P) which targets these proteins to the lysosome. The M6P-modified proteins are delivered to the lysosome via interaction with either of two M6P receptors. The most favorable form of modification is when two M6Ps are added to a high mannose carbohydrate.

More than forty lysosomal storage diseases (LSDs) are caused, directly or indirectly, by the absence of one or more lysosomal enzymes in the lysosome. Enzyme replacement therapy for LSDs is being actively pursued. Therapy generally requires that LSD proteins be taken up and delivered to the lysosomes of a variety of cell types in an M6P-dependent fashion. One possible approach involves purifying an LSD protein and modifying it to incorporate a carbohydrate moiety with M6P. This modified material may be taken up by the cells more efficiently than unmodified LSD proteins due to interaction with M6P receptors on the cell surface.

The inventors of the present application have previously developed a peptide based targeting technology that allows more efficient delivery of therapeutic enzymes to the lysosomes. This proprietary technology is termed Glycosylation Independent Lysosomal Targeting (GILT) because a peptide tag replaces M6P as the moiety targeting the lysosomes. Details of the GILT technology are described in U.S. Application Publication Nos. 2003-0082176, 2004-0006008, 2003-0072761, 2005-0281805, 2005-0244400, and international publications WO 03/032913, WO 03/032727, WO 02/087510, WO 03/102583, WO 2005/078077, the disclosures of all of which are hereby incorporated by reference.

### SUMMARY OF THE INVENTION

The present invention provides further improved compositions and methods for efficient lysosomal targeting based on the GILT technology. Among other things, the present invention provides methods and compositions for targeting lysosomal enzymes to lysosomes using lysosomal targeting peptides. The present invention also provides methods and compositions for targeting lysosomal enzymes to lysosomes using a lysosomal targeting peptide that has reduced or diminished binding affinity for the IGF-1 receptor and/or reduced or diminished binding affinity for the insulin receptor, and/or is resistant to furin cleavage. The present invention also provides lysosomal enzyme fusion proteins comprising a lysosomal enzyme and IGF-II and spacer peptides that provide for improved production and uptake into lysosomes of the lysosomal enzyme fusion protein. In certain embodiments, the lysosomal enzyme is alpha-N-acetylglucosaminidase (Naglu).

In one aspect, the invention provides a targeted therapeutic fusion protein comprising a lysosomal enzyme, a peptide tag having an amino acid sequence at least 70% identical to amino acids 8-67 of mature human IGF-11 and a spacer peptide between the lysosomal enzyme and the IGF-II peptide tag. In various embodiments, the spacer peptide comprises one or more GGGPS (SEQ ID NO: 14) or GGGSP (SEQ ID NO: 15) amino acid sequences, and optionally further comprises one or more of (i) GAP (SEQ ID NO: 9), (ii) GGGGS (SEQ ID NO: 12), (iii) GGGS (SEQ ID NO: 16), (iv) AAAAS (SEQ ID NO: 17), (v) AAAS (SEQ ID NO: 18), (vi) PAPA (SEQ ID NO: 19), (vii) TPAPA (SEQ ID NO: 20), (viii) AAAKE (SEQ ID NO: 21) or (ix) GGGGA (SEQ ID NO: 60).

Exemplary lysosomal enzymes contemplated herein include those set out in Table 1.

In various embodiments, the targeted therapeutic fusion protein comprises an amino acid sequence at least 85% identical to a human a-N-acetylglucosaminidase (Naglu) protein (FIG. 1, SEQ ID NO: 1), a peptide tag having an amino acid sequence at least 70% identical to amino acids 8-67 of mature human IGF-II and a spacer peptide located between the Naglu amino acid sequence and the IGF-II peptide tag. In various embodiments, the spacer comprises the amino acid sequence GAP (SEQ ID NO: 9), GPS (SEQ ID NO: 10), or GGS (SEQ ID NO: 11). In various embodiments, the spacer sequence comprises amino acids Gly-Pro-Ser (GPS) (SEQ ID NO: 10) between the amino acids of mature human IGF-II and the amino acids of human Naglu.

In various embodiments, the spacer peptide comprises one or more GGGGS (SEQ ID NO: 12) or GGGS (SEQ ID NO: 16) amino acid sequences. In various embodiments, the spacer peptide comprises one or more GGGPS (SEQ ID NO: 14) or GGGSP (SEQ ID NO: 15) amino acid sequences. In various embodiments, the spacer peptide comprises one or more AAAAS (SEQ ID NO: 17) or AAAS (SEQ ID NO: 18) amino acid sequences. In various embodiments, the spacer peptide comprises one or more PAPA (SEQ ID NO: 19) or TPAPA (SEQ ID NO: 20) amino acid sequences. In various embodiments, the spacer peptide comprises one or more AAAKE (SEQ ID NO: 21) amino acid sequences. In various embodiments, the spacer peptide comprises one or more GGGGA (SEQ ID NO: 60) amino acid sequences.

In various embodiments, the spacer peptide comprises an amino acid sequence selected from the group consisting of: (GGGGS)n (SEQ ID NOs: 12, 56, 58, 91-94), (GGGGS)n-GGGPS (SEQ ID NOs: 36, 95-100), GAP-(GGGGS)n-GGGPS (SEQ ID NOs: 101-107), GAP-(GGGGS)n-GGGPS-GAP (SEQ ID NOs: 37, 108-113), GAP-(GGGGS)n-GGGPS-(GGGGS)n-GAP (SEQ ID NOs: 114-162), GAP-GGGPS-(GGGGS)n-GAP (SEQ ID NOs: 163-169), GAP-(GGGGS)n-AAAAS-GGGPS-(GGGGS)n-AAAA-GAP (SEQ ID NOs: 170-218), GAP-(GGGGS)n-PAPAP-(Xaa)n-GAP (SEQ ID NOs: 219-267), GAP-(GGGGS)n-PAPAPT-(Xaa)u-GAP (SEQ ID NOs: 268-316), GAP-(GGGGS)n-(Xaa)n-PAPAP-(Xaa)n-(AAAKE)n-(Xaa)n-(GGGGS)n-GAP (SEQ ID NOs: 544-551), (GGGGA)n (SEQ ID NOs: 60, 79, 81, 317-320), (GGGGA)n-GGGPS (SEQ ID NOs: 321-326), GAP-(GGGGA)n-GGGPS (SEQ ID NOs: 327-333), GAP-(GGGGA)n-GGGPS-GAP (SEQ ID NOs: 334-340), GAP-(GGGGA)n-GGGPS-(GGGGA)n-GAP (SEQ ID NOs: 341-389), GAP-GGGPS-(GGGGA)u-GAP (SEQ ID NOs: 390-396), GAP-(GGGGA)u-AAAAS-GGGPS-(GGGGA)u-AAAA-GAP (SEQ ID NOs: 397-445), GAP-(GGGGA)u-PAPAP-(Xaa)u-GAP (SEQ ID NOs: 446-494), GAP-(GGGGA)u-PAPAPT-(Xaa)u-GAP (SEQ ID NOs: 495-543), GAP-(GGGGA)u-(Xaa)n-PAPAP-(Xaa)u-(AAAKE)n-(Xaa)u-(GGGGA)u-GAP (SEQ ID NOs: 552-559); wherein n is 1 to 7. In various embodiments, n is 1 to 4.

In various embodiments, the present invention provides an IGF-II peptide for use as a peptide tag for targeting the peptide or fusion protein comprising the peptide to a mammalian lysosome. In various embodiments, the present invention provides an IGF-II mutein. In various embodiments, the invention provides a furin-resistant IGF-II mutein having an amino acid sequence at least 70% identical to mature human IGF-II (AYRPSETLCGGELVDTLQFVCGDRGFYFSRPASRVSRRSRGIVEECCFRSCDLALLET YCATPAKSE) (SEQ ID NO: 5) and a mutation that abolishes at least one furin protease cleavage site.

In some embodiments, the present invention provides an IGF-II mutein comprising an amino acid sequence at least 70% identical to mature human IGF-II. In various embodiments, the IGF-II mutein peptide tag comprises amino acids 8-67 of mature human IGF-II. In various embodiments, the IGF-II mutein comprises a mutation that reduces or diminishes the binding affinity for the insulin receptor as compared to the wild-type human IGF-II.

In some embodiments, the IGF-II mutein has diminished binding affinity for the IGF-I receptor relative to the affinity of naturally-occurring human IGF-II for the IGF-I receptor.

In various embodiments, the present invention provides a targeted therapeutic fusion protein containing a lysosomal enzyme; and an IGF-II mutein having an amino acid sequence at least 70% identical to mature human IGF-II, wherein the IGF-II mutein is resistant to furin cleavage and binds to the human cation-independent mannose-6-phosphate receptor in a mannose-6-phosphate-independent manner.

In some embodiments, the present invention provides a targeted therapeutic fusion protein containing a lysosomal enzyme; and an IGF-II mutein having an amino acid sequence at least 70% identical to mature human IGF-II, and having diminished binding affinity for the insulin receptor relative to the affinity of naturally-occurring human IGF-II for the insulin receptor. In a related embodiment, the IGF-II mutein is resistant to furin cleavage and binds to the human cation-independent mannose-6-phosphate receptor in a mannose-6-phosphate-independent manner.

In various embodiments, an IGF-II mutein suitable for the invention includes a mutation within a region corresponding to amino acids 30-40 of mature human IGF-II. In some embodiments, an IGF-II mutein suitable for the invention includes a mutation within a region corresponding to amino acids 34-40 of mature human IGF-II such that the mutation abolishes at least one furin protease cleavage site. In some embodiments, a suitable mutation is an amino acid substitution, deletion and/or insertion. In some embodiments, the mutation is an amino acid substitution at a position corresponding to Arg37 or Arg40 of mature human IGF-II. In some embodiments, the amino acid substitution is a Lys or Ala substitution.

In some embodiments, a suitable mutation is a deletion or replacement of amino acid residues corresponding to positions selected from the group consisting of 30-40, 31-40, 32-40, 33-40, 34-40, 30-39, 31-39, 32-39, 34-37, 33-39, 34-39, 35-39, 36-39, 37-40 of mature human IGF-II, and combinations thereof.

In various embodiments, an IGF-II mutein according to the invention further contains a deletion or a replacement of amino acids corresponding to positions 2-7 of mature human IGF-II. In various embodiments, an IGF-II mutein according to the invention further includes a deletion or a replacement of amino acids corresponding to positions 1-7 of mature human IGF-II. In various embodiments, an IGF-II mutein according to the invention further contains a deletion or a replacement of amino acids corresponding to positions 62-67 of mature human IGF-II. In various embodiments, an IGF-II mutein according to the invention further contains an amino acid substitution at a position corresponding to Tyr27, Leu43, or Ser26 of mature human IGF-II. In various embodiments, an IGF-II mutein according to the invention contains at least an amino acid substitution selected from the group consisting of Tyr27Leu, Leu43Val, Ser26Phe and combinations thereof. In various embodiments, an IGF-II mutein according to the invention contains amino acids corresponding to positions 48-55 of mature human IGF-II. In various embodiments, an IGF-II mutein according to the invention contains at least three amino acids selected from the group consisting of amino acids corresponding to positions 8, 48, 49, 50, 54, and 55 of mature human IGF-II. In various embodiments, an IGF-II mutein of the invention contains, at positions corresponding to positions 54 and 55 of mature human IGF-II, amino acids each of which is uncharged or negatively charged at pH 7.4. In various embodiments, the IGF-II mutein has diminished binding affinity for the IGF-I receptor relative to the affinity of naturally-occurring human IGF-II for the IGF-I receptor. In various embodiments, the IGF-II mutein is IGF2 8-67 R37A (i.e., amino acids 8-67 of mature human IGF-II with the Arg at position 37 of mature human IGF-II substituted by Ala).

In various embodiments, the peptide tag is attached to the N-terminus or C-terminus of the lysosomal enzyme, therefore is an N-terminal tag or a C-terminal tag, respectively. In various embodiments, the peptide tag is a C-terminal tag.

In some embodiments, a lysosomal enzyme suitable for the invention is human alpha-N-acetylglucosaminidase (Naglu) (FIG. 1), or a functional fragment or variant thereof. In some embodiments, a lysosomal enzyme suitable for the invention includes amino acids 1-743 of human alpha-N-acetylglucosaminidase or amino acids 24-743 of human alpha-N-acetylglucosaminidase, which lacks a signal sequence.

In various embodiments, a targeted therapeutic fusion protein of the invention further includes a spacer between the lysosomal enzyme and the IGF-11 mutein.

In various embodiments, the spacer comprises an alpha-helical structure or a rigid structure.

In various embodiments, the spacer comprises one or more Gly-Ala-Pro (GAP) (SEQ ID NO: 9), Gly-Pro-Ser (GPS) (SEQ ID NO: 10), or Gly-Gly-Ser (GGS) (SEQ ID NO: 11) amino acid sequences.

In some embodiments, the spacer is selected from the group consisting of

(SEQ ID NO: 22)
EFGGGGSTR,
(SEQ ID NO: 9)
GAP,
(SEQ ID NO: 12)
GGGGS,
(SEQ ID NO: 23)
GPSGSPG,
(SEQ ID NO: 24)
GPSGSPGT,
(SEQ ID NO: 25)
GPSGSPGH,
(SEQ ID NO: 26)
GGGGSGGGGSGGGGSGGGGSGGGPST,
(SEQ ID NO: 27)
GGGGSGGGGSGGGGSGGGGSGGGPSH,
(SEQ ID NO: 28)
GGGGSGGGGSGGGGSGGGGSGGGPSGGGGSGGGPS,
(SEQ ID NO: 29)
GAPGGGGSGGGGSGGGGSGGGGSGGGPSGGGGSGGGPSGAP,
(SEQ ID NO: 30)
GGGGSGGGGSGGGGSGGGGSGGGPSGGGGSGGGGSGGGGSG
GGGSGGGPS,
(SEQ ID NO: 31)
GAPGGGGSGGGGSGGGGSGGGGSGGGPSGGGGSGGGGSGGGG
SGGGGSGGGPSGAP,
(SEQ ID NO: 32)
GGGGSGGGGSGGGGSGGGPSGGGGSGGGGSGGGPS,
(SEQ ID NO: 33)
GAPGGGGSGGGGSGGGGSGGGPSGGGGSGGGGSGGGPSGAP,
(SEQ ID NO: 34)
GGGGSGGGGSGGGGSGGGPSGGGGSGGGGSGGGPSGGGGSGG
GGSGGGPS,
(SEQ ID NO: 35)
GAPGGGGSGGGGSGGGGSGGGPSGGGGSGGGGSGGGPSGGGGS
GGGGSGGGPSGAP,
(SEQ ID NO: 36)
GGGGSGGGGSGGGGSGGGGSGGGPS,
(SEQ ID NO: 37)
GAPGGGGSGGGGSGGGGSGGGGSGGGPSGAP,
(SEQ ID NO: 38)
GGGGSGGGGSAAAASGGGGSGGGPS,
(SEQ ID NO: 39)
GAPGGGGSGGGGSAAAASGGGGSGGGPSGAP,
(SEQ ID NO: 40)
GGGGSGGGGSAAAASGGGGSGGGGSAAAASGGGGSG
GGGSAAAASGGGPS,
(SEQ ID NO: 41)
GAPGGGGSGGGGSAAAASGGGGSGGGGSAAAASGGGGS
GGGGSAAAASGGGPSGAP,
(SEQ ID NO: 42)
GGGGSGGGGSAAAASGGGPSGGGGSAAAASGGGPSGGG
GSAAAASGGGPS,
(SEQ ID NO: 43)
GAPGGGGSGGGGSAAAASGGGPSGGGGSAAAASGGGPS
GGGGSAAAASGGGPSGAP,
(SEQ ID NO: 44)
GGSPAGSPTSTEEGTSESATPESGPGTSTEPSEGSAPG
SPAGSPGPS,
(SEQ ID NO: 45)
GAPGGSPAGSPTSTEEGTSESATPESGPGTSTEPSEGSA
PGSPAGSPGPSGAP,
(SEQ ID NO: 46)
GGSPAGSPTSTEEGTSESATPESGPGTSTEPSEGSAPGS
PAGSPTSTGPS,
(SEQ ID NO: 47)
GAPGGSPAGSPTSTEEGTSESATPESGPGTSTEPSEGSA
PGSPAGSPTSTGPSGAP,
(SEQ ID NO: 48)
GGGSPAPTPTPAPTPAPTPAGGGPS,
(SEQ ID NO: 49)
GAPGGGSPAPTPTPAPTPAPTPAGGGPSGAP,
(SEQ ID NO: 50)
GGGSPAPAPTPAPAPTPAPAGGGPS,
(SEQ ID NO: 51)
GAPGGGSPAPAPTPAPAPTPAPAGGGPSGAP,
(SEQ ID NO: 52)
GGGSAEAAAKEAAAKEAAAKAGGPS,
(SEQ ID NO: 53)
GAPGGGSAEAAAKEAAAKEAAAKAGGPSGAP,
(SEQ ID NO: 54)
GGGSPAEAAAKEAAAKEAAAKEAAAKEAAAKAPSGGG,
(SEQ ID NO: 55)
GAPGGGSPAEAAAKEAAAKEAAAKEAAAKEAAAKAPSGGGGAP,
(SEQ ID NO: 56)
GGGGSGGGGSGGGGS,
(SEQ ID NO: 57)
GAPGGGGSGGGGSGGGGSGAP,
(SEQ ID NO: 58)
GGGGSGGGGSGGGGSGGGGS,
(SEQ ID NO: 59)
GAPGGGGSGGGGSGGGGSGGGGSGAP,
(SEQ ID NO: 60)
GGGGA,
(SEQ ID NO: 61)
GGGGAGGGGAGGGGAGGGGAGGGPST,
(SEQ ID NO: 62)
GGGGAGGGGAGGGGAGGGGAGGGPSH,
(SEQ ID NO: 63)
GGGGAGGGGAGGGGAGGGGAGGGPSGGGGAGGGPS,
(SEQ ID NO: 64)
GAPGGGGAGGGGAGGGGAGGGGAGGGPSGGGGAGGGPSGAP,
(SEQ ID NO: 65)
GGGGAGGGGAGGGGAGGGGAGGGPSGGGGAGGGGAGGGGAGG
GGAGGGPS,
(SEQ ID NO: 66)
GAPGGGGAGGGGAGGGGAGGGGAGGGPSGGGGAGGGGAGGGG
AGGGGAGGGPSGAP,
(SEQ ID NO: 67)
GGGGAGGGGAGGGGAGGGPSGGGGAGGGGAGGGPS,
(SEQ ID NO: 68)
GAPGGGGAGGGGAGGGGAGGGPSGGGGAGGGGAGGGPSGAP,
(SEQ ID NO: 69)
GGGGAGGGGAGGGGAGGGPSGGGGAGGGGAGGGPSGGGGAGG
GGAGGGPS,
(SEQ ID NO: 70)
GAPGGGGAGGGGAGGGGAGGGPSGGGGAGGGGAGGGPSGGGG
AGGGGAGGGPSGAP,
(SEQ ID NO: 71)
GGGGAGGGGAGGGGAGGGGAGGGPS,
(SEQ ID NO: 72)
GAPGGGGAGGGGAGGGGAGGGGAGGGPSGAP,
(SEQ ID NO: 73)
GGGGAGGGGAAAAASGGGGAGGGPS,
(SEQ ID NO: 74)
GAPGGGGAGGGGAAAAASGGGGAGGGPSGAP,
(SEQ ID NO: 75)
GGGGAGGGGAAAAASGGGGAGGGGAAAAASGGGGAGGGGAAA
AASGGGPS,
(SEQ ID NO: 76)
GAPGGGGAGGGGAAAAASGGGGAGGGGAAAAASGGGGAGGGGA
AAAASGGGPSGAP,
(SEQ ID NO: 77)
GGGGAGGGGAAAAASGGGPSGGGGAAAAASGGGPSGGGGAAAA
ASGGGPS,
(SEQ ID NO: 78)
GAPGGGGAGGGGAAAAASGGGPSGGGGAAAAASGGGPSGGGGAA
AAASGGGPSGAP,
(SEQ ID NO: 79)
GGGGAGGGGAGGGGA,
(SEQ ID NO: 80)
GAPGGGGAGGGGAGGGGAGAP,
(SEQ ID NO: 81)
GGGGAGGGGAGGGGAGGGGA,
(SEQ ID NO: 82)
GAPGGGGAGGGGAGGGGAGGGGAGAP,
GGGGAGGGGAGGGGAGGGGAGGGGAGGGGAGGGGAGGGGAGG
GPS,
[or
(SEQ ID NO: 83)
(GGGGA)₈GGGPS],
GGGGAGGGGAGGGGAGGGGAGGGGAGGGGAGGGGAGGGGAGGG
PSH
[or
(SEQ ID NO: 84)
(GGGGA)₈GGGPSH],
GGGGAGGGGAGGGGAGGGGAGGGGAGGGGAGGGGAGGGGAGGGGA
GGGPS
[or
(SEQ ID NO: 85)
(GGGGA)₉GGGPS],
GGGGAGGGGAGGGGAGGGGAGGGGAGGGGAGGGGAGGGGAGGGG
AGGGPSH
[or
(SEQ ID NO: 86)
(GGGGA)₉GGGPSH],
(SEQ ID NO: 87)
GGGGPAPGPGPAPGPAPGPAGGGPS,
(SEQ ID NO: 88)
GAPGGGGPAPGPGPAPGPAPGPAGGGPGGAP,
(SEQ ID NO: 89)
GGGGPAPAPGPAPAPGPAPAGGGPS,
and
(SEQ ID NO: 90)
GAPGGGGPAPAPGPAPAPGPAPAGGGPGGAP.

In some embodiments, the spacer is selected from the group consisting of

(SEQ ID NO: 36)
GGGGSGGGGSGGGGSGGGGSGGGPS,
(SEQ ID NO: 44)
GGSPAGSPTSTEEGTSESATPESGPGTSTEPSEGSAPGSPAGSP
GPS,
(SEQ ID NO: 45)
GAPGGSPAGSPTSTEEGTSESATPESGPGTSTEPSEGSAPGSPA
GSPGPSGAP,
(SEQ ID NO: 46)
GGSPAGSPTSTEEGTSESATPESGPGTSTEPSEGSAPGSPAGSP
TSTGPS,
(SEQ ID NO: 47)
GAPGGSPAGSPTSTEEGTSESATPESGPGTSTEPSEGSAPGSPA
GSPTSTGPSGAP,
(SEQ ID NO: 48)
GGGSPAPTPTPAPTPAPTPAGGGPS,
(SEQ ID NO: 49)
GAPGGGSPAPTPTPAPTPAPTPAGGGPSGAP,
(SEQ ID NO: 50)
GGGSPAPAPTPAPAPTPAPAGGGPS,
(SEQ ID NO: 51)
GAPGGGSPAPAPTPAPAPTPAPAGGGPSGAP,
(SEQ ID NO: 52)
GGGSAEAAAKEAAAKEAAAKAGGPS,
(SEQ ID NO: 53)
GAPGGGSAEAAAKEAAAKEAAAKAGGPSGAP,
(SEQ ID NO: 54)
GGGSPAEAAAKEAAAKEAAAKEAAAKEAAAKAPSGGG,
(SEQ ID NO: 55)
GAPGGGSPAEAAAKEAAAKEAAAKEAAAKEAAAKAPSGGGGAP,
and
(SEQ ID NO: 71)
GGGGAGGGGAGGGGAGGGGAGGGPS.

In some embodiments, the spacer is selected from the group consisting of

(SEQ ID NO: 36)
GGGGSGGGGSGGGGSGGGGSGGGPS,
(SEQ ID NO: 47)
GAPGGSPAGSPTSTEEGTSESATPESGPGTSTEPS,
EGSAPGSPAGSPTSTGPSGAP,
(SEQ ID NO: 51)
GAPGGGSPAPAPTPAPAPTPAPAGGGPSGAP,
(SEQ ID NO: 55)
GAPGGGSPAEAAAKEAAAKEAAAKEAAAKEAAAKAPSGGGGAP,
and
(SEQ ID NO: 71)
GGGGAGGGGAGGGGAGGGGAGGGPS.

In various embodiments, the fusion protein further comprises a pharmaceutically acceptable carrier, diluents or excipient.

The present invention also provides nucleic acids encoding the IGF-II mutein or the targeted therapeutic fusion protein as described in various embodiments above. The present invention further provides various cells containing the nucleic acid of the invention.

The present invention provides pharmaceutical compositions suitable for treating lysosomal storage disease containing a therapeutically effective amount of a targeted therapeutic fusion protein of the invention. The invention further provides methods of treating lysosomal storage diseases comprising administering to a subject in need of treatment a targeted therapeutic fusion protein according to the invention. In some embodiments, the lysosomal storage disease is Mucopolysaccharidosis Type IIIB (Sanfilippo B Syndrome).

In another aspect, the present invention provides a method of producing a targeted therapeutic fusion protein including a step of culturing mammalian cells in a cell culture medium, wherein the mammalian cells carry the nucleic acid of the invention, in particular, as described in various embodiments herein; and the culturing is performed under conditions that permit expression of the targeted therapeutic fusion protein.

In yet another aspect, the present invention provides a method of producing a targeted therapeutic fusion protein including a step of culturing furin-deficient cells (e.g., furin-deficient mammalian cells) in a cell culture medium, wherein the furin-deficient cells carry a nucleic acid encoding a fusion protein comprising a lysosomal enzyme and an IGF-II mutein having an amino acid sequence at least 70% identical to mature human IGF-II, wherein the IGF-II mutein binds to the human cation-independent mannose-6-phosphate receptor in a mannose-6-phosphate-independent manner; and wherein the culturing is performed under conditions that permit expression of the targeted therapeutic fusion protein.

In various embodiments, it is contemplated that certain of the targeted therapeutic proteins comprising a spacer as described herein exhibit increased expression of active protein when expressed recombinantly compared to targeted therapeutic proteins comprising a different spacer peptide. In various embodiments, it is also contemplated that targeted therapeutic proteins described herein may have increased activity compared to other targeted therapeutic proteins herein. It is contemplated that those targeted therapeutic proteins exhibiting increased expression of active protein and/or having increased activity compared to other targeted therapeutic proteins comprising a different spacer peptide are used for further experimentation.

In another aspect, the invention provides a method for treating a lysosomal storage disease in a subject comprising administering to the subject a therapeutically effective amount of a pharmaceutical composition comprising a fusion protein comprising a lysosomal enzyme, a peptide tag having an amino acid sequence at least 70% identical to amino acids 8-67 of mature human IGF-II and a spacer peptide located between the lysosomal enzyme amino acid sequence and the IGF-II peptide tag. In various embodiments, the spacer peptide comprises one or more GGGPS (SEQ ID NO: 14) or GGGSP (SEQ ID NO: 15) amino acid sequences, and optionally further comprises one or more of (i) GAP (SEQ ID NO: 9), (ii) GGGGS (SEQ ID NO: 12), (iii) GGGS (SEQ ID NO: 16), (iv) AAAAS (SEQ ID NO: 17), (v) AAAS (SEQ ID NO: 18), (vi) PAPA (SEQ ID NO: 19), (vii) TPAPA (SEQ ID NO: 20), (viii) AAAKE (SEQ ID NO: 21) or (ix) GGGGA (SEQ ID NO: 60).

In various embodiments, the spacer peptide comprises an amino acid sequence selected from the group consisting of: (GGGGS)n (SEQ ID NOs: 12, 56, 58, 91-94), (GGGGS)n-GGGPS (SEQ ID NOs: 36, 95-100), GAP-(GGGGS)n-GGGPS (SEQ ID NOs: 101-107), GAP-(GGGGS)n-GGGPS-GAP (SEQ ID NOs: 37, 108-113), GAP-(GGGGS)n-GGGPS-(GGGGS)n-GAP (SEQ ID NOs: 114-162), GAP-GGGPS-(GGGGS)n-GAP (SEQ ID NOs: 163-169), GAP-(GGGGS)n-AAAAS-GGGPS-(GGGGS)n-AAAA-GAP (SEQ ID NOs: 170-218), GAP-(GGGGS)n-PAPAP-(Xaa)n-GAP (SEQ ID NOs: 219-267), GAP-(GGGGS)n-PAPAPT-(Xaa)n-GAP (SEQ ID NOs: 268-316), GAP-(GGGGS)n-(Xaa)n-PAPAP-(Xaa)n-(AAAKE)n-(Xaa)n-(GGGGS)n-GAP (SEQ ID NOs: 544-551), (GGGGA)n (SEQ ID NOs: 60, 79, 81, 317-320), (GGGGA)n-GGGPS (SEQ ID NOs: 321-326), GAP-(GGGGA)n-GGGPS (SEQ ID NOs: 327-333), GAP-(GGGGA)n-GGGPS-GAP (SEQ ID NOs: 334-340), GAP-(GGGGA)n-GGGPS-(GGGGA)n-GAP (SEQ ID NOs: 341-389), GAP-GGGPS-(GGGGA)n-GAP (SEQ ID NOs: 390-396), GAP-(GGGGA)n-AAAAS-GGGPS-(GGGGA)n-AAAA-GAP (SEQ ID NOs: 397-445), GAP-(GGGGA)11-PAPAP-(Xaa)11-GAP (SEQ ID NOs: 446-494), GAP-(GGGGA)11-PAPAPT-(Xaa)n-GAP (SEQ ID NOs: 495-543), GAP-(GGGGA)ll-(Xaa)n-PAPAP-(Xaa)11-(AAAKE)n-(Xaa)11-(GGGGA)11-GAP (SEQ ID NOs: 552-559); wherein n is 1 to 7, optionally n is 1 to 4.

In various embodiments, the spacer peptide has an amino acid sequence selected from the group consisting of EFGGGGSTR (SEQ ID NO: 22), GAP (SEQ ID NO: 9), GGGGS (SEQ ID NO: 12), GPSGSPG (SEQ ID NO: 23), GPSGSPGT (SEQ ID NO: 24), GPSGSPGH (SEQ ID NO: 25), GGGGSGGGGSGGGGSGGGGSGGGPST (SEQ ID NO: 26), GGGGSGGGGSGGGGSGGGGSGGGPSH (SEQ ID NO: 27), GGGGSGGGGSGGGGSGGGGSGGGPSGGGGSGGGPS (SEQ ID NO: 28), GAPGGGGSGGGGSGGGGSGGGGSGGGPSGGGGSGGGPSGAP (SEQ ID NO: 29), GGGGSGGGGSGGGGSGGGGSGGGPSGGGGSGGGGSGGGGSGGGGSGGGPS (SEQ ID NO: 30), GAPGGGGSGGGGSGGGGSGGGGSGGGPSGGGGSGGGGSGGGGSGGGGSGGGPSGA P (SEQ ID NO: 31), GGGGSGGGGSGGGGSGGGPSGGGGSGGGGSGGGPS (SEQ ID NO: 32), GAPGGGGSGGGGSGGGGSGGGPSGGGGSGGGGSGGGPSGAP (SEQ ID NO: 33), GGGGSGGGGSGGGGSGGGPSGGGGSGGGGSGGGPSGGGGSGGGGSGGGPS (SEQ ID NO: 34), GAPGGGGSGGGGSGGGGSGGGPSGGGGSGGGGSGGGPSGGGGSGGGGSGGGPSGA P (SEQ ID NO: 35), GGGGSGGGGSGGGGSGGGGSGGGPS (SEQ ID NO: 36), GAPGGGGSGGGGSGGGGSGGGGSGGGPSGAP (SEQ ID NO: 37), GGGGSGGGGSAAAASGGGGSGGGPS (SEQ ID NO: 38), GAPGGGGSGGGGSAAAASGGGGSGGGPSGAP (SEQ ID NO: 39), GGGGSGGGGSAAAASGGGGSGGGGSAAAASGGGGSGGGGSAAAASGGGPS (SEQ ID NO: 40), GAPGGGGSGGGGSAAAASGGGGSGGGGSAAAASGGGGSGGGGSAAAASGGGPSG AP (SEQ ID NO: 41), GGGGSGGGGSAAAASGGGPSGGGGSAAAASGGGPSGGGGSAAAASGGGPS (SEQ ID NO: 42), GAPGGGGSGGGGSAAAASGGGPSGGGGSAAAASGGGPSGGGGSAAAASGGGPSGA P (SEQ ID NO: 43), GGSPAGSPTSTEEGTSESATPESGPGTSTEPSEGSAPGSPAGSPGPS (SEQ ID NO: 44), GAPGGSPAGSPTSTEEGTSESATPESGPGTSTEPSEGSAPGSPAGSPGPSGAP (SEQID NO: 45), GGSPAGSPTSTEEGTSESATPESGPGTSTEPSEGSAPGSPAGSPTSTGPS (SEQ ID NO: 46), GAPGGSPAGSPTSTEEGTSESATPESGPGTSTEPSEGSAPGSPAGSPTSTGPSGAP (SEQ ID NO: 47), GGGSPAPTPTPAPTPAPTPAGGGPS (SEQ ID NO: 48), GAPGGGSPAPTPTPAPTPAPTPAGGGPSGAP (SEQ ID NO: 49), GGGSPAPAPTPAPAPTPAPAGGGPS (SEQ ID NO: 50), GAPGGGSPAPAPTPAPAPTPAPAGGGPSGAP (SEQ ID NO: 51), GGGSAEAAAKEAAAKEAAAKAGGPS (SEQ ID NO: 52), GAPGGGSAEAAAKEAAAKEAAAKAGGPSGAP (SEQ ID NO: 53), GGGSPAEAAAKEAAAKEAAAKEAAAKEAAAKAPSGGG (SEQ ID NO: 54), GAPGGGSPAEAAAKEAAAKEAAAKEAAAKEAAAKAPSGGGGAP (SEQ ID NO: 55), GGGGSGGGGSGGGGS (SEQ ID NO: 56), GAPGGGGSGGGGSGGGGSGAP (SEQ ID NO: 57), GGGGSGGGGSGGGGSGGGGS (SEQ ID NO: 58), GAPGGGGSGGGGSGGGGSGGGGSGAP (SEQ ID NO: 59), GGGGA (SEQ ID NO: 60), GGGGAGGGGAGGGGAGGGGAGGGPST (SEQ ID NO: 61), GGGGAGGGGAGGGGAGGGGAGGGPSH (SEQ ID NO: 62), GGGGAGGGGAGGGGAGGGGAGGGPSGGGGAGGGPS (SEQ ID NO: 63), GAPGGGGAGGGGAGGGGAGGGGAGGGPSGGGGAGGGPSGAP (SEQ ID NO: 64), GGGGAGGGGAGGGGAGGGGAGGGPSGGGGAGGGGAGGGGAGGGGAGGGPS (SEQ ID NO: 65), GAPGGGGAGGGGAGGGGAGGGGAGGGPSGGGGAGGGGAGGGGAGGGGAGGGPS GAP (SEQ ID N0:66), GGGGAGGGGAGGGGAGGGPSGGGGAGGGGAGGGPS (SEQ ID NO: 67), GAPGGGGAGGGGAGGGGAGGGPSGGGGAGGGGAGGGPSGAP (SEQ ID NO: 68), GGGGAGGGGAGGGGAGGGPSGGGGAGGGGAGGGPSGGGGAGGGGAGGGPS (SEQ ID NO: 69), GAPGGGGAGGGGAGGGGAGGGPSGGGGAGGGGAGGGPSGGGGAGGGGAGGGPS GAP (SEQ ID NO: 70), GGGGAGGGGAGGGGAGGGGAGGGPS (SEQ ID NO: 71), GAPGGGGAGGGGAGGGGAGGGGAGGGPSGAP (SEQ ID NO: 72), GGGGAGGGGAAAAASGGGGAGGGPS (SEQ ID NO: 73), GAPGGGGAGGGGAAAAASGGGGAGGGPSGAP (SEQ ID NO: 74), GGGGAGGGGAAAAASGGGGAGGGGAAAAASGGGGAGGGGAAAAASGGGPS (SEQ ID NO: 75), GAPGGGGAGGGGAAAAASGGGGAGGGGAAAAASGGGGAGGGGAAAAASGGGPS GAP (SEQ ID NO: 76), GGGGAGGGGAAAAASGGGPSGGGGAAAAASGGGPSGGGGAAAAASGGGPS (SEQ ID NO: 77), GAPGGGGAGGGGAAAAASGGGPSGGGGAAAAASGGGPSGGGGAAAAASGGGPSG AP (SEQ ID NO: 78), GGGGAGGGGAGGGGA (SEQ ID NO: 79), GAPGGGGAGGGGAGGGGAGAP (SEQ ID NO: 80), GGGGAGGGGAGGGGAGGGGA (SEQ ID NO: 81), GAPGGGGAGGGGAGGGGAGGGGAGAP (SEQ ID NO: 82), GGGGAGGGGAGGGGAGGGGAGGGGAGGGGAGGGGAGGGGAGGGPS [or (GGGGA)sGGGPS](SEQ ID NO: 83), GGGGAGGGGAGGGGAGGGGAGGGGAGGGGAGGGGAGGGGAGGGPSH [or (GGGGA)sGGGPSH](SEQ ID NO: 84), GGGGAGGGGAGGGGAGGGGAGGGGAGGGGAGGGGAGGGGAGGGGAGGGPS [or (GGGGA)₉GGGPS](SEQ ID NO: 85), GGGGAGGGGAGGGGAGGGGAGGGGAGGGGAGGGGAGGGGAGGGGAGGGPSH [or (GGGGA)₉GGGPSH](SEQ ID NO: 86), GGGGPAPGPGPAPGPAPGPAGGGPS (SEQ ID NO: 87), GAPGGGGPAPGPGPAPGPAPGPAGGGPGGAP (SEQ ID NO: 88), GGGGPAPAPGPAPAPGPAPAGGGPS (SEQ ID NO: 89), and GAPGGGGPAPAPGPAPAPGPAPAGGGPGGAP (SEQ ID NO: 90).

In various embodiments, the spacer peptide has an amino acid sequence selected from the group consisting of GGGGSGGGGSGGGGSGGGGSGGGPS (SEQ ID NO: 36), GGSPAGSPTSTEEGTSESATPESGPGTSTEPSEGSAPGSPAGSPGPS (SEQ ID NO: 44), GAPGGSPAGSPTSTEEGTSESATPESGPGTSTEPSEGSAPGSPAGSPGPSGAP (SEQID NO: 45), GGSPAGSPTSTEEGTSESATPESGPGTSTEPSEGSAPGSPAGSPTSTGPS (SEQ ID NO: 46), GAPGGSPAGSPTSTEEGTSESATPESGPGTSTEPSEGSAPGSPAGSPTSTGPSGAP (SEQ ID NO: 47), GGGSPAPTPTPAPTPAPTPAGGGPS (SEQ ID NO: 48), GAPGGGSPAPTPTPAPTPAPTPAGGGPSGAP (SEQ ID NO: 49), GGGSPAPAPTPAPAPTPAPAGGGPS (SEQ ID NO: 50), GAPGGGSPAPAPTPAPAPTPAPAGGGPSGAP (SEQ ID NO: 51), GGGSAEAAAKEAAAKEAAAKAGGPS (SEQ ID NO: 52), GAPGGGSAEAAAKEAAAKEAAAKAGGPSGAP (SEQ ID NO: 53), GGGSPAEAAAKEAAAKEAAAKEAAAKEAAAKAPSGGG (SEQ ID NO: 54), GAPGGGSPAEAAAKEAAAKEAAAKEAAAKEAAAKAPSGGGGAP (SEQ ID NO: 55), and GGGGAGGGGAGGGGAGGGGAGGGPS (SEQ ID NO: 71).

In various embodiments, the spacer peptide has an amino acid sequence selected from the group consisting of GGGGSGGGGSGGGGSGGGGSGGGPS (SEQ ID NO: 36), GAPGGSPAGSPTSTEEGTSESATPESGPGTSTEPSEGSAPGSPAGSPTSTGPSGAP (SEQ ID NO: 47), GAPGGGSPAPAPTPAPAPTPAPAGGGPSGAP (SEQ ID NO: 51), GAPGGGSPAEAAAKEAAAKEAAAKEAAAKEAAAKAPSGGGGAP (SEQ ID NO: 55), and GGGGAGGGGAGGGGAGGGGAGGGPS (SEQ ID NO: 71).

Exemplary lysosomal storage diseases contemplated by the methods herein include those set out in Table 1. It is contemplated that the lysosomal storage disease is treated using a targeted therapeutic fusion protein comprising the enzyme deficient in the lysosomal storage disease, also disclosed in Table 1.

In various embodiments, the invention provides a method for treating Mucopolysaccharidosis Type IIIB (Sanfilippo B Syndrome) in a subject comprising administering to the subject a therapeutically effective amount of a pharmaceutical composition comprising a fusion protein comprising an amino acid sequence at least 85% identical to a human a-N-acetylglucosaminidase (Naglu) protein (SEQ ID NO: 1), a peptide tag having an amino acid sequence at least 70% identical to amino acids 8-67 of mature human IGF-II and a spacer peptide located between the Naglu amino acid sequence and the IGF-II peptide tag. In various embodiments, the spacer comprises the amino acid sequence GAP (SEQ ID NO: 9), GPS (SEQ ID NO: 10), or GGS (SEQ ID NO: 11).

In various embodiments, the spacer sequence comprises amino acids Gly-Pro-Ser (GPS) (SEQ ID NO: 10) between the amino acids of mature human IGF-II and the amino acids of human Naglu.

In various embodiments, the spacer peptide comprises one or more GGGGS (SEQ ID NO: 12) or GGGS (SEQ ID NO: 16) amino acid sequences. In various embodiments, the spacer peptide comprises one or more GGGPS (SEQ ID NO: 14) or GGGSP (SEQ ID NO: 15) amino acid sequences. In various embodiments, the spacer peptide comprises one or more AAAAS (SEQ ID NO: 17) or AAAS (SEQ ID NO: 18) amino acid sequences. In various embodiments, the spacer peptide comprises one or more PAPA (SEQ ID NO: 19) or TPAPA (SEQ ID NO: 20) amino acid sequences. In various embodiments, the spacer peptide comprises one or more AAAKE (SEQ ID NO: 21) amino acid sequences. In various embodiments, the spacer peptide comprises one or more GGGGA (SEQ ID NO: 60) amino acid sequences.

In various embodiments, the spacer peptide comprises an amino acid sequence selected from the group consisting of: (GGGGS)n (SEQ ID NOs: 12, 56, 58, 91-94), (GGGGS)n-GGGPS (SEQ ID NOs: 36, 95-100), GAP-(GGGGS)n-GGGPS (SEQ ID NOs: 101-107), GAP-(GGGGS)n-GGGPS-GAP (SEQ ID NOs: 37, 108-113), GAP-(GGGGS)n-GGGPS-(GGGGS)n-GAP (SEQ ID NOs: 114-162), GAP-GGGPS-(GGGGS)n-GAP (SEQ ID NOs: 163-169), GAP-(GGGGS)n-AAAAS-GGGPS-(GGGGS)n-AAAA-GAP (SEQ ID NOs: 170-218), GAP-(GGGGS)n-PAPAP-(Xaa)n-GAP (SEQ ID NOs: 219-267), GAP-(GGGGS)n-PAPAPT-(Xaa)n-GAP (SEQ ID NOs: 268-316), GAP-(GGGGS)n-(Xaa)n-PAPAP-(Xaa)n-(AAAKE)n-(Xaa)n-(GGGGS)n-GAP (SEQ ID NOs: 544-551), (GGGGA)n (SEQ ID NOs: 60, 79, 81, 317-320), (GGGGA)n-GGGPS (SEQ ID NOs: 321-326), GAP-(GGGGA)n-GGGPS (SEQ ID NOs: 327-333), GAP-(GGGGA)n-GGGPS-GAP (SEQ ID NOs: 334-340), GAP-(GGGGA)n-GGGPS-(GGGGA)n-GAP (SEQ ID NOs: 341-389), GAP-GGGPS-(GGGGA)n-GAP (SEQ ID NOs: 390-396), GAP-(GGGGA)n-AAAAS-GGGPS-(GGGGA)n-AAAA-GAP (SEQ ID NOs: 397-445), GAP-(GGGGA)n-PAPAP-(Xaa)n-GAP (SEQ ID NOs: 446-494), GAP-(GGGGA)n-PAPAPT-(Xaa)n-GAP (SEQ ID NOs: 495-543).

[symbol]

(GGGGA)u-(Xaa)n-PAPAP-(Xaa)u-(AAAKE)n-(Xaa)u-(GGGGA)u-GAP (SEQ ID NOs: 552-559); wherein n is 1 to 7, optionally wherein n is 1 to 4.

In various embodiments, the invention provides a method for reducing glycosaminoglycan (GAG) levels in vivo comprising administering to a subject suffering from Mucopolysaccharidosis Type IIIB (Sanfilippo B Syndrome) an effective amount of a fusion protein comprising i) an amino acid sequence at least 85% identical to a humana-N-acetylglucosaminidase (Naglu) protein (SEQ ID NO: 1), ii) a peptide tag having an amino acid sequence at least 70% identical to amino acids 8-67 of mature human IGF-II, and iii) a spacer peptide located between the Naglu amino acid sequence and the IGF-II peptide tag.

In various embodiments, the spacer sequence complises one or more copies of amino acids Gly-Ala-Pro (GAP) (SEQ ID NO: 9) between the amino acids of mature human IGF-II and the amino acids of human Naglu.

In various embodiments, the spacer peptide is selected from the group consisting of

(SEQ ID NO: 22)
EFGGGGSTR,
(SEQ ID NO: 9)
GAP,
(SEQ ID NO: 12)
GGGGS,
(SEQ ID NO: 23)
GPSGSPG,
(SEQ ID NO: 24)
GPSGSPGT,
(SEQ ID NO: 25)
GPSGSPGH,
(SEQ ID NO: 26)
GGGGSGGGGSGGGGSGGGGSGGGPST,
(SEQ ID NO: 27)
GGGGSGGGGSGGGGSGGGGSGGGPSH,
(SEQ ID NO: 28)
GGGGSGGGGSGGGGSGGGGSGGGPSGGGGSGGGPS,
(SEQ ID NO: 29)
GAPGGGGSGGGGSGGGGSGGGGSGGGPSGGGGSGGGPSGAP,
(SEQ ID NO: 30)
GGGGSGGGGSGGGGSGGGGSGGGPSGGGGSGGGGSGGGGSG
GGGSGGGPS,
(SEQ ID NO: 31)
GAPGGGGSGGGGSGGGGSGGGGSGGGPSGGGGSGGGGSGGGG
SGGGGSGGGPSGAP,
(SEQ ID NO: 32)
GGGGSGGGGSGGGGSGGGPSGGGGSGGGGSGGGPS,
(SEQ ID NO: 33)
GAPGGGGSGGGGSGGGGSGGGPSGGGGSGGGGSGGGPSGAP,
(SEQ ID NO: 34)
GGGGSGGGGSGGGGSGGGPSGGGGSGGGGSGGGPSGGGGSGG
GGSGGGPS,
(SEQ ID NO: 35)
GAPGGGGSGGGGSGGGGSGGGPSGGGGSGGGGSGGGPSGGGGS
GGGGSGGGPSGAP,
(SEQ ID NO: 36)
GGGGSGGGGSGGGGSGGGGSGGGPS,
(SEQ ID NO: 37)
GAPGGGGSGGGGSGGGGSGGGGSGGGPSGAP,
(SEQ ID NO: 38)
GGGGSGGGGSAAAASGGGGSGGGPS,
(SEQ ID NO: 39)
GAPGGGGSGGGGSAAAASGGGGSGGGPSGAP,
(SEQ ID NO: 40)
GGGGSGGGGSAAAASGGGGSGGGGSAAAASGGGGSG
GGGSAAAASGGGPS,
(SEQ ID NO: 41)
GAPGGGGSGGGGSAAAASGGGGSGGGGSAAAASGGGGS
GGGGSAAAASGGGPSGAP,
(SEQ ID NO: 42)
GGGGSGGGGSAAAASGGGPSGGGGSAAAASGGGPSGGG
GSAAAASGGGPS,
(SEQ ID NO: 43)
GAPGGGGSGGGGSAAAASGGGPSGGGGSAAAASGGGPS
GGGGSAAAASGGGPSGAP,
(SEQ ID NO: 44)
GGSPAGSPTSTEEGTSESATPESGPGTSTEPSEGSAPG
SPAGSPGPS,
(SEQ ID NO: 45)
GAPGGSPAGSPTSTEEGTSESATPESGPGTSTEPSEGSA
PGSPAGSPGPSGAP,
(SEQ ID NO: 46)
GGSPAGSPTSTEEGTSESATPESGPGTSTEPSEGSAPGS
PAGSPTSTGPS,
(SEQ ID NO: 47)
GAPGGSPAGSPTSTEEGTSESATPESGPGTSTEPSEGSA
PGSPAGSPTSTGPSGAP,
(SEQ ID NO: 48)
GGGSPAPTPTPAPTPAPTPAGGGPS,
(SEQ ID NO: 49)
GAPGGGSPAPTPTPAPTPAPTPAGGGPSGAP,
(SEQ ID NO: 50)
GGGSPAPAPTPAPAPTPAPAGGGPS,
(SEQ ID NO: 51)
GAPGGGSPAPAPTPAPAPTPAPAGGGPSGAP,
(SEQ ID NO: 52)
GGGSAEAAAKEAAAKEAAAKAGGPS,
(SEQ ID NO: 53)
GAPGGGSAEAAAKEAAAKEAAAKAGGPSGAP,
(SEQ ID NO: 54)
GGGSPAEAAAKEAAAKEAAAKEAAAKEAAAKAPSGGG,
(SEQ ID NO: 55)
GAPGGGSPAEAAAKEAAAKEAAAKEAAAKEAAAKAPSGGGGAP,
(SEQ ID NO: 56)
GGGGSGGGGSGGGGS,
(SEQ ID NO: 57)
GAPGGGGSGGGGSGGGGSGAP,
(SEQ ID NO: 58)
GGGGSGGGGSGGGGSGGGGS,
(SEQ ID NO: 59)
GAPGGGGSGGGGSGGGGSGGGGSGAP,
(SEQ ID NO: 60)
GGGGA,
(SEQ ID NO: 61)
GGGGAGGGGAGGGGAGGGGAGGGPST,
(SEQ ID NO: 62)
GGGGAGGGGAGGGGAGGGGAGGGPSH,
(SEQ ID NO: 63)
GGGGAGGGGAGGGGAGGGGAGGGPSGGGGAGGGPS,
(SEQ ID NO: 64)
GAPGGGGAGGGGAGGGGAGGGGAGGGPSGGGGAGGGPSGAP,
(SEQ ID NO: 65)
GGGGAGGGGAGGGGAGGGGAGGGPSGGGGAGGGGAGGGGAGG
GGAGGGPS,
(SEQ ID NO: 66)
GAPGGGGAGGGGAGGGGAGGGGAGGGPSGGGGAGGGGAGGGG
AGGGGAGGGPSGAP,
(SEQ ID NO: 67)
GGGGAGGGGAGGGGAGGGPSGGGGAGGGGAGGGPS,
(SEQ ID NO: 68)
GAPGGGGAGGGGAGGGGAGGGPSGGGGAGGGGAGGGPSGAP,
(SEQ ID NO: 69)
GGGGAGGGGAGGGGAGGGPSGGGGAGGGGAGGGPSGGGGAGG
GGAGGGPS,
(SEQ ID NO: 70)
GAPGGGGAGGGGAGGGGAGGGPSGGGGAGGGGAGGGPSGGGG
AGGGGAGGGPSGAP,
(SEQ ID NO: 71)
GGGGAGGGGAGGGGAGGGGAGGGPS,
(SEQ ID NO: 72)
GAPGGGGAGGGGAGGGGAGGGGAGGGPSGAP,
(SEQ ID NO: 73)
GGGGAGGGGAAAAASGGGGAGGGPS,
(SEQ ID NO: 74)
GAPGGGGAGGGGAAAAASGGGGAGGGPSGAP,
(SEQ ID NO: 75)
GGGGAGGGGAAAAASGGGGAGGGGAAAAASGGGGAGGGGAAA
AASGGGPS,
(SEQ ID NO: 76)
GAPGGGGAGGGGAAAAASGGGGAGGGGAAAAASGGGGAGGGGA
AAAASGGGPSGAP,
(SEQ ID NO: 77)
GGGGAGGGGAAAAASGGGPSGGGGAAAAASGGGPSGGGGAAAA
ASGGGPS,
(SEQ ID NO: 78)
GAPGGGGAGGGGAAAAASGGGPSGGGGAAAAASGGGPSGGGGAA
AAASGGGPSGAP,
(SEQ ID NO: 79)
GGGGAGGGGAGGGGA,
(SEQ ID NO: 80)
GAPGGGGAGGGGAGGGGAGAP,
(SEQ ID NO: 81)
GGGGAGGGGAGGGGAGGGGA,
(SEQ ID NO: 82)
GAPGGGGAGGGGAGGGGAGGGGAGAP,
GGGGAGGGGAGGGGAGGGGAGGGGAGGGGAGGGGAGGGGAGG
GPS,
[or
(SEQ ID NO: 83)
(GGGGA)₈GGGPS],
GGGGAGGGGAGGGGAGGGGAGGGGAGGGGAGGGGAGGGGAGGG
PSH
[or
(SEQ ID NO: 84)
(GGGGA)₈GGGPSH],
GGGGAGGGGAGGGGAGGGGAGGGGAGGGGAGGGGAGGGGAGGGGA
GGGPS
[or
(SEQ ID NO: 85)
(GGGGA)₉GGGPS],
GGGGAGGGGAGGGGAGGGGAGGGGAGGGGAGGGGAGGGGAGGGG
AGGGPSH
[or
(SEQ ID NO: 86)
(GGGGA)₉GGGPSH],
(SEQ ID NO: 87)
GGGGPAPGPGPAPGPAPGPAGGGPS,
(SEQ ID NO: 88)
GAPGGGGPAPGPGPAPGPAPGPAGGGPGGAP,
(SEQ ID NO: 89)
GGGGPAPAPGPAPAPGPAPAGGGPS,
and
(SEQ ID NO: 90)
GAPGGGGPAPAPGPAPAPGPAPAGGGPGGAP.

In various embodiments, the spacer peptide is selected from the group consisting of

(SEQ ID NO: 36)
GGGGSGGGGSGGGGSGGGGSGGGPS,
(SEQ ID NO: 44)
GGSPAGSPTSTEEGTSESATPESGPGTSTEPSEGSAPGSPAGS
PGPS,
(SEQ ID NO: 45)
GAPGGSPAGSPTSTEEGTSESATPESGPGTSTEPSEGSAPGSP
AGSPGPSGAP,
(SEQ ID NO: 46)
GGSPAGSPTSTEEGTSESATPESGPGTSTEPSEGSAPGSPAGSP
TSTGPS,
(SEQ ID NO: 47)
GAPGGSPAGSPTSTEEGTSESATPESGPGTSTEPSEGSAPGSPA
GSPTSTGPSGAP,
(SEQ ID NO: 48)
GGGSPAPTPTPAPTPAPTPAGGGPS,
(SEQ ID NO: 49)
GAPGGGSPAPTPTPAPTPAPTPAGGGPSGAP,
(SEQ ID NO: 50)
GGGSPAPAPTPAPAPTPAPAGGGPS,
(SEQ ID NO: 51)
GAPGGGSPAPAPTPAPAPTPAPAGGGPSGAP,
(SEQ ID NO: 52)
GGGSAEAAAKEAAAKEAAAKAGGPS,
(SEQ ID NO: 53)
GAPGGGSAEAAAKEAAAKEAAAKAGGPSGAP,
(SEQ ID NO: 54)
GGGSPAEAAAKEAAAKEAAAKEAAAKEAAAKAPSGGG,
(SEQ ID NO: 55)
GAPGGGSPAEAAAKEAAAKEAAAKEAAAKEAAAKAPSGGGGAP,
and
(SEQ ID NO: 71)
GGGGAGGGGAGGGGAGGGGAGGGPS.

In various embodiments, the spacer peptide is selected from the group consisting of

(SEQ ID NO: 36)
GGGGSGGGGSGGGGSGGGGSGGGPS,
(SEQ ID NO: 47)
GAPGGSPAGSPTSTEEGTSESATPESGPGTSTEPSEGSAPGSP
AGSPTSTGPSGAP,
(SEQ ID NO: 51)
GAPGGGSPAPAPTPAPAPTPAPAGGGPSGAP,
(SEQ ID NO: 55)
GAPGGGSPAEAAAKEAAAKEAAAKEAAAKEAAAKAPSGGGGAP,
and
(SEQ ID NO: 71)
GGGGAGGGGAGGGGAGGGGAGGGPS.

In various embodiments, the lysosomal targeting domain or IGF-II peptide tag comprises amino acids 8-67 of mature human IGF-II (SEQ ID NO: 2, 4). In various embodiments, the IGF-II peptide tag comprises a mutation at residue Arg37. In various embodiments, the mutation is a substitution of alanine for arginine. In various embodiments, the lysosomal targeting domain or IGF-II peptide tag comprises IGF2 Δ8-67 R37A.

In various embodiments, the fusion protein comprises amino acids 1-743 of human Naglu (SEQ ID NO: 1, 3). In various embodiments, the fusion protein comprises amino acids 24-743 of human Naglu.

In various embodiments, the effective amount of fusion protein is in the range of about 0.1-1 mg/kg, about 1-5 mg/kg, about 2.5-20 mg/kg, about 5-20 mg/kg, about 10-50 mg/kg, or 20-100 mg/kg of body weight of the subject. In various embodiments, the effective amount of fusion protein is about 2.5-20 mg per kilogram of body weight of the subject.

In various embodiments, the fusion protein is administered intrathecally, intravenously, intramuscularly, parenterally, transdermally, or transmucosally. In various embodiments, the fusion protein is administered intrathecally. In various embodiments, the intrathecal administration optionally further comprises administering the fusion protein intravenous

In various embodiments, intrathecal administration comprises introducing the fusion protein into a cerebral ventricle, lumbar area, or cisterna magna.

In various embodiments, the fusion protein is administered bimonthly, monthly, triweekly, biweekly, weekly, daily, or at variable intervals.

In various embodiments, the treatment results in reducing glycosaminoglycan (GAG) levels in a brain tissue. It is further contemplated that the treatment results in reducing lysosomal storage granules in a brain tissue.

Also contemplated are compositions comprising the targeted therapeutic fusion proteins as described herein for use in treating lysosomal storage diseases. Exemplary lysosomal storage diseases include those set out in Table 1.

Other features, objects, and advantages of the present invention are apparent in the detailed description that follows. It should be understood, however, that the detailed description, while indicating embodiments of the present invention, is given by way of illustration only, not limitation. Various changes and modifications within the scope of the invention will become apparent to those skilled in the art from the detailed description.

### BRIEF DESCRIPTION OF THE DRAWINGS

The drawings are for illustration purposes only, not for limitation.

FIGS. 1A-1B depict the amino acid sequences of a portion of an exemplary therapeutic fusion protein comprising (FIG. 1A) Naglu and (FIG. 1B) an IGF-II peptide comprising residues 8-67 of IGF-II and having an amino acid substitution at residue 37, R37A (Arg37Ala).

FIGS. 2A-2B depict the nucleotide sequences of a portion of an exemplary therapeutic fusion protein comprising (FIG. 2A) Naglu and (FIG. 2B) an IGF-II peptide comprising residues 8-67 of IGF-II and having an amino acid substitution at residue 37, R37A (Arg37Ala).

FIG. 3 discloses exemplary spacer sequences contemplated for use in the therapeutic fusion protein.

### DEFINITIONS

Amelioration: As used herein, the term “amelioration” is meant the prevention, reduction or palliation of a state, or improvement of the state of a subject. Amelioration includes, but does not require complete recovery or complete prevention of a disease condition. In some embodiments, amelioration includes reduction of accumulated materials inside lysosomes of relevant diseases tissues.

Furin-resistant IGF-II mutein: As used herein, the term “furin-resistant IGF-II mutein” refers to an IGF-II-based peptide containing an altered amino acid sequence that abolishes at least one native fulin protease cleavage site or changes a sequence close or adjacent to a native furin protease cleavage site such that the furin cleavage is prevented, inhibited, reduced, or slowed down as compared to a wild-type human IGF-II peptide. As used herein, a furin-resistant IGF-II mutein is also referred to as an IGF-II mutein that is resistant to furin.

Furin protease cleavage site: As used herein, the term “furin protease cleavage site” (also referred to as “furin cleavage site” or “furin cleavage sequence”) refers to the amino acid sequence of a peptide or protein that serves as a recognition sequence for enzymatic protease cleavage by furin or furin-like proteases. Typically, a furin protease cleavage site has a consensus sequence Arg-X-X-Arg (SEQ ID NO: 6), X is any amino acid. The cleavage site is positioned after the carboxy-terminal arginine (Arg) residue in the sequence. In some embodiments, a furin cleavage site may have a consensus sequence Lys/Arg-X-X-X-Lys/Arg-Arg (SEQ ID NO: 7), X is any amino acid. The cleavage site is positioned after the carboxy-terminal arginine (Arg) residue in the sequence.

Furin: As used herein, the term “furin” refers to any protease that can recognize and cleave the furin protease cleavage site as defined herein, including furin or furin-like protease. Furin is also known as paired basic amino acid cleaving enzyme (PACE). Furin belongs to the subtilisin-like proprotein convertase family. The gene encoding furin was known as FUR (FES Upstream Region).

Furin-deficient cells: As used herein, the term “furin-deficient cells” refers to any cells whose fulin protease activity is inhibited, reduced or eliminated. Furin-deficient cells include both mammalian and non-mammalian cells that do not produce furin or produce reduced amount of furin or defective furin protease.

Glycosylation Independent Lysosomal Targeting: As used herein, the term “glycosylation independent lysosomal targeting” (also referred to as “GILT”) refer to lysosomal targeting that is mannose-6-phosphate-independent.

Human Alpha-N-acetylglucosaminidase: As used herein, the term “human alpha-N-acetylglucosaminidase” (also referred to as “Naglu”) refers to precursor (i.e., containing the native Naglu signal peptide sequence) or processed (i.e., lacking the native Naglu signal peptide sequence) wild-type form of human alpha-N-acetylglucosaminidase, or a functional fragment or variant thereof, that is capable of reducing glycosaminoglycan (GAG) levels in mammalian lysosomes or that can rescue or ameliorate one or more MPS IIIB (Sanfilippo B Syndrome) symptoms. As used herein, the term “functional” as it relates to Naglu refers to a Naglu enzyme that is capable of being taken up by mammalian lysosomes and having sufficient enzymatic activity to reduce storage material, i.e., glycosaminoglycan (GAG), in the mammalian lysosome.

IGF-II mutein: As used herein, the term “IGF-II mutein” refers to an IGF-II-based peptide containing an altered amino acid sequence. As used herein, the term, “furin-resistant IGF-II mutein” refers to an IGF-II-based peptide containing an altered amino acid sequence that abolishes at least one native furin protease cleavage site or changes a sequence close or adjacent to a native furin protease cleavage site such that the furin cleavage is prevented, inhibited, reduced, or slowed down as compared to a wild-type human IGF-II peptide. As used herein, a furin-resistant IGF-II mutein is also referred to as an IGF-II mutein that is resistant to furin.

Improve, increase, or reduce: As used herein, the terms “improve,” “increase” or “reduce,” or grammatical equivalents, indicate values that are relative to a baseline measurement, such as a measurement in the same individual prior to initiation of the treatment described herein, or a measurement in a control individual (or multiple control individuals) in the absence of the treatment described herein. A “control individual” is an individual afflicted with the same form of lysosomal storage disease (e.g., MPS IIIB (Sanfilippo B Syndrome)) as the individual being treated, who is about the same age as the individual being treated (to ensure that the stages of the disease in the treated individual and the control individual(s) are comparable).

Individual, subject, patient: As used herein, the terms “subject,” “individual” or “patient” refer to a human or a non-human mammalian subject. The individual (also referred to as “patient” or “subject”) being treated is an individual (fetus, infant, child, adolescent, or adult human) suffering from a lysosomal storage disease, for example, MPS IIIB (Sanfilippo B Syndrome) (i.e., either infantile-, juvenile-, or adult-onset or severe/classical type or attenuated type MPS IIIB (Sanfilippo B Syndrome)) or having the potential to develop a lysosomal storage disease (e.g., MPS IIIB (Sanfilippo B Syndrome)).

Lysosomal storage diseases: As used herein, “lysosomal storage diseases” refer to a group of genetic disorders that result from deficiency in at least one of the enzymes (e.g., acid hydrolases) that are required to break macromolecules down to peptides, amino acids, monosaccharides, nucleic acids and fatty acids in lysosomes. As a result, individuals suffering from lysosomal storage diseases have accumulated materials in lysosomes. Exemplary lysosomal storage diseases are listed in Table 1.

Lysosomal enzyme: As used herein, the term “lysosomal enzyme” refers to any enzyme that is capable of reducing accumulated materials in mammalian lysosomes or that can rescue or ameliorate one or more lysosomal storage disease symptoms. Lysosomal enzymes suitable for the invention include both wild-type or modified lysosomal enzymes and can be produced using recombinant and synthetic methods or purified from nature sources. Exemplary lysosomal enzymes are listed in Table 1.

Spacer: As used herein, the term “spacer” (also referred to as “linker”) refers to a peptide sequence between two protein moieties in a fusion protein. A spacer is generally designed to be flexible or to interpose a structure, such as an alpha-helix, between the two protein moieties. A spacer can be relatively short, such for example, the sequence Gly-Ala-Pro (GAP) (SEQ ID NO: 9), Gly-Gly-Gly-Gly-Ser (GGGGS) (SEQ ID NO: 12), Gly-Gly-Gly-Gly-Ala (GGGGA) (SEQ ID NO: 60) or Gly-Gly-Gly-Gly-Gly-Pro (GGGGGP) (SEQ ID NO: 13), or can be longer, such as, for example, 10-25 amino acids in length, 25-50 amino acids in length or 35-55 amino acids in length. Exemplary spacer sequences are disclosed in greater detail in the Detailed Description.

Therapeutically effective amount: As used herein, the term “therapeutically effective amount” or “effective amount” refers to an amount of a targeted therapeutic fusion protein which confers a therapeutic effect on the treated subject, at a reasonable benefit/risk ratio applicable to any medical treatment. The therapeutic effect may be objective (i.e., measurable by some test or marker) or subjective (i.e., subject gives an indication of or feels an effect). In particular, the “therapeutically effective amount” refers to an amount of a therapeutic fusion protein or composition effective to treat, ameliorate, or prevent a desired disease or condition, or to exhibit a detectable therapeutic or preventative effect, such as by ameliorating symptoms associated with the disease, preventing or delaying the onset of the disease, and/or also lessening the severity or frequency of symptoms of the disease. A therapeutically effective amount is commonly administered in a dosing regimen that may comprise multiple unit doses. For any particular therapeutic fusion protein, a therapeutically effective amount (and/or an appropriate unit dose within an effective dosing regimen) may vary, for example, depending on route of administration, on combination with other pharmaceutical agents. Also, the specific therapeutically effective amount (and/or unit dose) for any particular patient may depend upon a variety of factors including the disorder being treated and the severity of the disorder; the activity of the specific pharmaceutical agent employed; the specific composition employed; the age, body weight, general health, sex and diet of the patient; the time of administration, route of administration, and/or rate of excretion or metabolism of the specific fusion protein employed; the duration of the treatment; and like factors as is well known in the medical arts.

Treatment: As used herein, the term “treatment” (also “treat” or “treating”) refers to any administration of a therapeutic fusion protein or pharmaceutical composition comprising said therapeutic fusion protein that partially or completely alleviates, ameliorates, relieves, inhibits, delays onset of, reduces severity of and/or reduces incidence of one or more symptoms or features of a particular disease, disorder, and/or condition. Such treatment may be of a subject who does not exhibit signs of the relevant disease, disorder and/or condition and/or of a subject who exhibits only early signs of the disease, disorder, and/or condition. Alternatively or additionally, such treatment may be of a subject who exhibits one or more established signs of the relevant disease, disorder and/or condition. For example, treatment can refer to improvement of cardiac status (e.g., increase of end-diastolic and/or end-systolic volumes, or reduction, amelioration or prevention of the progressive cardiomyopathy that is typically found in, e.g., Pompe disease) or of pulmonary function (e.g., increase in crying vital capacity over baseline capacity, and/or normalization of oxygen desaturation during crying); improvement in neurodevelopment and/or motor skills (e.g., increase in AIMS score); reduction of storage (e.g., glycosaminoglycan (GAG), levels in tissue of the individual affected by the disease; or any combination of these effects. In some embodiments, treatment includes improvement of glycosaminoglycan (GAG) clearance, particularly in reduction or prevention of MPS IIIB (Sanfilippo B Syndrome)-associated neuronal symptoms.

As used in this application, the terms “about” and “approximately” are used as equivalents. Any numerals used in this application with or without about/approximately are meant to cover any normal fluctuations appreciated by one of ordinary skill in the relevant art.

### DETAILED DESCRIPTION OF THE INVENTION

The present invention provides improved methods and compositions for targeting lysosomal enzymes based on the glycosylation-independent lysosomal targeting (GILT) technology. Among other things, the present invention provides IGF-II muteins that are resistant to furin and/or has reduced or diminished binding affinity for the insulin receptor, and/or has reduced or diminished binding affinity for the IGF-I receptor and targeted therapeutic fusion proteins containing an IGF-II mutein of the invention. The present invention also provides methods of making and using the same.

Various aspects of the invention are described in detail in the following sections. The use of sections is not meant to limit the invention. Each section can apply to any aspect of the invention. In this application, the use of “or” means “and/or” unless stated otherwise.

### Lysosomal Enzymes

A lysosomal enzyme suitable for the invention includes any enzyme that is capable of reducing accumulated materials in mammalian lysosomes or that can rescue or ameliorate one or more lysosomal storage disease symptoms. Suitable lysosomal enzymes include both wild-type or modified lysosomal enzymes and can be produced using recombinant or synthetic methods or purified from natural sources. Exemplary lysosomal enzymes are listed in Table 1.

TABLE 1
Lysosomal Storage Diseases and associated enzyme defects

| | | |
|---|---|---|
| Disease Name | Enzyme Defect | Substance Stored |

A. Glycogenosis Disorders

| | | |
|---|---|---|
| Pompe Disease | Acid-al, 4-Glucosidase | Glycogen al-4 linked |
| | | Oligosaccharides |

B. Glycolipidosis Disorders

| | | |
|---|---|---|
| GM 1 Gangliodsidosis | 13-Galactosidase | GM₁ Gangliosides |
| Tay-Sachs Disease | 13-Hexosaminidase A | GM₂ Ganglioside |
| GM2 Gangliosidosis: AB | GM₂ Activator Protein | GM₂ Ganglioside |
| Variant | | |
| Sandhoff Disease | 13-Hexosaminidase A&B | GM₂ Ganglioside |
| Fabry Disease | a-Galactosidase A | Globosides |
| Gaucher Disease | Glucocerebrosidase | Glucosylceramide |
| Metachromatic | Arylsulfatase A | Sulphatides |
| Leukodystrophy | | |
| Krabbe Disease | Galactosylceramidase | Galactocerebroside |
| Niemann-Pick, Types A & B | Acid Sphingomyelinase | Sphingomyelin |
| Niemann-Pick, Type C | Cholesterol Esterification | Sphingomyelin |
| | Defect | |
| Niemann-Pick, Type D | Unknown | Sphingomyelin |
| Farber Disease | Acid Ceramidase | Ceramide |
| Wolman Disease | Acid Lipase | Cholesteryl Esters |

C. Mucopolysaccharide Disorders

| | | |
|---|---|---|
| Hurler Syndrome (MPS IH) | α-L-Iduronidase | Heparan & Dermatan |
| | | Sulfates |
| Scheie Syndrome (MPS IS) | α-L-Iduronidase | Heparan & Dermatan |
| | | Sulfates |
| Hurler-Scheie (MPS IH/S) | α-L-Iduronidase | Heparan & Dennatan |
| | | Sulfates |
| Hunter Syndrome (MPS II) | Iduronate Sulfatase | Heparan & Dermatan |
| | | Sulfates |
| Sanfilippo A (MPS IliA) | Heparan N-Sulfatase | Heparan Sulfate |
| Sanfilippo B (MPS IIIB) | α-N-Acetylglucosaminidase | Heparan Sulfate |
| Sanfilippo C (MPS IIIC) | Acetyl-CoA-Glucosaminide | Heparan Sulfate |
| | Acetyltransferase | |
| Sanfilippo D (MPS IIID) | N-Acetylglucosamine-6- | Heparan Sulfate |
| | Sulfatase | |
| Morquio A (MPS NA) | Galactosamine-6-Sulfatase | Keratan Sulfate |
| Morquio B (MPS IVB) | 13-Galactosidase | Keratan Sulfate |
| Maroteaux-Lamy (MPS VI) | Arylsulfatase B | Dermatan Sulfate |
| Sly Syndrome (MPS VII) | 13-Glucuronidase | |

D. Oligosaccharide/Glycoprotein Disorders

| | | |
|---|---|---|
| α-Mannosidosis | α-Mannosidase | Mannose/Oligosaccharides |
| β-Mannosidosis | β-Mannosidase | Mannose/Oligosaccharides |
| Fucosidosis | α-L-Fucosidase | Fucosyl Oligosaccharides |
| Aspartylglucosaminuria | N-Asparty1-J3- | Aspartylglucosamine |
| | Glucosaminidase | Asparagines |
| Sialidosis (Mucolipidosis I) | α-Neuraminidase | SialyIoligosaccharides |
| Galactosialidosis | Lysosomal Protective | Sialyloligosaccharides |
| (Goldberg Syndrome) | Protein Deficiency | |
| Schindler Disease | α-N-Acetyl- | |
| | Galactosaminidase | |

E. Lysosomal Enzyme Transport Disorders

| | | |
|---|---|---|
| Mucolipidosis II (I-Cell | N-Acetylglucosamine-1- | Heparan Sulfate |
| Disease) | Phosphotransferase | |
| Mucolipidosis III (Pseudo- | Same as ML II | |
| Hurler Polydystrophy) | | |

F. Lysosomal Membrane Transport Disorders

| | | |
|---|---|---|
| Cystinosis | Cystine Transport Protein | Free Cystine |
| Salla Disease | Sialic Acid Transport Protein | Free Sialic Acid and |
| | | Glucuronic Acid |
| Infantile Sialic Acid Storage | Sialic Acid Transport Protein | Free Sialic Acid and |
| Disease | | Glucuronic Acid |

G. Other

| | | |
|---|---|---|
| Batten Disease | Unknown | Lipofuscins |
| (Juvenile Neuronal Ceroid | | |
| Lipofuscinosis) | | |
| Infantile Neuronal Ceroid | Palmitoyl-Protein Thioesterase | Lipofuscins |
| Lipofuscinosis | | |
| Late Infantile Neuronal | Tripeptidyl Peptidase I | Lipofuscins |
| Ceroid Lipofuscinosis | | |
| Mucolipidosis IV | Unknown | Gangliosides & Hyaluronic |
| | | Acid |
| Prosaposin | Saposins A, B, C or D | |

In some embodiments, a lysosomal enzyme contemplated herein includes a polypeptide sequence having 50-100%, including 50%, 55%, 60%, 65%, 70%, 71%, 72%, 73%, 74%, 75%, 760%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% and 100%, sequence identity to the naturally-occurring polynucleotide sequence of a human enzyme shown in Table 1, while still encoding a protein that is functional, i.e., capable of reducing accumulated materials, e.g., glycosaminoglycan (GAG), in mammalian lysosomes or that can rescue or ameliorate one or more lysosomal storage disease symptoms.

“Percent (%) amino acid sequence identity” with respect to the lysosomal enzyme sequences is defined as the percentage of amino acid residues in a candidate sequence that are identical with the amino acid residues in the naturally-occurring human enzyme sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, ALIGN or Megalign (DNASTAR) software. Those skilled in the at 1 can determine appropriate parameters for measuring alignment, including any algorithms needed to achieve maximal alignment over the full length of the sequences being compared. Preferably, the WU-BLAST-2 software is used to determine amino acid sequence identity (Altschul et al., Methods in Enzymology 266, 460-480 (1996). WU-BLAST-2 uses several search parameters, most of which are set to the default values. The adjustable parameters are set with the following values: overlap span=1, overlap fraction=0.125, world threshold (T)=11. HSP score (S) and HSP S2 parameters are dynamic values and: n e established by the program itself, depending upon the composition of the particular sequence, however, the minimum values may be adjusted and are set as indicated above.

### Alpha-N-acetylglucosaminidase

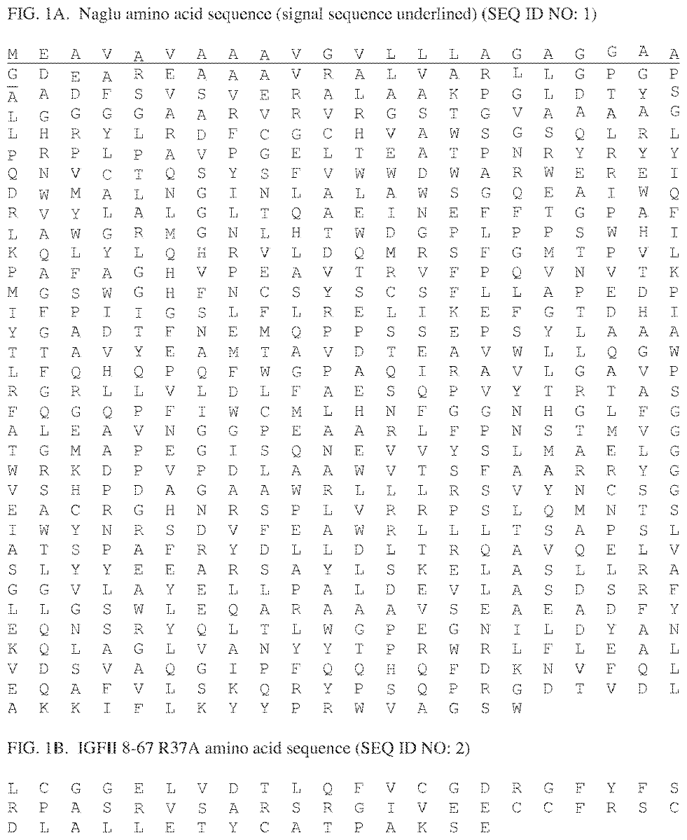

Alpha-N-acetylglucosaminidase, Naglu, is produced as a precursor molecule that is processed to a mature form. This process generally occurs by removing the 23 amino acid signal peptide as the protein enters the endoplasmic reticulum. Typically, the precursor form is also referred to as full-length precursor or full-length Naglu protein, which contains 743 amino acids (SEQ ID NO: 1). The N-terminal 23 amino acids are cleaved as the precursor protein enters the endoplasmic reticulum, resulting in a processed or mature form. Thus, it is contemplated that the N-terminal 23 amino acids are generally not required for the Naglu protein activity. The amino acid sequences of the mature form and full-length precursor form of a typical wild-type or naturally-occurring human Naglu protein are shown in FIG. 1 and set out in SEQ ID NO: 1. The nucleotide sequence of the coding region of human Naglu is set out in SEQ ID NO: 3. The mRNA sequence of human Naglu is described in Genbank Accession number NM_000263. In various embodiments, the Naglu is human Naglu, with (amino acids 1-743) or without (amino acids 24-743) signal sequence.

U.S. Pat. No. 6,255,096 describes that the molecular weight of purified human alpha-N-acetylglucosaminidase (i.e. 82 kDa and 77 kDa) and recombinant mammalian alpha-N-acetylglucosaminidase produced in CHO cells (i.e. 89 kDa and 79 kDa) are greater than the deduced molecular weight of the Naglu polypeptide (i.e. 70 kDa), suggesting that the purified and recombinant polypeptide are post-translationally modified. See also Weber et al., Hum Mol Genet 5:771-777, 1996.

### Mucopolysaccharidosis III B (Sanfilippo B Syndrome)

One exemplary lysosomal storage disease is Mucopolysaccharidosis III B (MPS IIIB) disease, also known as Sanfilippo Type B Syndrome. MPS IIIB, Sanfilippo B Syndrome, is a rare autosomal recessive genetic disorder that is characterized by a deficiency of the enzyme alpha-N-acetyl-glucosaminidase (Naglu). In the absence of this enzyme, glycosaminoglycans (GAG), for example the GAG heparan sulfate, and pmlially degraded GAG molecules cannot be cleared from the body and accumulate in lysosomes of various tissues, resulting in progressive widespread somatic dysfunction (Kakkis et al., N Engl J Med. 344(3):182-8, 2001). It has been shown that GAGs accumulate in lysosomes of neurons and glial cells, with lesser accumulation outside the brain.

Four distinct forms of MPS III, designated MPS IliA, B, C, and D, have been identified. Each represents a deficiency in one of four enzymes involved in the degradation of the GAG heparan sulfate (Table 1). All forms include varying degrees of the same clinical symptoms, including com se facial features, hepatosplenomegaly, corneal clouding and skeletal defonnities. Most notably, however, is the severe and progressive loss of cognitive ability, which is tied not only to the accumulation of heparan sulfate in neurons, but also the subsequent elevation of the gangliosides GM2, GM3 and GD2 caused by primary GAG accumulation (Walkley et al., Ann NY Acad Sci. 845:188-99, 1998).

A characteristic clinical feature of Sanfilippo B Syndrome is central nervous system (CNS) degeneration, which results in loss of, or failure to attain, major developmental milestones. The progressive cognitive decline culminates in dementia and premature mortality. The disease typically manifests itself in young children, and the lifespan of an affected individual generally does not extend beyond late teens to early twenties.

MPS III diseases all have similar symptoms that typically manifest in young children. Affected infants are apparently normal, although some mild facial dysmorphism may be noticeable. The stiff joints, hirsuteness and coarse hair typical of other mucopolysaccharidoses are usually not present until late in the disease. After an initial symptom-free interval, patients usually present with a slowing of development and/or behavioral problems, followed by progressive intellectual decline resulting in severe dementia and progressive motor disease. Acquisition of speech is often slow and incomplete. The disease progresses to increasing behavioral disturbance including temper tantrums, hyperactivity, destructiveness, aggressive behavior, pica and sleep disturbance. As affected children have normal muscle strength and mobility, the behavioral disturbances are very difficult to manage. In the final phase of the illness, children become increasingly immobile and unresponsive, often require wheelchairs, and develop swallowing difficulties and seizures. The life-span of an affected child does not usually extend beyond late teens to early twenties.

An alpha-N-acetylglucosaminidase enzyme suitable for treating MPS IIIB (Sanfilippo B Syndrome) includes a wild-type human alpha-N-acetylglucosaminidase (SEQ ID NO: 1 or 3), or a functional fragment or sequence variant thereof which retains the ability to be taken up into mammalian lysosomes and to hydrolyze alpha, 1,4 linkages at the terminal N-acetyl-D-glucosamine residue in linear oligosaccharides.

Efficacy of treatment of MPS IIIB (Sanfilippo B Syndrome) using recombinant targeted therapeutic fusion proteins as described herein can be measured using techniques known in the art, as well as by analysis of lysosomal and neuronal biomarkers. Initial experiments are conducted on Naglu knock-out animals (see Li et al., Proc Natl Acad Sci USA 96:14505-510, 1999). Naglu knockouts present with large amounts of heparan sulfate in the liver and kidney and elevation of gangliosides in brain.

Assays include analysis of the activity of and biodistribution of the exogenous enzyme, reduction of GAG storage in the lysosomes, particularly in brain cells, and activation of astrocytes and microglia. Levels of various lysosomal or neuronal biomarkers include, but are not limited to, Lysosomal-associated membrane protein 1 (LAMP1), glypican, gangliosides, cholesterol, Subunit c of Mitochondrial ATP Synthase (SCMAS), ubiquitin, P-GSK3b, beta amyloid and P-tau. Survival and behavioral analysis is also performed using techniques known in the field.

Experiments have shown that Subunit c of Mitochondrial ATP Synthase (SCMAS) protein accumulates in the lysosomes of MPS IIIB animals (Ryazantsev et al., Mol Genet Metab. 90(4): 393-401, 2007). LAMP-1 and GM130 have also been shown to be elevated in MPS IIIB animals (Vitry et al., Am J Pathol. 177(6):2984-99, 2010).

In various embodiments, treatment of a lysosomal storage disease refers to decreased lysosomal storage (e.g., of GAG) in various tissues. In various embodiments, treatment refers to decreased lysosomal storage in brain target tissues, spinal cord neurons, and/or peripheral target tissues. In certain embodiments, lysosomal storage is decreased by about 5%, 10%, 15%, 20%, 25%, 30%, 35%, 40%, 45%, 50%, 55%, 60%, 65%, 70%, 75%, 80%, 85%, 90%, 95%, 100% or more as compared to a control. In various embodiments, lysosomal storage is decreased by at least 1-fold, 2-fold, 3-fold, 4-fold, 5-fold, 6-fold, 7-fold, 8-fold, 9-fold, 10-fold or more as compared to a control.

In various embodiments, treatment refers to increased enzyme activity in various tissues. In various embodiments, treatment refers to increased enzyme activity in brain target tissues, spinal cord neurons and/or peripheral target tissues. In various embodiments, enzyme activity is increased by about 5%0, 1%, 15%, 20%, 25%, 30%, 35%, 40%, 45%, 50%, 55%, 60%, 65%, 70%, 75%, 80%, 85%, 90%, 95%, 100%, 200%, 300%, 400%, 500%, 600%, 700%, 800%, 900% 1000% or more as compared to a control. In various embodiments, enzyme activity is increased by at least 1-fold, 2-fold, 3-fold, 4-fold, 5-fold, 6-fold, 7-fold, 8-fold, 9-fold, 10-fold or more as compared to a control. In various embodiments, increased enzymatic activity is at least approximately 10 nmol/hr/mg, 20 nmol/hr/mg, 40 nmol/hr/mg, 50 nmol/hr/mg, 60 nmol/hr/mg, 70 nmol/hr/mg, 80 nmol/hr/mg, 90 nmol/hr/mg, 100 nmol/hr/mg, 150 nmol/hr/mg, 200 nmol/hr/mg, 250 nmol/hr/mg, 300 nmol/hr/mg, 350 nmol/hr/mg, 400 nmol/hr/mg, 450 nmol/hr/mg, 500 nmol/hr/mg, 550 nmol/hr/mg, 600 nmol/hr/mg or more. In various embodiments, the lysosomal enzyme is Naglu.

### Enzyme Replacement Therapy

Enzyme replacement therapy (ERT) is a therapeutic strategy to correct an enzyme deficiency by infusing the missing enzyme into the bloodstream. As the blood perfuses patient tissues, enzyme is taken up by cells and transported to the lysosome, where the enzyme acts to eliminate material that has accumulated in the lysosomes due to the enzyme deficiency. For lysosomal enzyme replacement therapy to be effective, the therapeutic enzyme must be delivered to lysosomes in the appropriate cells in tissues where the storage defect is manifest. Conventional lysosomal enzyme replacement therapeutics are delivered using carbohydrates naturally attached to the protein to engage specific receptors on the surface of the target cells. One receptor, the cation-independent M6P receptor (CI-MPR), is particularly useful for targeting replacement lysosomal enzymes because the CI-MPR is present on the surface of most cell types.

The terms “cation-independent mannose-6-phosphate receptor (CI-MPR),” “M6P/IGF-II receptor,” “CI-MPR/IGF-11 receptor,” “IGF-II receptor” or “IGF2 Receptor,” or abbreviations thereof, are used interchangeably herein, referring to the cellular receptor which binds both M6P and IGF-II.

### Combination Therapy to Tolerize Subject to Enzyme Replacement Therapy

It has been found that during administration of agents such as recombinant proteins and other therapeutic agents, a subject can mount an immune response against these agents, leading to the production of antibodies that bind and interfere with the therapeutic activity as well as cause acute or chronic immunologic reactions. This problem is most significant for protein therapeutics because proteins are complex antigens and in many cases, the subject is immunologically naive to the antigens. Thus, in certain aspects of the present invention, it may be useful to render the subject receiving the therapeutic enzyme tolerant to the enzyme replacement therapy. In this context, the enzyme replacement therapy may be given to the subject as a combination therapy with a tolerizing regimen.

U.S. Pat. No. 7,485,314 (incorporated herein by reference) discloses treatment of lysosomal storage disorders using immune tolerance induction. Briefly, use of such a tolerization regimen may be useful to prevent the subject mounting an immune response to the enzyme replacement therapy and thereby decreasing or otherwise rendering ineffective the potential beneficial effects of the enzyme replacement therapy.

In one method, the invention contemplates reducing or preventing a clinically significant antigen-specific immune response to recombinant therapeutic fusion protein, for example, comprising Naglu, used to treat a lysosomal storage disorder, for example mucopolysaccharidosis IIIB (MPS IIIB or Sanfilippo B Syndrome), where the fusion protein is administered intrathecally. The method employs an initial 30-60 day regimen of aT-cell immunosuppressive agent such as cyclosporin A (CsA) and an antiproliferative agent, such as, azathioprine (Aza), combined with weekly intrathecal infusions of low doses of the enzyme, e.g., Naglu. The typical strong IgG response to weekly infusions of enzyme becomes greatly reduced or prevented using a 60 day regimen of immunosuppressive drugs, cyclosporin A (CsA) and azathioprine (Aza), combined with weekly intrathecal or intravenous infusions of low doses of fusion protein comprising enzyme. Using such tolerization regimens, it will be possible to render the subject tolerant to higher therapeutic doses of therapeutic fusion protein for up to 6 months without an increase in antibody titer against Naglu, or indeed any other enzyme that could be used for enzyme replacement of a lysosomal storage disease. Such tolerization regimens have been described in U.S. Pat. No. 7,485,314.

### Glycasylation Independent Lysosomal Targeting

A Glycosylation Independent Lysosomal I.argeting (GILT) technology was developed to target therapeutic enzymes to lysosomes. Specifically, the GILT technology uses a peptide tag instead of M6P to engage the CI-MPR for lysosomal targeting. Typically, a GILT tag is a protein, peptide, or other moiety that binds the CI-MPR in a mannose-6-phosphate-independent manner. Advantageously, this technology mimics the normal biological mechanism for uptake of lysosomal enzymes, yet does so in a manner independent of mannose-6-phosphate.

A preferred GILT tag is derived from human insulin-like growth factor II (IGF-11). Human IGF-11 is a high affinity ligand for the CI-MPR, which is also referred to as IGF-11 receptor. Binding of GILT-tagged therapeutic enzymes to the M6P/IGF-II receptor targets the protein to the lysosome via the endocytic pathway. This method has numerous advantages over methods involving glycosylation including simplicity and cost effectiveness, because once the protein is isolated, no further modifications need be made.

Detailed description of the GILT technology and GILT tag can be found in U.S. Publication Nos. 20030082176, 20040006008, 20040005309, and 20050281805, the teachings of all of which are hereby incorporated by references in their entireties.

### Furin-Resistant GILT Tag

During the course of development of GILT-tagged lysosomal enzymes for treating lysosomal storage disease, it has become apparent that the IGF-11 derived GILT tag may be subjected to proteolytic cleavage by furin during production in mammalian cells (see the examples section). Furin protease typically recognizes and cleaves a cleavage site having a consensus sequence Arg-X-X-Arg (SEQ ID NO: 6), X is any amino acid. The cleavage site is positioned after the carboxy-terminal arginine (Arg) residue in the sequence. In some embodiments, a furin cleavage site has a consensus sequence Lys/Arg-X-X-X-Lys/Arg-Arg (SEQ ID NO: 7), X is any amino acid. The cleavage site is positioned after the carboxy-terminal arginine (Arg) residue in the sequence. As used herein, the term “furin” refers to any protease that can recognize and cleave the furin protease cleavage site as defined herein, including furin or furin-like protease. Furin is also known as paired basic amino acid cleaving enzyme (PACE). Furin belongs to the subtilisin-like proprotein convertase family that includes PC3, a protease responsible for maturation of proinsulin in pancreatic islet cells. The gene encoding furin was known as FUR (FES Upstream Region).

The mature human IGF-II peptide sequence is shown below.

(SEQ ID NO: 5)
AYRPSETLCGGELVDTLQFVCGDRGFYFSRPASRVS
RtRSRtGIVEECCFRSCDLALLETYC ATPAKSE

As can be seen, the mature human IGF-II contains two potential overlapping furin cleavage sites between residues 34-40 (bolded and underlined). Arrows are inserted at two potential furin cleavage positions.

Modified GILT tags that are resistant to cleavage by furin and still retain ability to bind to the CI-MPR in a mannose-6-phosphate-independent manner are disclosed in US 20110223147. Specifically, furin-resistant GILT tags can be designed by mutating the amino acid sequence at one or more furin cleavage sites such that the mutation abolishes at least one fmin cleavage site. Thus, in some embodiments, a furin-resistant GILT tag is a furin-resistant IGF-II mutein containing a mutation that abolishes at least one furin protease cleavage site or changes a sequence adjacent to the furin protease cleavage site such that the furin cleavage is prevented, inhibited, reduced or slowed down as compared to a wild-type IGF-II peptide (e.g., wild-type human mature IGF-II). Typically, a suitable mutation does not impact the ability of the furin-resistant GILT tag to bind to the human cation-independent mannose-6-phosphate receptor. In particular, a furin-resistant IGF-II mutein suitable for the invention binds to the human cation-independent mannose-6-phosphate receptor in a mannose-6-phosphate-independent manner with a dissociation constant of 10⁻⁷ M or less (e.g., 10⁻⁸, 10⁻², 10⁻¹⁰, 10⁻¹¹, or less) at pH 7.4. In some embodiments, a furin-resistant IGF-II mutein contains a mutation within a region corresponding to amino acids 30-40 (e.g., 30-40, 31-40, 32-40, 33-40, 34-40, 30-39, 31-39, 32-39, 34-37, 33-39, 34-39, 35-39, 36-39, 37-40) of mature human IGF-II. In some embodiments, a suitable mutation abolishes at least one furin protease cleavage site. A mutation can be amino acid substitutions, deletions, insertions. For example, any one amino acid within the region corresponding to residues 30-40 (e.g., 30-40, 31-40, 32-40, 33-40, 34-40, 30-39, 31-39, 32-39, 34-37, 33-39, 34-39, 35-39, 36-39, 37-40) of SEQ ID NO:5 can be substituted with any other amino acid or deleted. For example, substitutions at position 34 may affect furin recognition of the first cleavage site. Insertion of one or more additional amino acids within each recognition site may abolish one or both furin cleavage sites. Deletion of one or more of the residues in the degenerate positions may also abolish both furin cleavage sites.

In various embodiments, a furin-resistant IGF-II mutein contains amino acid substitutions at positions corresponding to Arg37 or Arg40 of mature human IGF-II. In some embodiments, a furin-resistant IGF-II mutein contains a Lys or Ala substitution at positions Arg37 or Arg40. Other substitutions are possible, including combinations of Lys and/or Ala mutations at both positions 37 and 40, or substitutions of amino acids other than Lys or Ala.

In various embodiments, an IGF-II mutein suitable for use herein may contain additional mutations. For example, up to 30% or more of the residues of SEQ ID NO:1 may be changed (e.g., up to 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30% or more residues may be changed). Thus, an IGF-II mutein suitable for use herein may have an amino acid sequence at least 70%, including at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more identical to mature human IGF-II.

In various embodiments, an IGF-II mutein suitable for use herein is targeted specifically to the CI-MPR. Particularly useful are mutations in the IGF-II polypeptide that result in a protein that binds the CI-MPR with high affinity (e.g., with a dissociation constant of 10⁻⁷M or less at pH 7.4) while binding other receptors known to be bound by IGF-II with reduced affinity relative to native IGF-II. For example, a furin-resistant IGF-II mutein suitable for the invention can be modified to have diminished binding affinity for the IGF-I receptor relative to the affinity of naturally-occurring human IGF-II for the IGF-I receptor. For example, substitution of IGF-II residues Tyr 27 with Leu, Leu 43 with Val or Ser 26 with Phe diminishes the affinity of IGF-II for the IGF-I receptor by 94-, 56-, and 4-fold respectively (Torres et al. (1995) J. Mol. Biol.: 248(2):385-401). Deletion of residues 1-7 of human IGF-II resulted in a 30-fold decrease in affinity for the human IGF-I receptor and a concomitant 12 fold increase in affinity for the rat IGF-II receptor (Hashimoto et al. (1995) J. Biol. Chem. 270(30):18013-8). The NMR structure of iGF-II shows that Thr-7 is located near residues Phe-48 Phe and Ser-50 as well as near the Cys-9-Cys-47 disulfide bridge. It is thought that interaction of Thr-7 with these residues can stabilize the flexible N-terminal hexapeptide required for IGF-I receptor binding (Terasawa et al. (1994) EMBO J. 13(23)5590-7). At the same time this interaction can modulate binding to the IGF-II receptor. Truncation of the C-terminus of IGF-II (residues 62-67) also appear to lower the affinity of IGF-II for the IGF-I receptor by 5 fold (Roth et al. (1991) Biochem. Biophys. Res. Commun. 181(2):907-14).

The binding surfaces for the IGF-I and cation-independent M6P receptors are on separate faces of IGF-II. Based on structural and mutational data, functional cation-independent M6P binding domains can be constructed that are substantially smaller than human IGF-II. For example, the amino terminal amino acids (e.g., 1-7 or 2-7) and/or the carboxy terminal residues 62-67 can be deleted or replaced. Additionally, amino acids 29-40 can likely be eliminated or replaced without altering the folding of the remainder of the polypeptide or binding to the cation-independent M6P receptor. Thus, a targeting moiety including amino acids 8-28 and 41-61 can be constructed. These stretches of amino acids could perhaps be joined directly or separated by a linker. Alternatively, amino acids 8-28 and 41-61 can be provided on separate polypeptide chains. Comparable domains of insulin, which is homologous to IGF-II and has a tertiary structure closely related to the structure of IGF-II, have sufficient structural information to permit proper refolding into the appropriate tertiary structure, even when present in separate polypeptide chains (Wang et al. (1991) Trends Biochem. Sci. 279-281). Thus, for example, amino acids 8-28, or a conservative substitution variant thereof, could be fused to a lysosomal enzyme; the resulting fusion protein could be admixed with amino acids 41-61, or a conservative substitution variant thereof, and administered to a patient.

IGF-II can also be modified to minimize binding to serum IGF-binding proteins (Baxter (2000) Am. J. Physiol Endocrinol Metab. 278(6):967-76) to avoid sequestration of IGF-IIIGILT constructs. A number of studies have localized residues in IGF-II necessary for binding to IGF-binding proteins. Constructs with mutations at these residues can be screened for retention of high affinity binding to the M6P/IGF-II receptor and for reduced affinity for IGF-binding proteins. For example, replacing Phe-26 of IGF-II with Ser is reported to reduce affinity of IGF-II for IGFBP-1 and -6 with no effect on binding to the M6P/IGF-II receptor (Bach et al. (1993) J. Biol. Chem. 268(13):9246-54). Other substitutions, such as Lys for Glu-9, can also be advantageous. The analogous mutations, separately or in combination, in a region of IGF-I that is highly conserved with IGF-II result in large decreases in IGF-BP binding (Magee et al. (1999) Biochemistry 38(48):15863-70).

An alternate approach is to identify minimal regions of IGF-II that can bind with high affinity to the M6P/IGF-II receptor. The residues that have been implicated in IGF-II binding to the M6P/IGF-II receptor mostly cluster on one face of IGF-II (Terasawa et al. (1994) EMBO J. 13(23):5590-7). Although IGF-II tertiary structure is normally maintained by three intramolecular disulfide bonds, a peptide incorporating the amino acid sequence on the M6P/IGF-II receptor binding surface of IGF-II can be designed to fold properly and have binding activity. Such a minimal binding peptide is a highly preferred lysosomal targeting domain. For example, a preferred lysosomal targeting domain is amino acids 8-67 of human IGF-II. Designed peptides, based on the region around amino acids 48-55, which bind to the M6P/IGF-II receptor, are also desirable lysosomal targeting domains. Alternatively, a random library of peptides can be screened for the ability to bind the M6P/IGF-II receptor either via a yeast two hybrid assay, or via a phage display type assay.

### Binding Affinity for the Insulin Receptor

Many IGF-II muteins, including furin-resistant IGF-II muteins, described herein have reduced or diminished binding affinity for the insulin receptor. Thus, in some embodiments, a peptide tag suitable for the invention has reduced or diminished binding affinity for the insulin receptor relative to the affinity of naturally-occurring human IGF-II for the insulin receptor. In some embodiments, peptide tags with reduced or diminished binding affinity for the insulin receptor suitable for the invention include peptide tags having a binding affinity for the insulin receptor that is more than 1.5-fold, 2-fold, 3-fold, 4-fold, 5-fold, 6-fold, 7-fold, 8-fold, 9-fold, 10-fold, 12-fold, 14-fold, 16-fold, 18-fold, 20-fold, 30-fold, 40-fold, 50-fold, 60-fold, 70-fold, 80-fold, 90-fold, or 100-fold less than that of the wild-type mature human IGF-II. The binding affinity for the insulin receptor can be measured using various in vitro and in vivo assays known in the art. Exemplary binding assays are described in the Examples section.

### Mutagenesis

IGF-II muteins can be prepared by introducing appropriate nucleotide changes into the IGF-TT DNA, or by synthesis of the desired IGF-TT polypeptide. Variations in the IGF-TT sequence can be made, for example, using any of the techniques and guidelines for conservative and non-conservative mutations set forth, for instance, in U.S. Pat. No. 5,364,934. Variations may be a substitution, deletion or insertion of one or more codons encoding IGF-II that results in a change in the amino acid sequence of IGF-II as compared with a naturally-occurring sequence of mature human IGF-II. Amino acid substitutions can be the result of replacing one amino acid with another amino acid having similar structural and/or chemical properties, such as the replacement of a leucine with a serine, i.e., conservative amino acid replacements. Amino acid substitutions can also be the result of replacing one amino acid with another amino acid having dis-similar structural and/or chemical properties, i.e., non-conservative amino acid replacements. Insertions or deletions may optionally be in the range of 1 to 5 amino acids. The variation allowed may be determined by systematically making inse11 ions, deletions or substitutions of amino acids in the sequence and testing the resulting variants for activity in the in vivo or in vitro assays known in the art (such as binding assays to the CI-MPR or furin cleavage assays).

Scanning amino acid analysis can also be employed to identify one or more amino acids along a contiguous sequence. Among the preferred scanning amino acids aTe relatively small, neutral amino acids. Such amino acids include alanine, glycine, serine, and cysteine. Alanine is typically a preferred scanning amino acid among this group because it eliminates the side-chain beyond the beta-carbon and is less likely to alter the main-chain conformation of the variant. Alanine is also typically preferred because it is the most common amino acid. Further, it is frequently found in both buried and exposed positions [Creighton, The Proteins, (W. H. Freeman & Co., N.Y.); Chothia, J. Mol. Biol., 150:1 (1976)]. If alanine substitution does not yield adequate amounts of variant, an isoteric amino acid can be used.

The variations can be made using methods known in the art such as oligonucleotide-mediated (site-directed) mutagenesis, alanine scanning, and PCR mutagenesis. Site-directed mutagenesis [Carter et al., Nucl. Acids Res., 13:4331 (1986); Zoller et al., Nucl. Acids Res., 10:6487 (1987)], cassette mutagenesis [Wells et al., Gene, 34:315 (1985)], restriction selection mutagenesis [Wells et al., Philos. Trans. R. Soc. London SerA, 317:415 (1986)] or other known techniques can be performed on the cloned DNA to produce IGF-II muteins.

### Spacer

A GILT tag can be fused to the N-terminus or C-terminus of a lysosomal enzyme. The GILT tag can be fused directly to the lysosomal enzyme or can be separated from the lysosomal enzyme by a linker or a spacer. An amino acid linker or spacer is generally designed to be rigid, flexible or to interpose a structure, such as an alpha-helix, between the two protein moieties. A linker or spacer can be relatively short, such as, for example, the sequence Gly-Ala-Pro (GAP) (SEQ ID NO: 9), Gly-Gly-Gly-Gly-Ala (GGGGA) (SEQ ID NO: 60) or Gly-Gly-Gly-Gly-Ser (GGGGS) (SEQ ID NO: 12), or can be longer, such as, for example, 10-25 amino acids in length, 25-50 amino acids in length or 35-55 amino acids in length. The site of a fusion junction should be selected with care to promote proper folding and activity of both fusion partners and to prevent premature separation of a peptide tag from the lysosomal enzyme, e.g., alpha-N-acetylglucosaminidase.

In various embodiments, the spacer peptide comprises one or more GGGPS (SEQ ID NO: 14) or GGGSP (SEQ ID NO: 15) amino acid sequences, and optionally further comprises one or more of (i) GAP (SEQ ID NO: 9), (ii) GGGGS (SEQ ID NO: 12), (iii) GGGS (SEQ ID NO: 16), (iv) AAAAS (SEQ ID NO: 17), (v) AAAS (SEQ ID NO: 18), (vi) PAPA (SEQ ID NO: 19), (vii) TPAPA (SEQ ID NO: 20), (viii) AAAKE (SEQ ID NO: 21) or (ix) GGGGA (SEQ ID NO: 60). In various embodiments, the spacer comprises the amino acid sequence GAP (SEQ ID NO: 9), GPS (SEQ ID NO: 10), or GGS (SEQ ID NO: 11).

In various embodiments, the spacer peptide comprises one or more GGGGS (SEQ ID NO: 12) or GGGS (SEQ ID NO: 16) amino acid sequences. In various embodiments, the spacer peptide comprises one or more GGGPS (SEQ ID NO: 14) or GGGSP (SEQ ID NO: 15) amino acid sequences. In various embodiments, the spacer peptide comprises one or more AAAAS (SEQ ID NO: 17) or AAAS (SEQ ID NO: 18) amino acid sequences. In various embodiments, the spacer peptide comprises one or more PAPA (SEQ ID NO: 19) or TPAPA (SEQ ID NO: 20) amino acid sequences. In various embodiments, the spacer peptide comprises one or more AAAKE (SEQ ID NO: 21) amino acid sequences. In various embodiments, the spacer peptide comprises one or more GGGGA (SEQ ID NO: 60) amino acid sequences.

In various embodiments, the spacer peptide comprises an amino acid sequence selected from the group consisting of: (GGGGS)n (SEQ ID NOs: 12, 56, 58, 91-94), (GGGGS)n-GGGPS (SEQ ID NOs: 36, 95-100), GAP-(GGGGS)n-GGGPS (SEQ ID NOs: 101-107), GAP-(GGGGS)n-GGGPS-GAP (SEQ ID NOs: 37, 108-113), GAP-(GGGGS)n-GGGPS-(GGGGS)n-GAP (SEQ ID NOs: 114-162), GAP-GGGPS-(GGGGS)n-GAP (SEQ ID NOs: 163-169), GAP-(GGGGS)u-AAAAS-GGGPS-(GGGGS)u-AAAA-GAP (SEQ ID NOs: 170-218), GAP-(GGGGS)n-PAPAP-(Xaa)n-GAP (SEQ ID NOs: 219-267), GAP-(GGGGS)n-PAPAPT-(Xaa)n-GAP (SEQ ID NOs: 268-316), GAP-(GGGGS)n-(Xaa)n-PAPAP-(Xaa)n-(AAAKE)n-(Xaa)n-(GGGGS)n-GAP (SEQ ID NOs: 544-551), (GGGGA)n (SEQ ID NOs: 60, 79, 81, 317-320), (GGGGA)n-GGGPS (SEQ ID NOs: 321-326), GAP-(GGGGA)n-GGGPS (SEQ ID NOs: 327-333), GAP-(GGGGA)n-GGGPS-GAP (SEQ ID NOs: 334-340), GAP-(GGGGA)n-GGGPS-(GGGGA)n-GAP (SEQ ID NOs: 341-389), GAP-GGGPS-(GGGGA)n-GAP (SEQ ID NOs: 390-396), GAP-(GGGGA)n-AAAAS-GGGPS-(GGGGA)n-AAAA-GAP (SEQ ID NOs: 397-445), GAP-(GGGGA)n-PAPAP-(Xaa)n-GAP (SEQ ID NOs: 446-494), GAP-(GGGGA)n-PAPAPT-(Xaa)u-GAP (SEQ ID NOs: 495-543), GAP-(GGGGA)n-(Xaa)n-PAPAP-(Xaa)u-(AAAKE)n-(Xaa)n-(GGGGA)n-GAP (SEQ ID NOs: 552-559); wherein n is 1 to 7. In various embodiments, n is 1 to 4.

In various embodiments, the spacer is selected from the group consisting of

(SEQ ID NO: 22)
EFGGGGSTR,
(SEQ ID NO: 9)
GAP,
(SEQ ID NO: 12)
GGGGS,
(SEQ ID NO: 23)
GPSGSPG,
(SEQ ID NO: 24)
GPSGSPGT,
(SEQ ID NO: 25)
GPSGSPGH,
(SEQ ID NO: 26)
GGGGSGGGGSGGGGSGGGGSGGGPST,
(SEQ ID NO: 27)
GGGGSGGGGSGGGGSGGGGSGGGPSH,
(SEQ ID NO: 28)
GGGGSGGGGSGGGGSGGGGSGGGPSGGGGSGGGPS,
(SEQ ID NO: 29)
GAPGGGGSGGGGSGGGGSGGGGSGGGPSGGGGSGGGPSGAP,
(SEQ ID NO: 30)
GGGGSGGGGSGGGGSGGGGSGGGPSGGGGSGGGGSGGGGSG
GGGSGGGPS,
(SEQ ID NO: 31)
GAPGGGGSGGGGSGGGGSGGGGSGGGPSGGGGSGGGGSGGGG
SGGGGSGGGPSGAP,
(SEQ ID NO: 32)
GGGGSGGGGSGGGGSGGGPSGGGGSGGGGSGGGPS,
(SEQ ID NO: 33)
GAPGGGGSGGGGSGGGGSGGGPSGGGGSGGGGSGGGPSGAP,
(SEQ ID NO: 34)
GGGGSGGGGSGGGGSGGGPSGGGGSGGGGSGGGPSGGGGSGG
GGSGGGPS,
(SEQ ID NO: 35)
GAPGGGGSGGGGSGGGGSGGGPSGGGGSGGGGSGGGPSGGGGS
GGGGSGGGPSGAP,
(SEQ ID NO: 36)
GGGGSGGGGSGGGGSGGGGSGGGPS,
(SEQ ID NO: 37)
GAPGGGGSGGGGSGGGGSGGGGSGGGPSGAP,
(SEQ ID NO: 38)
GGGGSGGGGSAAAASGGGGSGGGPS,
(SEQ ID NO: 39)
GAPGGGGSGGGGSAAAASGGGGSGGGPSGAP,
(SEQ ID NO: 40)
GGGGSGGGGSAAAASGGGGSGGGGSAAAASGGGGSG
GGGSAAAASGGGPS,
(SEQ ID NO: 41)
GAPGGGGSGGGGSAAAASGGGGSGGGGSAAAASGGGGS
GGGGSAAAASGGGPSGAP,
(SEQ ID NO: 42)
GGGGSGGGGSAAAASGGGPSGGGGSAAAASGGGPSGGG
GSAAAASGGGPS,
(SEQ ID NO: 43)
GAPGGGGSGGGGSAAAASGGGPSGGGGSAAAASGGGPS
GGGGSAAAASGGGPSGAP,
(SEQ ID NO: 44)
GGSPAGSPTSTEEGTSESATPESGPGTSTEPSEGSAPG
SPAGSPGPS,
(SEQ ID NO: 45)
GAPGGSPAGSPTSTEEGTSESATPESGPGTSTEPSEGSA
PGSPAGSPGPSGAP,
(SEQ ID NO: 46)
GGSPAGSPTSTEEGTSESATPESGPGTSTEPSEGSAPGS
PAGSPTSTGPS,
(SEQ ID NO: 47)
GAPGGSPAGSPTSTEEGTSESATPESGPGTSTEPSEGSA
PGSPAGSPTSTGPSGAP,
(SEQ ID NO: 48)
GGGSPAPTPTPAPTPAPTPAGGGPS,
(SEQ ID NO: 49)
GAPGGGSPAPTPTPAPTPAPTPAGGGPSGAP,
(SEQ ID NO: 50)
GGGSPAPAPTPAPAPTPAPAGGGPS,
(SEQ ID NO: 51)
GAPGGGSPAPAPTPAPAPTPAPAGGGPSGAP,
(SEQ ID NO: 52)
GGGSAEAAAKEAAAKEAAAKAGGPS,
(SEQ ID NO: 53)
GAPGGGSAEAAAKEAAAKEAAAKAGGPSGAP,
(SEQ ID NO: 54)
GGGSPAEAAAKEAAAKEAAAKEAAAKEAAAKAPSGGG,
(SEQ ID NO: 55)
GAPGGGSPAEAAAKEAAAKEAAAKEAAAKEAAAKAPSGGGGAP,
(SEQ ID NO: 56)
GGGGSGGGGSGGGGS,
(SEQ ID NO: 57)
GAPGGGGSGGGGSGGGGSGAP,
(SEQ ID NO: 58)
GGGGSGGGGSGGGGSGGGGS,
(SEQ ID NO: 59)
GAPGGGGSGGGGSGGGGSGGGGSGAP,
(SEQ ID NO: 60)
GGGGA,
(SEQ ID NO: 61)
GGGGAGGGGAGGGGAGGGGAGGGPST,
(SEQ ID NO: 62)
GGGGAGGGGAGGGGAGGGGAGGGPSH,
(SEQ ID NO: 63)
GGGGAGGGGAGGGGAGGGGAGGGPSGGGGAGGGPS,
(SEQ ID NO: 64)
GAPGGGGAGGGGAGGGGAGGGGAGGGPSGGGGAGGGPSGAP,
(SEQ ID NO: 65)
GGGGAGGGGAGGGGAGGGGAGGGPSGGGGAGGGGAGGGGAGG
GGAGGGPS,
(SEQ ID NO: 66)
GAPGGGGAGGGGAGGGGAGGGGAGGGPSGGGGAGGGGAGGGG
AGGGGAGGGPSGAP,
(SEQ ID NO: 67)
GGGGAGGGGAGGGGAGGGPSGGGGAGGGGAGGGPS,
(SEQ ID NO: 68)
GAPGGGGAGGGGAGGGGAGGGPSGGGGAGGGGAGGGPSGAP,
(SEQ ID NO: 69)
GGGGAGGGGAGGGGAGGGPSGGGGAGGGGAGGGPSGGGGAGG
GGAGGGPS,
(SEQ ID NO: 70)
GAPGGGGAGGGGAGGGGAGGGPSGGGGAGGGGAGGGPSGGGG
AGGGGAGGGPSGAP,
(SEQ ID NO: 71)
GGGGAGGGGAGGGGAGGGGAGGGPS,
(SEQ ID NO: 72)
GAPGGGGAGGGGAGGGGAGGGGAGGGPSGAP,
(SEQ ID NO: 73)
GGGGAGGGGAAAAASGGGGAGGGPS,
(SEQ ID NO: 74)
GAPGGGGAGGGGAAAAASGGGGAGGGPSGAP,
(SEQ ID NO: 75)
GGGGAGGGGAAAAASGGGGAGGGGAAAAASGGGGAGGGGAAA
AASGGGPS,
(SEQ ID NO: 76)
GAPGGGGAGGGGAAAAASGGGGAGGGGAAAAASGGGGAGGGGA
AAAASGGGPSGAP,
(SEQ ID NO: 77)
GGGGAGGGGAAAAASGGGPSGGGGAAAAASGGGPSGGGGAAAA
ASGGGPS,
(SEQ ID NO: 78)
GAPGGGGAGGGGAAAAASGGGPSGGGGAAAAASGGGPSGGGGAA
AAASGGGPSGAP,
(SEQ ID NO: 79)
GGGGAGGGGAGGGGA,
(SEQ ID NO: 80)
GAPGGGGAGGGGAGGGGAGAP,
(SEQ ID NO: 81)
GGGGAGGGGAGGGGAGGGGA,
(SEQ ID NO: 82)
GAPGGGGAGGGGAGGGGAGGGGAGAP,
GGGGAGGGGAGGGGAGGGGAGGGGAGGGGAGGGGAGGGGAGG
GPS,
[or
(SEQ ID NO: 83)
(GGGGA)₈GGGPS],
GGGGAGGGGAGGGGAGGGGAGGGGAGGGGAGGGGAGGGGAGGG
PSH
[or
(SEQ ID NO: 84)
(GGGGA)₈GGGPSH],
GGGGAGGGGAGGGGAGGGGAGGGGAGGGGAGGGGAGGGGAGGGGA
GGGPS
[or
(SEQ ID NO: 85)
(GGGGA)₉GGGPS],
GGGGAGGGGAGGGGAGGGGAGGGGAGGGGAGGGGAGGGGAGGGG
AGGGPSH
[or
(SEQ ID NO: 86)
(GGGGA)₉GGGPSH],
(SEQ ID NO: 87)
GGGGPAPGPGPAPGPAPGPAGGGPS,
(SEQ ID NO: 88)
GAPGGGGPAPGPGPAPGPAPGPAGGGPGGAP,
(SEQ ID NO: 89)
GGGGPAPAPGPAPAPGPAPAGGGPS,
and
(SEQ ID NO: 90)
GAPGGGGPAPAPGPAPAPGPAPAGGGPGGAP.
In various embodiments, the spacer is selected
from the group consisting of
(SEQ ID NO: 36)
GGGGSGGGGSGGGGSGGGGSGGGPS,
(SEQ ID NO: 44)
GGSPAGSPTSTEEGTSESATPESGPGTSTEPSEGSAPGSPAGSPGPS,
(SEQ ID NO: 45)
GAPGGSPAGSPTSTEEGTSESATPESGPGTSTEPSEGSAPGSPA
GSPGPSGAP,
(SEQ ID NO: 46)
GGSPAGSPTSTEEGTSESATPESGPGTSTEPSEGSAPGSPAGSPTSTGPS,
(SEQ ID NO: 47)
GAPGGSPAGSPTSTEEGTSESATPESGPGTSTEPSEGSAPGSPAGSP
TSTGPSGAP,
(SEQ ID NO: 48)
GGGSPAPTPTPAPTPAPTPAGGGPS,
(SEQ ID NO: 49)
GAPGGGSPAPTPTPAPTPAPTPAGGGPSGAP,
(SEQ ID NO: 50)
GGGSPAPAPTPAPAPTPAPAGGGPS,
(SEQ ID NO: 51)
GAPGGGSPAPAPTPAPAPTPAPAGGGPSGAP,
(SEQ ID NO: 52)
GGGSAEAAAKEAAAKEAAAKAGGPS,
(SEQ ID NO: 53)
GAPGGGSAEAAAKEAAAKEAAAKAGGPSGAP,
(SEQ ID NO: 54)
GGGSPAEAAAKEAAAKEAAAKEAAAKEAAAKAPSGGG,
(SEQ ID NO: 55)
GAPGGGSPAEAAAKEAAAKEAAAKEAAAKEAAAKAPSGGGGAP,
and
(SEQ ID NO: 71)
GGGGAGGGGAGGGGAGGGGAGGGPS.

In various embodiments, the spacer is selected from the group consisting of

(SEQ ID NO: 36)
GGGGSGGGGSGGGGSGGGGSGGGPS,
(SEQ ID NO: 47)
GAPGGSPAGSPTSTEEGTSESATPESGPGTSTEPSEG
SAPGSPAGSPTSTGPSGAP,
(SEQ ID NO: 51)
GAPGGGSPAPAPTPAPAPTPAPAGGGPSGAP,
(SEQ ID NO: 55)
GAPGGGSPAEAAAKEAAAKEAAAKEAAAKEAAAKAP
SGGGGAP,
and
(SEQ ID NO: 71)
GGGGAGGGGAGGGGAGGGGAGGGPS

Additional constructs of GILT-tagged alpha-N-acetylglucosaminidase proteins that can be used in the methods and compositions of the present invention were described in detail in U.S. Publication Nos. 20050244400 and 20050281805, the entire disclosures of which is incorporated herein by reference.

### Cells

Any mammalian cell or cell type susceptible to cell culture, and to expression of polypeptides, may be utilized in accordance with the present invention, such as, for example, human embryonic kidney (HEK) 293, Chinese hamster ovary (CHO), monkey kidney (COS), HT1080, C10, HeLa, baby hamster kidney (BHK), 3T3, C127, CV-1, HaK, NS/0, and L-929 cells. Non-limiting examples of mammalian cells that may be used in accordance with the present invention include, but are not limited to, BALB/c mouse myeloma line (NS0/1, ECACC No: 85110503); human retinoblasts (PER.C6 (CruCell, Leiden, The Netherlands)); monkey kidney CVIline transformed by SV40 (COS-7, ATCC CRL 1651); human embryonic kidney line (293 or 293 cells subcloned for growth in suspension culture, Graham et al., J. Gen Virol., 36:59 (1977)); baby hamster kidney cells (BHK, ATCC CCL 10); Chinese hamster ovary cells +1-DHFR (CHO, Urlaub and Chasm, Proc. Natl. Acad. Sci. USA, 77:4216 (1980)); mouse sertoli cells (TM4. Mather. Biol. Reprod., 23:243-251 (1980)); monkey kidney cells (CV1 ATCC CCL 70); African green monkey kidney cells (VERO-76, ATCC CRL-1 587); human cervical carcinoma cells (HeLa, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TRI cells (Mather et al., Annals N.Y. Acad. Sci., 383:44-68 (1982)); MRC 5 cells; FS4 cells; and a human hepatoma line (Hep G2). In some embodiments, the fusion protein of the present invention is produced from CHO cell lines.

The fusion protein of the invention can also be expressed in a variety of non-mammalian host cells such as, for example, insect (e.g., Sf-9, Sf-21, HiS), plant (e.g., Leguminosa, cereal, or tobacco), yeast (e.g., S cerivisae, P. pastoris), prokaryote (e.g., E. Coli, B. subtilis and other Bacillus spp., Pseudomonas spp., Streptomyces spp), or fungus.

In some embodiments, a fusion protein with or without a furin-resistant GILT tag can be produced in furin-deficient cells. As used herein, the term “furin-deficient cells” refers to any cells whose furin protease activity is inhibited, reduced or eliminated. Furin-deficient cells include both mammalian and non-mammalian cells that do not produce furin or produce reduced amount or defective furin protease. Exemplary furin deficient cells that are known and available to the skilled artisan, including but not limited to FD11 cells (Gordon et al (1997) Infection and Immunity 65(8):3370 3375), and those mutant cells described in Moehring and Moehring (1983) Infection and Immunity 41(3):998 1009. Alternatively, a furin deficient cell may be obtained by exposing the above-described mammalian and non-mammalian cells to mutagenesis treatment, e.g., inadiation, ethidium bromide, bromidated uridine (BrdU) and others, preferably chemical mutagenesis, and more preferred ethyl methane sulfonate mutagenesis, recovering the cells which survive the treatment and selecting for those cells which are found to be resistant to the toxicity of Pseudomonas exotoxin A (see Moehring and Moehtin (1983) Infection and Immunity 41(3):998 1009).

In various embodiments, it is contemplated that certain of the targeted therapeutic proteins comprising a spacer as described herein may exhibit increased expression of active protein when expressed recombinantly compared to targeted therapeutic proteins comprising a different spacer peptide. In various embodiments, it is also contemplated that targeted therapeutic proteins described herein may have increased activity compared to other targeted therapeutic proteins herein. It is contemplated that those targeted therapeutic proteins exhibiting increased expression of active protein and/or having increased activity compared to other targeted therapeutic proteins comprising a different spacer peptide are used for further experimentation.

### Administration of Therapeutic Proteins

In accordance of the invention, a therapeutic protein of the invention is typically administered to the individual alone, or in compositions or medicaments comprising the therapeutic protein (e.g., in the manufacture of a medicament for the treatment of the disease), as described herein. The compositions can be formulated with a physiologically acceptable carrier or excipient to prepare a pharmaceutical composition. The carrier and composition can be sterile. The formulation should suit the mode of administration.

Suitable pharmaceutically acceptable carriers include but are not limited to water, salt solutions (e.g., NaCl), saline, buffered saline, alcohols, glycerol, ethanol, gum arabic, vegetable oils, benzyl alcohols, polyethylene glycols, gelatin, carbohydrates such as lactose, amylose or starch, sugars such as mannitol, sucrose, or others, dextrose, magnesium stearate, talc, silicic acid, viscous paraffin, perfume oil, fatty acid esters, hydroxymethylcellulose, polyvinyl pyrolidone, etc., as well as combinations thereof. The pharmaceutical preparations can, if desired, be mixed with auxiliary agents (e.g., lubricants, preservatives, stabilizers, wetting agents, emulsifiers, salts for influencing osmotic pressure, buffers, coloring, flavoring and/or aromatic substances and the like) which do not deleteriously react with the active compounds or interference with their activity. In a preferred embodiment, a water-soluble carrier suitable for intravenous administration is used.

The composition or medicament, if desired, can also contain minor amounts of wetting or emulsifying agents, or pH buffering agents. The composition can be a liquid solution, suspension, emulsion, tablet, pill, capsule, sustained release formulation, or powder. The composition can also be formulated as a suppository, with traditional binders and carriers such as triglycerides. Oral formulation can include standard carriers such as pharmaceutical grades of mannitol, lactose, starch, magnesium stearate, polyvinyl pyrollidone, sodium saccharine, cellulose, magnesium carbonate, etc.

The composition or medicament can be formulated in accordance with the routine procedures as a pharmaceutical composition adapted for administration to human beings. For example, in a preferred embodiment, a composition for intravenous administration typically is a solution in sterile isotonic aqueous buffer. Where necessary, the composition may also include a solubilizing agent and a local anesthetic to ease pain at the site of the injection. Generally, the ingredients are supplied either separately or mixed together in unit dosage form, for example, as a dry lyophilized powder or water free concentrate in a hermetically sealed container such as an ampule or sachette indicating the quantity of active agent. Where the composition is to be administered by infusion, it can be dispensed with an infusion bottle containing sterile pharmaceutical grade water, saline or dextrose/water. Where the composition is administered by injection, an ampule of sterile water for injection or saline can be provided so that the ingredients may be mixed prior to administration.

The therapeutic protein can be formulated as neutral or salt forms. Pharmaceutically acceptable salts include those formed with free amino groups such as those derived from hydrochloric, phosphoric, acetic, oxalic, taTtaric acids, etc., and those formed with free carboxyl groups such as those derived from sodium, potassium, ammonium, calcium, ferric hydroxides, isopropylamine, triethylamine, 2-ethylamino ethanol, histidine, procaine, etc.

A therapeutic protein (or a composition or medicament containing a therapeutic protein) is administered by any appropriate route. In various embodiments, a therapeutic protein is administered intravenously. In other embodiments, a therapeutic protein is administered by direct administration to a target tissue, such as heart or muscle (e.g., intramuscular), or nervous system (e.g., direct injection into the brain; intraventricularly; intrathecally). In various embodiments, a therapeutic protein is administered intrathecally. Alternatively, a therapeutic protein (or a composition or medicament containing a therapeutic protein) can be administered parenterally, transdermally, or transmucosally (e.g., orally or nasally). More than one route can be used concurrently, if desired, e.g., a therapeutic protein is administered intravenously and intrathecally. Concurrent intravenous and intrathecal administration need not be simultaneous, but can be sequential.

A therapeutic protein (or a composition or medicament containing a therapeutic protein) can be administered alone, or in conjunction with other agents, such as antihistamines (e.g., diphenhydramine) or immunosuppressants or other immunotherapeutic agents which counteract anti-GILT-tagged lysosomal enzyme antibodies. The term, “in conjunction with,” indicates that the agent is administered prior to, at about the same time as, or following the therapeutic protein (or a composition or medicament containing the therapeutic protein). For example, the agent can be mixed into a composition containing the therapeutic protein, and thereby administered contemporaneously with the therapeutic protein; alternatively, the agent can be administered contemporaneously, without mixing (e.g., by “piggybacking” delivery of the agent on the intravenous line by which the therapeutic protein is also administered, or vice versa). In another example, the agent can be administered separately (e.g., not admixed), but within a short time frame (e.g., within 24 hours) of administration of the therapeutic protein.

The therapeutic protein (or composition or medicament containing the therapeutic protein) is administered in a therapeutically effective amount (i.e., a dosage amount that, when administered at regular intervals, is sufficient to treat the disease, such as by ameliorating symptoms associated with the disease, preventing or delaying the onset of the disease, and/or also lessening the severity or frequency of symptoms of the disease, as described above). The dose which will be therapeutically effective for the treatment of the disease will depend on the nature and extent of the disease's effects, and can be determined by standard clinical techniques. In addition, in vitro or in vivo assays may optionally be employed to help identify optimal dosage ranges using methods known in the art. The precise dose to be employed will also depend on the route of administration, and the seriousness of the disease, and should be decided according to the judgment of a practitioner and each patient's circumstances. Effective doses may be extrapolated from dose-response curves derived from in vitro or animal model test systems. The therapeutically effective dosage amount can be, for example, about 0.1-1 mg/kg, about 1-5 mg/kg, about 2.5-20 mg/kg, about 5-20 mg/kg, about 20-50 mg/kg, or about 20-100 mg/kg or about 50-200 mg/kg, or about 2.5 to 20 mg/kg of body weight. The effective dose for a particular individual can be varied (e.g., increased or decreased) over time, depending on the needs of the individual. For example, in times of physical illness or stress, or if disease symptoms worsen, the dosage amount can be increased.

The therapeutically effective amount of the therapeutic protein (or composition or medicament containing the therapeutic protein) is administered at regular intervals, depending on the nature and extent of the disease's effects, and on an ongoing basis. Administration at an “interval,” as used herein, indicates that the therapeutically effective amount is administered periodically (as distinguished from a one-time dose). The interval can be determined by standard clinical techniques. In some embodiments, the therapeutic protein is administered bimonthly, monthly, twice monthly, triweekly, biweekly, weekly, twice weekly, thrice weekly, or daily. The administration interval for a single individual need not be a fixed interval, but can be varied over time, depending on the needs of the individual. For example, in times of physical illness or stress, or if disease symptoms worsen, the interval between doses can be decreased.

As used herein, the term “bimonthly” means administration once per two months (i.e., once every two months); the term “monthly” means administration once per month; the term “triweekly” means administration once per three weeks (i.e., once every three weeks); the term “biweekly” means administration once per two weeks (i.e., once every two weeks); the term “weekly” means administration once per week; and the term “daily” means administration once per day.

The disclosure additionally pertains to a pharmaceutical composition comprising a therapeutic protein, as described herein, in a container (e.g., a vial, bottle, bag for intravenous administration, syringe, etc.) with a label containing instructions for administration of the composition for treatment of Mucopolysaccharidosis Type IIIB (Sanfilippo B Syndrome), such as by the methods described herein.

### Intrathecal Administration of the Pharmaceutically Acceptable Formulations

In various embodiments, the enzyme fusion protein is administered by introduction into the central nervous system of the subject, e.g., into the cerebrospinal fluid of the subject. In certain aspects of the invention, the enzyme is introduced intrathecally, e.g., into the lumbar area, or the cisterna magna or intraventricularly (or intracerebroventricularly) into a cerebral ventricle space. Methods of administering a lysosomal enzyme intrathecally are described in U.S. Pat. No. 7,442,372, incorporated herein by reference in its entirety.

Those of skill in the art are aware of devices that may be used to effect intrathecal administration of a therapeutic composition. For example, the therapy may be given using an Ommaya reservoir which is in common use for intrathecally administering drugs for meningeal carcinomatosis (Ommaya A K, Lancet 2: 983-84, 1963). More specifically, in this method, a ventricular tube is inserted through a hole formed in the anterior horn and is connected to an Ommaya reservoir installed under the scalp, and the reservoir is subcutaneously punctured to intrathecally deliver the particular enzyme being replaced, which is injected into the reservoir. Other devices for intrathecal administration of therapeutic compositions to an individual are described in U.S. Pat. No. 6,217,552, incorporated herein by reference. Alternatively, the composition may be intrathecally given, for example, by a single injection, or continuous infusion. It should be understood that the dosage treatment may be in the form of a single dose administration or multiple doses.

As used herein, the term “intrathecal administration” is intended to include delivering a pharmaceutical composition directly into the cerebrospinal fluid of a subject, by techniques including lateral cerebroventricular injection (i.e., intracerebroventricularly) through a burrhole or cisternal or lumbar puncture or the like (described in Lazorthes et al. Advances in Drug Delivery Systems and Applications in Neurosurgery, 143-192 and Omaya et al., Cancer Drug Delivery, 1: 169-179, the contents of which are incorporated herein by reference). The term “lumbar region” is intended to include the area between the third and fourth lumbar (lower back) vertebrae and, more inclusively, the L2-S1 region of the spine. The term “cisterna magna” is intended to include access to the space around and below the cerebellum via the opening between the skull and the top of the spine. The term “cerebral ventricle” is intended to include the cavities in the brain that are continuous with the central canal of the spinal cord. Administration of a pharmaceutical composition in accordance with the present invention to any of the above mentioned sites can be achieved by direct injection of the composition or by the use of infusion pumps. For injection, the composition of the invention can be formulated in liquid solutions, preferably in physiologically compatible buffers such as Hank's solution, Ringer's solution or phosphate buffer. In addition, the enzyme may be formulated in solid form and re-dissolved or suspended immediately prior to use. Lyophilized forms are also included. The injection can be, for example, in the form of a bolus injection or continuous infusion (e.g., using infusion pumps) of the enzyme.

In various embodiments of the invention, the enzyme is administered by lateral cerebra ventricular injection into the brain of a subject. The injection can be made, for example, through a burr hole made in the subject's skull. In another embodiment, the enzyme and/or other pharmaceutical formulation is administered through a surgically inserted shunt into the cerebral ventricle of a subject. For example, the injection can be made into the lateral ventricles, which are larger, even though injection into the third and fourth smaller ventricles can also be made.

In various embodiments, the pharmaceutical compositions used in the present invention are administered by injection into the cisterna magna, or lumbar area of a subject. In another embodiment of the method of the invention, the pharmaceutically acceptable formulation provides sustained delivery, e.g., “slow release” of the enzyme or other pharmaceutical composition used in the present invention, to a subject for at least one, two, three, four weeks or longer periods of time after the pharmaceutically acceptable formulation is administered to the subject.

In various embodiments, a therapeutic fusion protein is delivered to one or more surface or shallow tissues of the brain or spinal cord. For example, in various embodiments, a therapeutic fusion protein is delivered to one or more surface or shallow tissues of the cerebrum or spinal cord. In some embodiments, the targeted surface or shallow tissues of the cerebrum or spinal cord are located within 4 mm from the surface of the cerebrum. In some embodiments, the targeted surface or shallow tissues of the cerebrum are selected from pia mater tissues, cerebral cortical ribbon tissues, hippocampus, Virchow Robin space, blood vessels within the VR space, the hippocampus, portions of the hypothalamus on the inferior surface of the brain, the optic nerves and tracts, the olfactory bulb and projections, and combinations thereof.

In some embodiments, a therapeutic fusion protein is delivered to one or more deep tissues of the cerebrum or spinal cord. In some embodiments, the targeted surface or shallow tissues of the cerebrum or spinal cord are located 4 mm (e.g., 5 mm, 6 mm, 7 mm, 8 mm, 9 mm, or 10 mm) below (or internal to) the surface of the cerebrum. In some embodiments, targeted deep tissues of the cerebrum include the cerebral cortical ribbon. In some embodiments, targeted deep tissues of the cerebrum include one or more of the diencephalon (e.g., the hypothalamus, thalamus, prethalamus, subthalamus, etc.), metencephalon, lentiform nuclei, the basal ganglia, caudate, putamen, amygdala, globus pallidus, and combinations thereof.

In various embodiments, a targeted surface or shallow tissue of the spinal cord contains pia matter and/or the tracts of white matter. In various embodiments, a targeted deep tissue of the spinal cord contains spinal cord grey matter and/or ependymal cells. In some embodiments, a therapeutic fusion protein is delivered to neurons of the spinal cord.

In various embodiments, a therapeutic fusion protein is delivered to one or more tissues of the cerebellum. In certain embodiments, the targeted one or more tissues of the cerebellum are selected from the group consisting of tissues of the molecular layer, tissues of the Purkinje cell layer, tissues of the Granular cell layer, cerebellar peduncles, and combination thereof. In some embodiments, therapeutic agents (e.g., enzymes) are delivered to one or more deep tissues of the cerebellum including, but not limited to, tissues of the Purkinje cell layer, tissues of the Granular cell layer, deep cerebellar white matter tissue (e.g., deep relative to the Granular cell layer), and deep cerebellar nuclei tissue.

In various embodiments, a therapeutic fusion protein is delivered to one or more tissues of the brainstem. In some embodiments, the targeted one or more tissues of the brainstem include brain stem white matter tissue and/or brain stem nuclei tissue.

In various embodiments, a therapeutic fusion protein is delivered to various brain tissues including, but not limited to, gray matter, white matter, periventricular areas, pia-arachnoid, meninges, neocortex, cerebellum, deep tissues in cerebral cortex, molecular layer, caudate/putamen region, midbrain, deep regions of the pons or medulla, and combinations thereof.

In various embodiments, a therapeutic fusion protein is delivered to various cells in the brain including, but not limited to, neurons, glial cells, perivascular cells and/or meningeal cells. In some embodiments, a therapeutic protein is delivered to oligodendrocytes of deep white matter.

### Kits for Use in the Methods of the Invention

The agents utilized in the methods of the invention may be provided in a kit, which kit may further include instructions for use. Such a kit will comprise a fusion protein as described herein comprising an enzyme for use in the treatment of a lysosomal storage disease and a lysosomal targeting moiety, usually in a dose and form suitable for administration to the host. In various embodiments, the kit will usually comprise a device for delivering the enzyme intrathecally.

A kit may also be provided for the conjugation of an antigen, particularly a polypeptide antigen, to a high uptake moiety, in order to generate a therapeutic composition. For example, a moiety such as an IGF-II mutein, either conjugated to a linker suitable for linking polypeptides, as described above, may be provided. The high uptake moiety may also be provided in an unconjugated form, in combination with a suitable linker, and instructions for use.

Another kit may comprise instructions for the intrathecal administration of the therapeutic compositions of the present invention, in addition to the therapeutic compositions. In certain embodiments, the kits of the invention may comprise catheters or other devices for the intrathecal administration of the enzyme replacement therapy that are preloaded with the therapeutic compositions of the present invention. For example, catheters preloaded with 0.001-0.01 mg, 0.01-0.1 mg, 0.1-1.0 mg, 1.0-10 mg, 10-100 mg, or more of a therapeutic fusion protein comprising a lysosomal enzyme and lysosomal targeting moiety, such as Naglu and IGF-II mutein, in a pharmaceutically acceptable formulation are specifically contemplated. Exemplary catheters may single use catheters that can be discarded after use. Alternatively, the preloaded catheters may be refillable and presented in kits that have appropriate amounts of the enzyme for refilling such catheters.

The invention will be further and more specifically described by the following examples. Examples, however, are included for illustration purposes, not for limitation.

### Example 1—Generation of Spacer Sequences

Lysosomal enzymes comprising GILT tags and spacers have been disclosed in US Patent Publication Nos. 20030082176, 20040006008, 20040005309, and 20050281805. Alpha-N-acetylglucosaminidase (Naglu) fusion proteins comprising spacer peptides are disclosed in US Patent Publication No. 201120232021. Additional spacer peptides for use in targeted therapeutic fusion proteins comprising a lysosomal enzyme and a GILT tag were developed as described below.

Spacers can be developed to link both IGF-II muteins and furin-resistant IGF-II muteins. Exemplary spacers include the following amino acid sequences: EFGGGGSTR (SEQ ID NO: 22) GAP (SEQ ID NO: 9), GGGGS (SEQ ID NO: 12), GGGGA (SEQ ID NO: 60), GPSGSPG (SEQ ID NO: 23), GPSGSPGT (SEQ ID NO: 24), GPSGSPH (SEQ ID NO: 25), GGGGSGGGGSGGGGSGGGGSGGGPS (SEQ ID NO: 36), GGGGAGGGGAGGGGAGGGGAGGGPS (SEQ ID NO: 71), GGGGSGGGGSGGGGSGGGGSGGGPST (SEQ ID NO: 26), GGGGSGGGGSGGGGSGGGGSGGGPSH (SEQ ID NO: 27), and GGGGAGGGGAGGGGAGGGGAGGGPSH (SEQ ID NO: 62).

Constructs comprising a spacer, full-length Naglu (including the signal sequence) and an IGF-II peptide were generated in which the spacer sequence (EFGGGGSTR spacer (SEQ ID NO: 22), GAP spacer (SEQ ID NO: 9), GGGGS spacer (SEQ ID NO: 12), GPSGSPG spacer (SEQ ID NO: 23), or GGGGSGGGGSGGGGSGGGGSGGGPS spacer (SEQ ID NO: 36)) was inserted between full-length Naglu and IGF2 8-67 R37A (SEQ ID NOs: 560-564).

Additional linkers were made based on the XTEN method as described in Schellenberger et al. (Nat Biotech 27:1186-1190, 2009). XTEN-like linkers may provide a longer half-life for the generated fusion protein as compared to other linkers. Exemplary spacers have the amino acid sequences GGSPAGSPTSTEEGTSESATPESGPGTSTEPSEGSAPGSPAGSPGPS (SEQ ID NO: 44), GAPGGSPAGSPTSTEEGTSESATPESGPGTSTEPSEGSAPGSPAGSPGPSGAP (SEQ ID NO: 45), GGSPAGSPTSTEEGTSESATPESGPGTSTEPSEGSAPGSPAGSPTSTGPS (SEQ ID NO: 46), and GAPGGSPAGSPTSTEEGTSESATPESGPGTSTEPSEGSAPGSPAGSPTSTGPSGAP (SEQ ID NO: 47). The spacer can be inserted between Naglu and IGF-2 mutein, optionally via the Asci sites on the constructs.

Protein expression has been associated with the DNA codon used to encode a particular amino acid, e.g., changing the codon for an amino acid can increase expression of the protein without changing the amino acid sequence of the protein (Trinh et al, Mol. Immunol 40:717-722, 2004). Altering the codon encoding the peptide resulted in increased levels of recombinant fusion protein production. Using this technique additional spacer sequences were developed for use in the therapeutic fusion protein with lysosomal enzyme, such as GGGGSGGGGSGGGGS (SEQ ID NO: 56), GAPGGGGSGGGGSGGGGSGAP (SEQ ID NO: 57), GGGGSGGGGSGGGGSGGGGS (SEQ ID NO: 58) and GAPGGGGSGGGGSGGGGSGGGGSGAP (SEQ ID NO: 59). Additional spacer sequences are GGGGAGGGGAGGGGA (SEQ ID NO: 79), GAPGGGGAGGGGAGGGGAGAP (SEQ ID NO: 80), GGGGAGGGGAGGGGAGGGGA (SEQ ID NO: 81) and GAPGGGGAGGGGAGGGGAGGGGAGAP (SEQ ID NO: 82). Any one of these spacers is inserted between Naglu and IGF-11 mutein, optionally via the Asci sites on the constructs.

An exemplary rigid linker which comprises multiple pralines to contribute to rigidity, has the following sequence GGGSPAPTPTPAPTPAPTPAGGGPS (SEQ ID NO: 48), GAPGGGSPAPTPTPAPTPAPTPAGGGPSGAP (SEQ ID NO: 49), GGGSPAPAPTPAPAPTPAPAGGGPS (SEQ ID NO: 50), or GAPGGGSPAPAPTPAPAPTPAPAGGGPSGAP (SEQ ID NO: 51), whereas an exemplary helical linker has the following sequence GGGSAEAAAKEAAAKEAAAKAGGPS (SEQ ID NO: 52), GAPGGGSAEAAAKEAAAKEAAAKAGGPSGAP (SEQ ID NO: 53), GGGSPAEAAAKEAAAKEAAAKEAAAKEAAAKAPSGGG (SEQ ID NO: 54), or GAPGGGSPAEAAAKEAAAKEAAAKEAAAKEAAAKAPSGGGGAP (SEQ ID NO: 55). Any one of these spacers is inserted between Naglu and IGF-II mutein, optionally via the Asci sites on the constructs.

Additional spacers can be generated using codon optimization using technology developed by DNA 2.0 (Menlo Park, CA). The spacers contemplated include

(SEQ ID NO: 32)
GGGGSGGGGSGGGGSGGGPSGGGGSGGGGSGGGPS,
(SEQ ID NO: 33)
GAPGGGGSGGGGSGGGGSGGGPSGGGGSGGGGSGGGPSGAP,
(SEQ ID NO: 28)
GGGGSGGGGSGGGGSGGGGSGGGPSGGGGSGGGPS,
(SEQ ID NO: 29)
GAPGGGGSGGGGSGGGGSGGGGSGGGPSGGGGSGGGPSGAP,
(SEQ ID NO: 30)
GGGGSGGGGSGGGGSGGGGSGGGPSGGGGSGGGGSGGGGSGGGGSGGGPS,
(SEQ ID NO: 31)
GAPGGGGSGGGGSGGGGSGGGGSGGGPSGGGGSGGGGSGGGGSGGGGSGGGPSGAP,
(SEQ ID NO: 34)
GGGGSGGGGSGGGGSGGGPSGGGGSGGGGSGGGPSGGGGSGGGGSGGGPS,
(SEQ ID NO: 35)
GAPGGGGSGGGGSGGGGSGGGPSGGGGSGGGGSGGGPSGGGGSGGGGSGGGPSGAP,
(SEQ ID NO: 36)
GGGGSGGGGSGGGGSGGGGSGGGPS,
(SEQ ID NO: 37)
GAPGGGGSGGGGSGGGGSGGGGSGGGPSGAP,
(SEQ ID NO: 38)
GGGGSGGGGSAAAASGGGGSGGGPS,
(SEQ ID NO: 39)
GAPGGGGSGGGGSAAAASGGGGSGGGPSGAP,
(SEQ ID NO: 40)
GGGGSGGGGSAAAASGGGGSGGGGSAAAASGGGGSGGGGSAAAASGGGPS,
(SEQ ID NO: 41)
GAPGGGGSGGGGSAAAASGGGGSGGGGSAAAASGGGGSGGGGSAAAASGGGPSGAP,
(SEQ ID NO: 42)
GGGGSGGGGSAAAASGGGPSGGGGSAAAASGGGPSGGGGSAAAASGGGPS,
(SEQ ID NO: 43)
GAPGGGGSGGGGSAAAASGGGPSGGGGSAAAASGGGPSGGGGSAAAASGGGPSGAP,
(SEQ ID NO: 67)
GGGGAGGGGAGGGGAGGGPSGGGGAGGGGAGGGPS,
(SEQ ID NO: 68)
GAPGGGGAGGGGAGGGGAGGGPSGGGGAGGGGAGGGPSGAP,
(SEQ ID NO: 63)
GGGGAGGGGAGGGGAGGGGAGGGPSGGGGAGGGPS,
(SEQ ID NO: 64)
GAPGGGGAGGGGAGGGGAGGGGAGGGPSGGGGAGGGPSGAP,
(SEQ ID NO: 65)
GGGGAGGGGAGGGGAGGGGAGGGPSGGGGAGGGGAGGGGAGGGGAGGGPS,
(SEQ ID NO: 66)
GAPGGGGAGGGGAGGGGAGGGGAGGGPSGGGGAGGGGAGGGGAGGGGAGGGPSGAP,
(SEQ ID NO: 69)
GGGGAGGGGAGGGGAGGGPSGGGGAGGGGAGGGPSGGGGAGGGGAGGGPS,
(SEQ ID NO: 70)
GAPGGGGAGGGGAGGGGAGGGPSGGGGAGGGGAGGGPSGGGGAGGGGAGGGPSGAP,
(SEQ ID NO: 71)
GGGGAGGGGAGGGGAGGGGAGGGPS,
(SEQ ID NO: 72)
GAPGGGGAGGGGAGGGGAGGGGAGGGPSGAP,
(SEQ ID NO: 73)
GGGGAGGGGAAAAASGGGGAGGGPS,
(SEQ ID NO: 74)
GAPGGGGAGGGGAAAAASGGGGAGGGPSGAP,
(SEQ ID NO: 75)
GGGGAGGGGAAAAASGGGGAGGGGAAAAASGGGGAGGGGAAAAASGGGPS,
(SEQ ID NO: 76)
GAPGGGGAGGGGAAAAASGGGGAGGGGAAAAASGGGGAGGGGAAAAASGGGPSGAP,
(SEQ ID NO: 77)
GGGGAGGGGAAAAASGGGPSGGGGAAAAASGGGPSGGGGAAAAASGGGPS,
(SEQ ID NO: 78)
GAPGGGGAGGGGAAAAASGGGPSGGGGAAAAASGGGPSGGGGAAAAASGGGPSGAP,
(SEQ ID NO: 87)
GGGGPAPGPGPAPGPAPGPAGGGPS,
(SEQ ID NO: 88)
GAPGGGGPAPGPGPAPGPAPGPAGGGPGGAP,
(SEQ ID NO: 89)
GGGGPAPAPGPAPAPGPAPAGGGPS,
and
(SEQ ID NO: 90)
GAPGGGGPAPAPGPAPAPGPAPAGGGPGGAP.

Any one of these spacers is inserted between Naglu and IGF-II mutein, optionally via the Asci sites on the constructs.

In certain embodiments if the BM-40 extracellular matrix protein signal peptide sequence (Nischt et al., Eur J. Biochem 200:529-536, 1991) is used, the Naglu in the construct does not comprise its own signal peptide sequence. The spacer is inserted between the Naglu sequence and the IGF-II mutein sequence (e.g., IGF2 8-67 R37A). An exemplary BM-40 signal peptide sequence is MRAWIFFLLCLAGRALA (SEQ ID NO: 8). A GAP peptide may be added to the spacer to facilitate cloning and addition of an Asci cloning site. In certain embodiments, if the native Naglu signal peptide sequence (Weber et al., Hum Mol Genet. 5:771-777, 1996) is used, the Naglu is full-length Naglu and the spacer is inserted between the full-length Naglu and the IGF-II mutein sequence (e.g., IGF2 8-67 R37A). A GAP peptide may be added to the spacer to facilitate cloning and addition of an Asci cloning site.

In exemplary constructs, the human Naglu has been “codon optimized” using DNA 2.0 technology. It is contemplated that the Naglu comprises amino acids 1-743 or amino acids 24-743 of human Naglu. In an exemplary construct, the spacer optionally comprises a GAP spacer (Asci restriction enzyme site used for cloning) or any of the following sequences:

(SEQ ID NO: 22)
EFGGGGSTR,
(SEQ ID NO: 9)
GAP,
(SEQ ID NO: 12)
GGGGS,
(SEQ ID NO: 23)
GPSGSPG,
(SEQ ID NO: 24)
GPSGSPGT,
(SEQ ID NO: 25)
GPSGSPGH,
(SEQ ID NO: 26)
GGGGSGGGGSGGGGSGGGGSGGGPST,
(SEQ ID NO: 27)
GGGGSGGGGSGGGGSGGGGSGGGPSH,
(SEQ ID NO: 28)
GGGGSGGGGSGGGGSGGGGSGGGPSGGGGSGGGPS,
(SEQ ID NO: 29)
GAPGGGGSGGGGSGGGGSGGGGSGGGPSGGGGSGGGPSGAP,
(SEQ ID NO: 30)
GGGGSGGGGSGGGGSGGGGSGGGPSGGGGSGGGGSGGGGSGGGGSGGG
PS,
(SEQ ID NO: 31)
GAPGGGGSGGGGSGGGGSGGGGSGGGPSGGGGSGGGGSGGGGSGGGGS
GGGPSGAP,
(SEQ ID NO: 32)
GGGGSGGGGSGGGGSGGGPSGGGGSGGGGSGGGPS,
(SEQ ID NO: 33)
GAPGGGGSGGGGSGGGGSGGGPSGGGGSGGGGSGGGPSGAP,
(SEQ ID NO: 34)
GGGGSGGGGSGGGGSGGGPSGGGGSGGGGSGGGPSGGGGSGGGGSGGG
PS,
(SEQ ID NO: 35)
GAPGGGGSGGGGSGGGGSGGGPSGGGGSGGGGSGGGPSGGGGSGGGGS
GGGPSGAP,
(SEQ ID NO: 36)
GGGGSGGGGSGGGGSGGGGSGGGPS,
(SEQ ID NO: 37)
GAPGGGGSGGGGSGGGGSGGGGSGGGPSGAP,
(SEQ ID NO: 38)
GGGGSGGGGSAAAASGGGGSGGGPS,
(SEQ ID NO: 39)
GAPGGGGSGGGGSAAAASGGGGSGGGPSGAP,
(SEQ ID NO: 40)
GGGGSGGGGSAAAASGGGGSGGGGSAAAASGGGGSGGGGSAAAASGGG
PS,
(SEQ ID NO: 41)
GAPGGGGSGGGGSAAAASGGGGSGGGGSAAAASGGGGSGGGGSAAAAS
GGGPSGAP,
(SEQ ID NO: 42)
GGGGSGGGGSAAAASGGGPSGGGGSAAAASGGGPSGGGGSAAAASGGG
PS,
(SEQ ID NO: 43)
GAPGGGGSGGGGSAAAASGGGPSGGGGSAAAASGGGPSGGGGSAAAAS
GGGPSGAP,
(SEQ ID NO: 44)
GGSPAGSPTSTEEGTSESATPESGPGTSTEPSEGSAPGSPAGSPGPS,
(SEQ ID NO: 45)
GAPGGSPAGSPTSTEEGTSESATPESGPGTSTEPSEGSAPGSPAGSPG
PSGAP,
(SEQ ID NO: 46)
GGSPAGSPTSTEEGTSESATPESGPGTSTEPSEGSAPGSPAGSPTSTG
PS,
(SEQ ID NO: 47)
GAPGGSPAGSPTSTEEGTSESATPESGPGTSTEPSEGSAPGSPAGSPT
STGPSGAP,
(SEQ ID NO: 48)
GGGSPAPTPTPAPTPAPTPAGGGPS,
(SEQ ID NO: 49)
GAPGGGSPAPTPTPAPTPAPTPAGGGPSGAP,
(SEQ ID NO: 50)
GGGSPAPAPTPAPAPTPAPAGGGPS,
(SEQ ID NO: 51)
GAPGGGSPAPAPTPAPAPTPAPAGGGPSGAP,
(SEQ ID NO: 52)
GGGSAEAAAKEAAAKEAAAKAGGPS,
(SEQ ID NO: 53)
GAPGGGSAEAAAKEAAAKEAAAKAGGPSGAP,
(SEQ ID NO: 54)
GGGSPAEAAAKEAAAKEAAAKEAAAKEAAAKAPSGGG,
(SEQ ID NO: 55)
GAPGGGSPAEAAAKEAAAKEAAAKEAAAKEAAAKAPSGGGGAP,
(SEQ ID NO: 56)
GGGGSGGGGSGGGGS,
(SEQ ID NO: 57)
GAPGGGGSGGGGSGGGGSGAP,
(SEQ ID NO: 58)
GGGGSGGGGSGGGGSGGGGS,
(SEQ ID NO: 59)
GAPGGGGSGGGGSGGGGSGGGGSGAP,
(SEQ ID NO: 60)
GGGGA,
(SEQ ID NO: 61)
GGGGAGGGGAGGGGAGGGGAGGGPST,
(SEQ ID NO: 62)
GGGGAGGGGAGGGGAGGGGAGGGPSH,
(SEQ ID NO: 63)
GGGGAGGGGAGGGGAGGGGAGGGPSGGGGAGGGPS,
(SEQ ID NO: 64)
GAPGGGGAGGGGAGGGGAGGGGAGGGPSGGGGAGGGPSGAP,
(SEQ ID NO: 65)
GGGGAGGGGAGGGGAGGGGAGGGPSGGGGAGGGGAGGGGAGGGGAGGG
PS,
(SEQ ID N0: 66)
GAPGGGGAGGGGAGGGGAGGGGAGGGPSGGGGAGGGGAGGGGAGGGGA
GGGPSGAP,
(SEQ ID NO: 67)
GGGGAGGGGAGGGGAGGGPSGGGGAGGGGAGGGPS,
(SEQ ID NO: 68)
GAPGGGGAGGGGAGGGGAGGGPSGGGGAGGGGAGGGPSGAP,
(SEQ ID NO: 69)
GGGGAGGGGAGGGGAGGGPSGGGGAGGGGAGGGPSGGGGAGGGGAGGG
PS,
(SEQ ID NO: 70)
GAPGGGGAGGGGAGGGGAGGGPSGGGGAGGGGAGGGPSGGGGAGGGGA
GGGPSGAP,
(SEQ ID NO: 71)
GGGGAGGGGAGGGGAGGGGAGGGPS,
(SEQ ID NO: 72)
GAPGGGGAGGGGAGGGGAGGGGAGGGPSGAP,
(SEQ ID NO: 73)
GGGGAGGGGAAAAASGGGGAGGGPS,
(SEQ ID NO: 74)
GAPGGGGAGGGGAAAAASGGGGAGGGPSGAP,
(SEQ ID NO: 75)
GGGGAGGGGAAAAASGGGGAGGGGAAAAASGGGGAGGGGAAAAASGGG
PS,
(SEQ ID NO: 76)
GAPGGGGAGGGGAAAAASGGGGAGGGGAAAAASGGGGAGGGGAAAAAS
GGGPSGAP,
(SEQ ID NO: 77)
GGGGAGGGGAAAAASGGGPSGGGGAAAAASGGGPSGGGGAAAAASGGG
PS,
(SEQ ID NO: 78)
GAPGGGGAGGGGAAAAASGGGPSGGGGAAAAASGGGPSGGGGAAAAAS
GGGPSGAP,
(SEQ ID NO: 79)
GGGGAGGGGAGGGGA,
(SEQ ID NO: 80)
GAPGGGGAGGGGAGGGGAGAP,
(SEQ ID NO: 81)
GGGGAGGGGAGGGGAGGGGA,
(SEQ ID NO: 82)
GAPGGGGAGGGGAGGGGAGGGGAGAP,
(SEQ ID NO: 83)
GGGGAGGGGAGGGGAGGGGAGGGGAGGGGAGGGGAGGGGAGGGPS
[or (GGGGA)₈GGGPS],
(SEQ ID NO: 84)
GGGGAGGGGAGGGGAGGGGAGGGGAGGGGAGGGGAGGGGAGGGPSH
[or (GGGGA)₈GGGPSH],
(SEQ ID NO: 85)
GGGGAGGGGAGGGGAGGGGAGGGGAGGGGAGGGGAGGGGAGGGGAGGG
PS
[or (GGGGA)₉GGGPS],
(SEQ ID NO: 86)
GGGGAGGGGAGGGGAGGGGAGGGGAGGGGAGGGGAGGGGAGGGGAGGG
PSH
[or (GGGGA)₉GGGPSH],
(SEQ ID NO: 87)
GGGGPAPGPGPAPGPAPGPAGGGPS,
(SEQ ID NO: 88)
GAPGGGGPAPGPGPAPGPAPGPAGGGPGGAP,
(SEQ ID NO: 89)
GGGGPAPAPGPAPAPGPAPAGGGPS,
and
(SEQ ID NO: 90)
GAPGGGGPAPAPGPAPAPGPAPAGGGPGGAP.

The above spacers are optionally “codon optimized” using DNA 2.0 technology.

Any of the IGF-II muteins described herein, which are optionally “codon optimized” using DNA 2.0 technology, are useful in the present constructs. In exemplary constructs, the IGF-II mutein is a furin-resistant IGF-II mutein, IGF2 8-67 R37A.

### Example 2—Expression and Purification of Constructs

Constructs comprising the above spacers, the Naglu enzyme and the IGF-II targeting peptide are made and recombinantly expressed. In certain embodiments, the constructs comprise a signal peptide. Exemplary signal peptides include, for example and not for limitation, the Naglu signal peptide comprising amino acids 1-23 of full-length Naglu (Weber et al., Hum Mol Genet 5:771-777, 1996) or a signal peptide derived from the BM-40 extracellular matrix protein (Nischt et al., Eur J Biochem 200:529-536, 1991).

DNA encoding the Naglu sequence, the IGF-II mutein and the spacer peptide are inserted into an appropriate expression vector, such as the pEE and pXC GS expression vectors (Lanza Biologics, Berkshire, UK) and the pC3B (BioMarin, in-house) expression vector. An Asci restriction site (ggcgcgcc (SEQ ID NO: 570)) can be inserted into the vector to aid in cloning the therapeutic fusion proteins described herein.

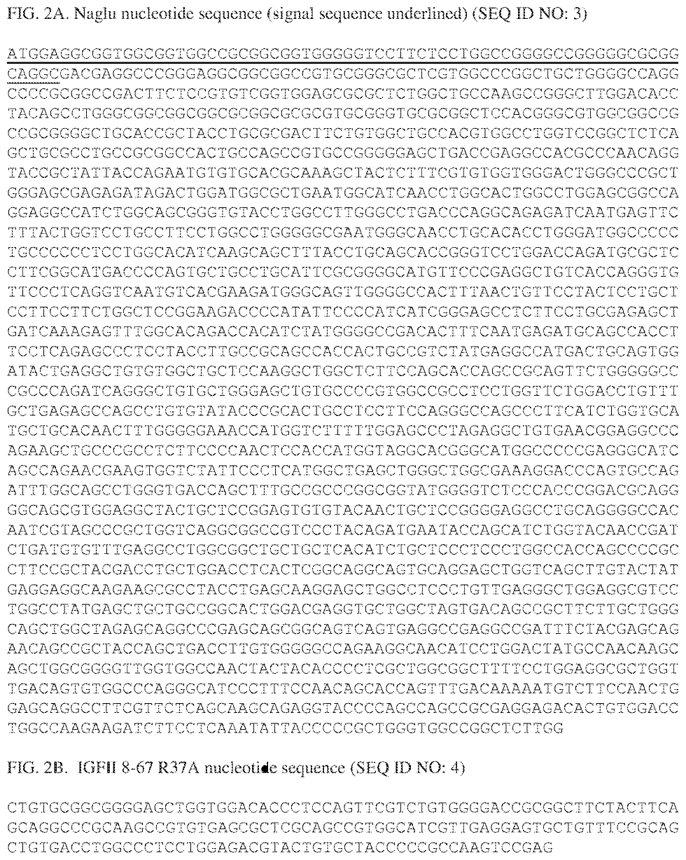

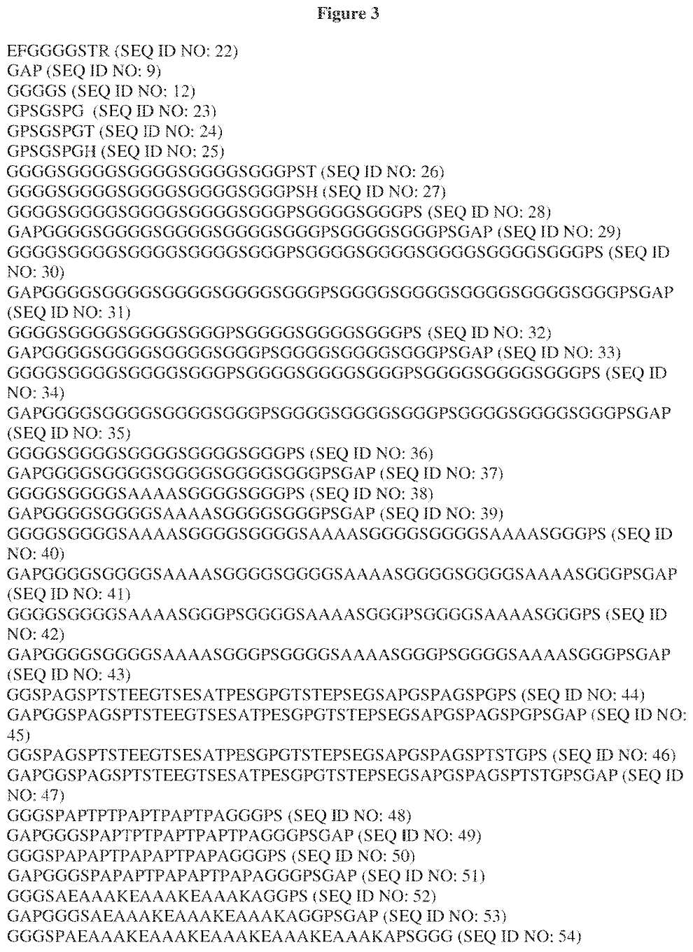

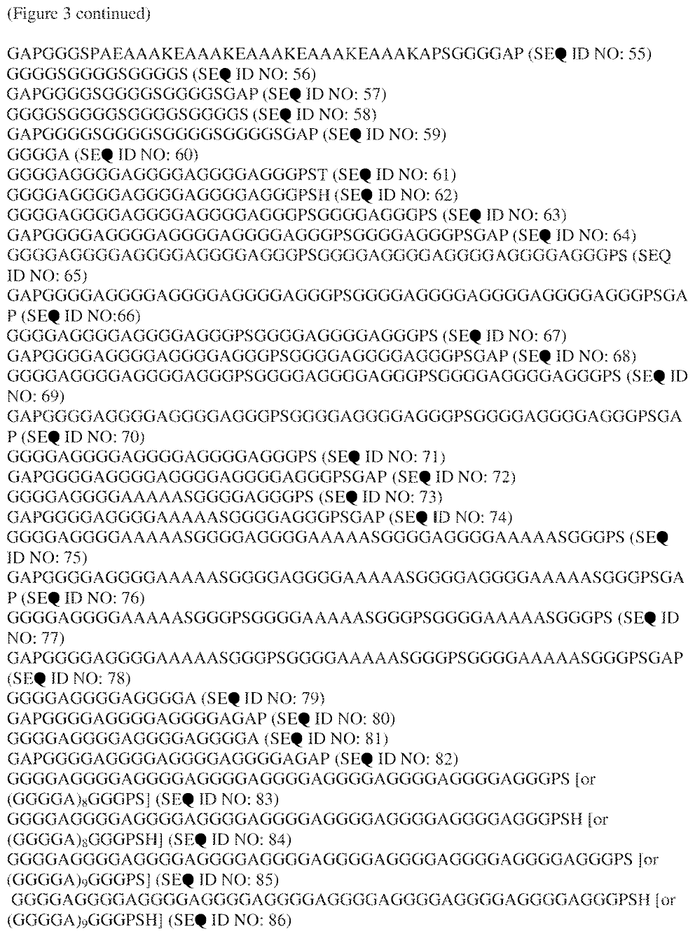

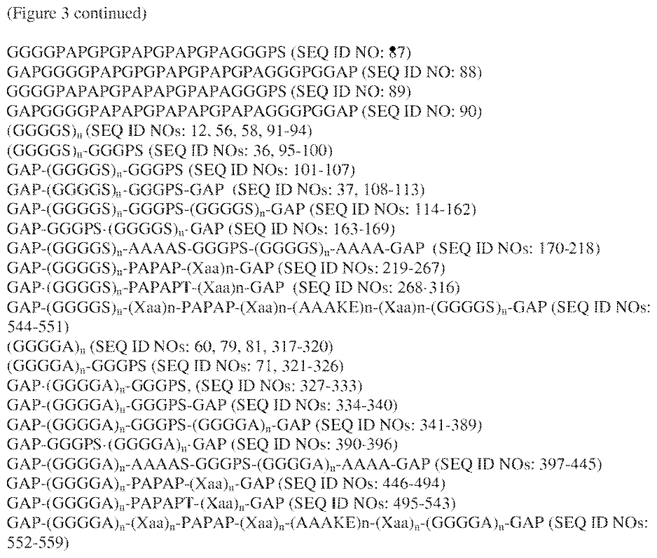

An exemplary construct comprises the full-length Naglu sequence (FIG. 1 and FIG. 2), including signal peptide, a spacer peptide (FIG. 3) and an IGF-II peptide comprising residues 8-67 and having an Ala amino acid substitution at residue Arg-37, R37A (FIGS. 1 and 2), that confers furin resistance to the IGF-II peptide.

Various linker or spacer sequences described in Example 1, connecting the Naglu and IGF-II peptide, are initially evaluated using a transient expression system. GILT-tagged Naglu plasmids (pXC17.4, Lonza) are transfected into suspension CHOKISV GS KO cells (Lonza). 15 μg of plasmid DNA is transfected into 10⁶ cells using electroporation. Media is completely exchanged at 24 hours post-transfection. The transfected cells are seeded in shaker flasks at 1.5×10⁶ cells/ml without selection. Cell growth, viability, titer and specific productivity are determined as the cells are grown at 30° C. for up to 14 days.

GILT-tagged Naglu plasmids are transfected into suspension CHOKISV cells (Lonza). The cells are grown in CDCHO media (Invitrogen) with 6 mM glutamine in shake flasks at 37° C. and 8% C0₂. 30 μg of linearized plasmid DNA in 1×10⁷ cells is transfected into the cells using electroporation. The cells are plated at 5000 cells/well in CDCHO media +40 μM MSX 48 hours after transfection. The plates are incubated at 37° C. and 8% CO₂ for approximately 4-6 weeks to identify clonal growth. The colonies are then screened by the 4 μM activity assay for Naglu (see Example 3) and the highest expressing colonies are transferred to 24 well plates in CDCHO media+40 μM MSX, and then continued to passage the highest expressing clones to 6 well plates, then to shake flasks to identify the highest expressing clones to produce the GILT-tagged Naglu fusion proteins.

Purification is carried out using standard protein purification techniques. For example, in an exemplary purification method, starting material of mammalian cell culture supernatant, as described above, is thawed from storage at −80° C. The material is adjusted with NaCl to reach a final concentration of 1M, followed by 0.2 m sterile filtration.

The filtered material is loaded onto a butyl hydrophobic interaction column, pre-equilibrated with butyl load buffer (20 mM Tris, 1M NaCl, pH 7.5). The bound materials are eluted with a linear gradient over 10 column volumes, using butyl elution buffer (20 mM Tris, pH 7.5). Samples from the elution peaks are pooled, buffer exchanged into 20 mM Tris, pH 7.5, and loaded onto a Q anion exchange column. Bound proteins are then eluted with a linear gradient (10 column volumes) using Q elution buffer (20 mM Tris, 1M NaCl, pH 7.5). Purified samples are then buffer exchanged using centrifugal spin concentrators and sterile-filtered for storage.

Construction, expression, production, purification and formulation of an exemplary Naglu fusion protein: Naglu-(GGGGS)4GGGPS-IGF-II (SEQ ID NO: 568). A DNA construct encoding Naglu-(GGGGS)4GGGPS-IGF-II (SEQ ID NO: 568) was generated by standard recombinant DNA methods. Naglu corresponds to amino acids 1-743 of full-length human Naglu and IGF-II corresponds to the IGF-II mutein comprising amino acids 8-67 of mature human IGF-II with the R37A amino acid substitution that confers furin-resistance. CHOKISV cells were transfected with the DNA construct, and a stable GILT-tagged Naglu-IGF-II fusion protein expressing clone was isolated as described above.

Cells expressing Naglu-(GGGGS)4GGGPS-IGF-II (SEQ ID NO: 568) were grown in a bioreactor, and the Naglu fusion protein was purified from the culture medium as follows. The harvest was salt-adjusted to 1M NaCl, then loaded onto a Butyl Sepharose 4 FF column. The Naglu fusion protein was salt-eluted from the Butyl Sepharose 4 FF column, collected and dialyzed, and then loaded onto a Heparin Sepharose 6 FF column. The Naglu fusion protein was collected in the flow-through fraction, and loaded onto a Q Sepharose HP column. The Naglu fusion protein was salt-eluted from the Q Sepharose HP column, concentrated, and then polished by preparative Sephacryl S300 size exclusion chromatography.

Using this purification procedure, a highly purified, enzymatically active Naglu fusion protein, Naglu-(GGGGS)4GGGPS-IGF-II (SEQ ID NO: 568), was produced. The purified Naglu fusion protein was formulated at 20 mg/mL in artificial CSF (1 mM sodium phosphate, 148 mM sodium chloride, 3 mM potassium chloride, 0.8 mM magnesium chloride, 1.4 mM calcium chloride, pH 7.2).

Construction, expression, production, purification and formulation of exemplary Naglu fusion proteins. DNA constructs encoding Naglu-(GGGGA)4GGGPS-IGF-II (SEQ ID NO: 569), Naglu-Rigid-IGF-II (SEQ ID NO: 566), Naglu-Helical-IGF-II (SEQ ID NO: 567), and Naglu-XTEN-IGF-II (SEQ ID NO: 565) were generated by standard recombinant DNA methods. Naglu corresponds to amino acids 1-743 of full-length human Naglu and IGF-II corresponds to the IGF-II mutein comprising amino acids 8-67 of mature human IGF-II with the R37A amino acid substitution that confers furin-resistance. Exemplary Rigid, Helical and XTEN linkers are described in Example 1. CHOKISV cells were transfected with the DNA constructs, and stable GILT-tagged Naglu-IGF-II fusion protein expressing clones were isolated as described above.

Cells expressing the Naglu-IGF-II fusion proteins were grown in a bioreactor. In typical fed-batch production runs (10-16 days), Naglu-IGF-II constructs with the various linkers all reached titers above 30 mg/L with high cell viability above 80%.

The Naglu fusion proteins were purified from the culture medium as described above. Using this purification procedure, enzymatically active untagged Naglu and Naglu-IGF-II fusion proteins, such as Naglu-Rigid-IGF-II (SEQ ID NO: 566), Naglu-Helical-IGF-II (SEQ ID NO: 567), Naglu-XTEN-IGF-II (SEQ ID NO: 565) and Naglu-(GGGGA)4GGGPS-IGF-II (SEQ ID NO: 569), were purified to −99% purity, as determined by reverse-phase HPLC. The purified untagged Naglu and Naglu fusion proteins were formulated at 20 mg/mL in artificial CSF (1 mM sodium phosphate, 148 mM sodium chloride, 3 mM potassium chloride, 0.8 mM magnesium chloride, 1.4 mM calcium chloride, pH 7.2).

It is contemplated that fusion proteins as described herein that demonstrate higher levels of recombinant expression of active protein and/or increased enzymatic activity compared to fusion proteins comprising a different spacer peptide may be used in further experimentation, such as activity assays, binding assays, uptake assays and in vivo activity assays as described further below.

### Example 3—Activity Assays

To determine the enzymatic activity of the Naglu fusion proteins, an in vitro Naglu activity assay is c:nTied out using a fluorescent labeled synthetic substrate.

Materials used in the assay include: 4-Methylumbelliferyl-N-acetyl-a-D-glucosaminide (4MU-NaGlu Substrate) (Calbiochem, Cat #474500) prepared to final20 mM concentration in 10% DMSO in the assay buffer (0.2 M Sodium Acetate, with or without 1 mg/ml BSA, and 0.005% Tween 20, pH 4.3-4.8) and stored at −80° C. Stock solution of 4-Methylumbelliferone (4-MU Standard) (Sigma, Cat #M1381) is prepared at 10 mM in DMSO and stored at −20° C. in small aliquots. A rhNaglu-His6 control (0.5 mg/ml, R&D Systems, Cat #7096-GH) is diluted to 10 μg/ml in 25 mM Tris, 125 mM NaCl, 0.001% Tween 20, pH7.5 and stored at −80° C. in small aliquots.

On a clear 96 well dilution plate (Granger), 2× serial dilutions of standards in Dilution Buffer (1×PBS, with or without 1 mg/ml BSA, 0.005% Tween 20, pH 7.4 are used, from 200 μM to 1.563 μM plus one blank. On a clear dilution plate, samples are prepared in several dilutions (in Dilution Buffer) to ensure that they are within the standard curve.

10 μl of standards (200 μM to 1.563 μM), control and working samples are transferred to a black non-treated polystyrene 96 well plate (Costar, Cat #3915). 75 μl of substrate (2 mM) is added to each well, followed by incubation for 30 minutes at 37° C. The reaction is then quenched by addition of 200 μl of stop buffer (0.5 M Glycine/NaOH, pH 10.7). The plates are read on an Ex355 Em460 with 455 cut off on a 96-well fluorescent plate reader.

Using this assay, exemplary Naglu fusion proteins, including Naglu-(GGGGS)4GGGPS-IGF-II (SEQ ID NO: 568), Naglu-(GGGGA)4GGGPS-IGF-II (SEQ ID NO: 569), Naglu-Rigid-IGF-II (SEQ ID NO: 566), Naglu-Helical-IGF-11 (SEQ ID NO: 567), and Naglu-XTEN-IGF-II (SEQ ID NO: 565), were shown to have enzymatic activity in vitro, with specific activities toward the synthetic 4MU-Naglu substrate ranging from −175,000 to −220,000 nmol/hr/mg. The enzymatic activity of the Naglu fusion proteins was comparable to that of the untagged Naglu protein (−190,000 nmol/hr/mg). Enzymatic activity data for exemplary Naglu fusion proteins is provided in Table 1.

TABLE 2
Activity of Naglu Fusion Proteins

| Naglu¹ | Linker² | Sp. Act.³ | IC5o⁴ | _{Kuptake}⁵ | t1!2⁵ |
|---|---|---|---|---|---|
| Untagged Naglu | — | 190,000 | — | — | 9.7 |
| Naglu-(GGGGS)4GGGPS-IGF-II | 36 | 190,000 | 0.27, 0.23 | 5.4 | ND |
| Naglu-(GGGGA)4GGGPS-IGF-II | 71 | 220,000 | 0.36 | 6.3 | ND |
| Naglu-Rigid-IGF-II | 51 | 190,000 | 0.23 | 2.4 | 9.5 |
| Naglu-Helical-IGF-II | 55 | 175,000 | 0.25 | 2.3 | 9.4 |
| Naglu-XTEN-IGF-II | 47 | 170,000 | 0.24 | 3.7 | ND |

¹Untagged Naglu and Naglu fusion proteins were constructed, expressed and punfIed as described in Example 2; exemplary Rigid, Helical and XTEN linkers are described in Example 1
²SEQ ID NO: of linkers in the Naglu fusion proteins tested in Examples 3 to 5
³Specific activity (nmol/hr/mg) for Naglu proteins was measured as described in Example 3
⁴IC₅₀ for Naglu proteins for IGF2R competitive binding was measured as described in Example 4
⁵_{Kuptake} and half life (t_{1!2)} for Naglu proteins in MPS-IIIB fibroblasts were measured as described in Example 5

### Example 4—Binding Assays

Binding assays to determine binding of the Naglu fusion proteins to IGF-1, IGF-II and insulin receptors are carried out generally as described in US 20120213762. Briefly, fusion protein constructs are tested for binding affinity for the insulin receptor in an assay measuring the competition of biotinylated insulin binding to plate-bound insulin. An insulin receptor binding assay is conducted by competing insulin, IGF-11, and fusion protein with biotinylated-insulin binding to the insulin receptor (Insulin-R).

Specifically, white Reacti-Bind plates are coated with Insulin-Rat a concentration of 1 μg/well/100 μl (38.4 nM). The coated plates are incubated over night at room temperature, then washed 3× with washing buffer (300 μl/well). The plates are then blocked with blocking buffer (300 μl/well) for 1 hour. The washing steps are repeated and any trace of solution in the plates taken out. Biotinylated-insulin is mixed at 20 nM with different concentrations of insulin, IGF-II, or fusion protein, by serial dilutions. 100 μl of diluted Insulin, IGF-II, or Naglu fusion protein in 20 nM Insulin-biotin are added into the coated plates and the plates are incubated at room temperature for 2 hours. The plates are then washed 3 times with washing buffer. 100 μl of strepavidin-HRP working solution (50 μl strepavidin-HRP in 10 ml blocking buffer) is added into the plates and the plates are incubated at room temperature for 30 minutes. 100 μl of Elisa-Pico working solution containing Elisa-Pico chemiluminescent substrate is added and the chemiluminescence is measured at 425 nm.

### IGF2R Competitive Binding Assay

To measure the ability of the Naglu fusion protein constructs to bind to the IGF-11 receptor a competitive binding assay is carried out. A fragment of the IGFIIR involved with IGF-II binding (domains 10-13, named protein 1288) is coated onto 96-well plates. Biotinylated IGF-II is incubated with the receptor in the presence of increasing amounts of competitors: either control IGF-II (non-biotinylated), or fusion protein sample (containing an IGF-II-derived GILT epitope tag). Receptor-bound biotinylated IGF-II is detected with streptavidin conjugated to horseradish peroxidase (HRP) and a chemiluminescent HRP substrate. The ability of the fusion protein to inhibit binding of biotinylated IGF-II to the IGFIIR is calculated from inhibition curves and reported as an IC₅₀ value (concentration required to achieve 50% binding inhibition).

For the assay, IGFIIR is coated in a white Reacti-bind plate (Pierce, Cat #437111) at 0.5 μg/well in a volume of 100 μl (69.6 nM/per well) in coating buffer. The plate is sealed and incubated overnight at room temperature. The plate is then washed 3× with wash buffer, blocked with blocking buffer and then washed again 3× with wash buffer (300 l/well).

Next, 8 nM IGF-II-biotin is mixed with different concentrations of competitors (IGF-II (non-biotinylated), Reference Protein, or Naglu fusion protein samples, and added into an IGFIIR-coated plate in 2× serial dilutions.

The plate is incubated at room temperature for 2 hours, followed by washing the plate 3× with wash buffer. Streptavidin-HRP is prepared in blocking buffer (1:200 dilution), and 100 al/well added to the plate. IGF-II-Biotin binding activity is detected via streptavidin-HRP using Pico-Elisa reagents. Briefly, the prepared Pico-Elisa working solution is added per well (100 μl/well), and incubated at room temperature for 5 minutes with gentle rocking, then the chemiluminescence at 425 nm is measured.

The IC₅₀ of the samples are calculated using the percent IGF-II-Biotin Bound for each concentration of inhibitor.

Using this competitive IGFIIR binding assay, an exemplary Naglu fusion proteins, including Naglu-(GGGGS)₄GGGPS-IGF-II (SEQ ID NO: 568), Naglu-(GGGGA)₄GGGPS-IGF-II (SEQ ID NO: 569), Naglu-Rigid-IGF-II (SEQ ID NO: 566), Naglu-Helical-IGF-II (SEQ ID NO: 567), and Naglu-XTEN-IGF-II (SEQ ID NO: 565), were shown to have an ICso value of 0.23-0.36 nM. Untagged Naglu protein had no detectable binding in this assay. IGF2R competitive binding data for exemplary Naglu fusion proteins is provided in Table 1.

### Example 5—Uptake Assays

To measure the ability of a Lysosomal Storage Disease enzyme to enter cells via receptor-mediated endocytosis an uptake assay is carried out which measures enzyme uptake using the CI-MPR receptor in rat myoblast L6 cells or in human MPS IIIB fibrobalsts. Mannose-6-phosphate (M6P) and IGF-II are used as inhibitors to determine the site of binding to the CI-MPR receptor. Data is collected to generate a saturation curve for enzyme uptake and determine the kinetic parameter, Kuptake, of the process.

Prior to the uptake assay (24 hours), L6 cells (L6 Rat Myoblasts, ATCC #CRL-1458) or human MPS IIIB fibroblasts are plated at a density of 1×10⁵ cells per well in 24-well plates (VWR #62406-183) and seeded 0.5 ml per well. On the morning of assay, enzyme is mixed with uptake media (1 L DMEM, 1.5 g Sodium Bicarbonate. 0.5 g Bovine Serum Albumin, 20 ml of L-glutamine (200 mM (Gibco #25030-081)), 20 ml of 1M of HEPES (Gibco #1563080)) (20 mM final), pH 7.2) in a tissue culture hood. Enzyme amounts may range from 2-500 nM. The final volume of uptake media+enzyme is 0.5 ml per well. M6P (5 mM final concentration) and/or IGF-II (2.4 μM or 18 μg/ml final concentration) are added to appropriate samples. For uptake inhibition, 18 μl IGF-II stock (1 mg/ml, 133.9 μM) is added per mL of uptake media.

Growth media is aspirated from cells and 0.450 ml of enzyme in uptake buffer added to each well. Note time and return cells to incubator for 18 hours. Plate is removed from incubator and uptake buffer aspirated off cells. Plates are washed 4× by addition of 0.5 ml Dulbecco's PBS and aspirating off. 200 μl of CelLytic M lysis buffer (Sigma) is added to the plates and shaken at room temperature for 20-30 minutes. Lysate is removed from cells and stored in a tape-covered clear 96-well plate (VWR) at −80° C. until ready to assay.

For the enzyme assay, 5 μl of each lysate is added in duplicate by adding to 15 μl of enzyme reaction mix (e.g., Naglu+4MU assays) in black 96-well plate (VWR) (see above) and enzyme/units/ml/hr determined in each lysate.

For the lysate protein assay, 10 μl of each lysate in duplicate are assayed using a Pierce BCA protein Assay kit according to manufacturer's instructions. To measure absorbance, absorbance is read at 562 nm with a plate reader (BMG FluoStar Optima Plate reader) and ug/ml concentration determined.

For each enzyme load, uptake is units of enzyme activity/mg lysate. To determine uptake, the enzyme units/ml are divided by protein ug/ml and multiplied by 1000 (uptake from blank wells subtracted). Results of the assays with or without inhibitors are compared to determine receptor uptake specificity.

For saturation curves, 10 enzyme load concentrations ranging from 0.2-100 nM are used to generate a saturation curve using the assays described above.

Using this assay, an exemplary Naglu fusion protein, Naglu-(GGGGS)₄GGGPS-IGF-II (SEQ ID NO: 568), was shown to have a Kuptake of 7-9 nM in MPS-IIIB fibroblasts.

Alternatively, prior to the uptake assay (24 hours), L6 cells or human MPS IIIB fibroblasts are plated at a density of 1×10⁵ cells per 0.5 ml per well in the 24-well plates. Enzyme samples at 1.6-50 nM are prepared in uptake media: 1 L DMEM, 1.5 g Sodium Bicarbonate. 0.5 g Bovine Serum Albumin, 20 ml of 200 mM L-glutamine and 20 ml of 1M HEPES, pH7.2. For uptake inhibition, M6P (up to 5.0 mM final) and/or IGF-II (up to 1.0 1-1M final) are added to appropriate samples.

Growth media is aspirated from cells and replaced by 0.5 ml of the enzyme preparation in uptake buffer per well. After 4-hour incubation, plates are washed 2 times with 0.5 ml Dulbecco's PBS. 100 μl of M-PER lysis buffer (Pierce) is added to the plates and shaken at room temperature for 10 minutes. Lysate is stored at −80° C. until ready to assay.

For the enzyme assay, 10 μl of each lysate is added in duplicate to the black 96-well plate (see above).

For the lysate protein assay, 10 μl of each lysate in duplicate are assayed using a Pierce BCA Protein Assay Kit according to manufacturer's instructions. Absorbance is read at 562 nm with a plate reader (BMG FluoStar Optima Plate reader) and g/ml concentration determined using BSA as a standard.

For each enzyme load, uptake is expressed as nmoles of 4-MU liberated in 30 minutes. For saturation curves, enzyme concentrations ranging from 1.6-50 nM are used to generate a saturation curve using the assays described above.

Cellular stability of the Naglu fusion proteins was determined by monitoring intracellular Naglu activity over the period of −8 days. Human MPS IIIB fibroblasts plated at a density of 1×10⁵ cells per well in 24-well plates (VWR #62406-183) were treated with Naglu fusion protein at 20 nM final concentration for 4 hours. After 4-hour incubation, cells were switched to growth media without Naglu fusion protein. For each time point (4 hours, 28 hours, 4 days, 6 days & 8 days), cells were lysed in 100 μl of M-PER lysis buffer (Pierce) at room temperature for 10 minutes, and assayed for enzyme activity using a 4-MU labeled substrate. Reduction of Naglu activity over the 8-day sample period can be fit to first-order kinetics to approximate a cellular half-life of the protein.

Using this assay, exemplary Naglu fusion proteins, including Naglu-(GGGGS)4GGGPS-IGF-II (SEQ ID NO: 568), Naglu-(GGGGA)4GGGPS-IGF-II (SEQ ID NO: 569), Naglu-Rigid-IGF-II (SEQ ID NO: 566), Naglu-Helical-IGF-II (SEQ ID NO: 567), and Naglu-XTEN-IGF-II (SEQ ID NO: 565), were shown to be internalized into MPS IIIB fibroblasts with Kuptake values of −2.3-6.3 nM. Untagged Naglu protein, in contrast, was not taken up by the cells under these experimental conditions. Furthermore, the observed uptake of Naglu fusion protein was inhibited by IGF-II, but not by M6P. After uptake, exemplary Naglu fusion proteins were found to be stable with an estimated half-life of −9.5 days, based on enzymatic activity (4-MU substrate) measured in cell lysates. Uptake and half-life data for exemplary Naglu fusion proteins is provided in Table 1.

### Example 6—In Vivo Naglu Fusion Protein Activity

To determine the activity of Naglu fusion proteins in vivo, the fusion proteins are administered to Naglu knock-out animals (see Li et al., Proc Natl Acad Sci USA 96:14505-510, 1999). Naglu knockouts present with large amounts of heparan sulfate in the brain, liver and kidney, increase of beta-hexosaminidase activity and lysosomal-associated membrane protein 2 (LAMP-2) staining in brain, and elevation of gangliosides in brain.

Activity and biodistribution of the exogenous enzyme are determined after 4 ICV (intracerebroventricular) injections over a two week period (100 μg/injection) of recombinant human (rh) Naglu-IGF2. A permanent cannulae is implanted in the mouse (n=12/gp, 8-12 wks old at start) and adjusted to cover those mice whose cannulae are not in the ventricle. Endpoint measurements include Naglu biodistribution, reduction of GAG, e.g., heparan sulfate, storage in the lysosomes of brain cells, and activation of astrocytes and microglia. Levels of various lysosomal or neuronal biomarkers (Ohmi et al., PLoS One 6:e27461, 2011) measured in treated and control groups levels include, but are not limited to, Lysosomal-associated membrane protein 1 (LAMP-1), LAMP-2, glypican 5, Naglu-specific non-reducing ends (NREs) of heparan sulfate, gangliosides, cholesterol, Subunit c of Mitochondrial ATP Synthase (SCMAS), ubiquitin, P-GSK3beta, beta amyloid, P-tau (phosphorylated-Tau), GFAP (astrocyte activation) and CD68 (microglial activation).

Additional experiments to determine survival and behavioral analysis are carried out using mice receiving 4 ICV injections over a two week period of rhNaglu-IGF2 (n=12/gp, 5 months old at stmi, 100 J.lglinjection). Endpoints to be measured include survival time, open field activity, Naglu biodistribution, reduction of GAG, e.g., heparan sulfate, storage in lysosomes, levels of lysosomal or neuronal biomarkers, such as LAMP-1, LAMP-2, glypican 5, gangliosides, cholesterol, SCMAS, ubiquitin, P-GSK3beta, beta amyloid, P-tau, GFAP and CD68.

Naglu knockout mice (Naglu−/−) having a mutation in exon 6 of the naglu gene have been developed (Li et al., Proc Natl Acad Sci US A. 96:14505-10, 1999). The exon 6 site was chosen because this is the site of many mutations in humans. No Naglu activity is detected in homozygous mice, and there is reduced Naglu activity in heterozygotes. Naglu−/− mice have reduced survival times (6-12 months), and may have other functional phenotypes like reduced activity levels. The effects of Naglu fusion proteins on the Naglu−/− mice are assayed.

Naglu−/− mice (n=8) and 8 vehicle control Naglu−/− mice (n=8 littermate heterozygotes) are administered 4 rev doses (100 μg Naglu-rGF2/dose) over 2 weeks. At day −2, mice are anesthetized and the left lateral ventricle cannulated. The mice are allowed to recover. At days 1, 5, 10 and 14, mice are anesthetized (Benedryl, 5 mg/kg rP) 15 minutes prior to rev dose. The rev dose is infused via cannula, 5 Jll volume over 15 minutes, and mice are allowed to recover. On day 15, mice are sacrificed, exsanguinated and serum frozen. Brains are harvested and rR dye is injected into the cannula and the cannula imaged.

The following assays are carried out to determine the effects of Naglu fusion proteins: body weight assessment, NIR imaging for cannula placement, assessment of Naglu-rGF2, GFAP, LAMP-1 and LAMP-2 levels in brain using immunohistochemistry, biochemical assay for Naglu activity, f3-hexosaminadase levels and activity, SensiPro assay to detect non-reducing ends of accumulated glycosaminoglycans (GAGs) specific for Mucopolysaccharidosis IIIb (MPS-IIIb) (WO 2010/078511A2), GM3 ganglioside levels as measured by biochemical assay, as well as immunostain for SeMAS, A-beta, glypican 5, eD68, GFAP and Naglu in medial entorrhinal cortex (Li et al., supra).

Effective treatment with Naglu-rGF2 is expected to result in a decrease in levels of LAMP-1, LAMP-2, GFAP, eD68, SeMAS, A-beta, glypican 5, f3-hexosaminadase, GM3 ganglioside, and MPS-IIrb-specific GAGs.

In vivo efficacy of exemplary Naglu fusion proteins in a mouse model of MPS IIIb. Four rev doses (100 μg/dose) of Naglu-rGF-II fusion protein, either Naglu-(GGGGS)4GGGPS-rGF-II (SEQ ID NO: 568) or Naglu-Rigid-rGF-II (SEQ ID NO: 566), were administered over a two week period to Naglu−/− mice (n=8). Naglu−/− mice (n=8) and eight heterozygous or wild-type littermates (n=8) were given vehicle alone as a control. At day −5, mice were anesthetized; the left lateral ventricle of the brain was cannulated. The mice were allowed to recover. On days 1, 5, 10 and 14, mice were anesthetized with inhaled isoflourane. Benadryl (5 mg/kg rP) was administered to each mouse 15 minutes prior to rev dose to reduce any potential histamine release in response to the Naglu-rGF-II treatment. The rev dose was infused via the implanted cannula, 5 μl volume over 15-20 minutes, and the mice were allowed to recover. At 1, 7, 14, and 28 days following the final dose, mice were sacrificed. Brains were harvested and divided sagittally into 5 sections for distribution to various assays.

The following assays were carried out to determine the effects of Naglu-IGF-II fusion protein: immunohisochemical assessment of Naglu, LAMP-2, GFAP and CD68 levels in brain, biochemical assays for Naglu and beta-hexosaminadase activity, SensiPro assay (Deakin et al., Glycobiology 18:483, 2008; Lawrence et al., Nat Chem Biol. 8:197, 2012; Lawrence et al., J Biol Chem. 283:33674, 2008) to detect total heparan sulfate and NREs of heparan sulfate specific for Mucopolysaccharidosis IIIB (MPS-IIIB) (WO 2010/078511A2), and immunofluorescent staining for SCMAS, beta-amyloid (A-beta), p-Tau, P-GSK3beta, glypican 5, GFAP and CD68 in medial entorrhinal cortex (Li et al., supra).

When evaluated 24 hours after the final dose, treatment with either Naglu-(GGGGS)4GGGPS-IGF-II (SEQ ID NO: 568) or Naglu-Rigid-IGF-II (SEQ ID NO: 566) fusion protein resulted in a marked increase in Naglu enzyme activity, with a concomitant decrease in beta-hexosaminadase activity and levels of total heparan sulfate, Naglu-specific NREs of heparan sulfate, and LAMP-2. Naglu enzyme was easily detectable in brain tissues, not only in cortex, hippocampus, dentate gyrus and thalamus, but also in remote distal geographic locations, including amygdyla, perirhinal cortex and hypothalamus. Significant decreases in the levels of CD68, SCMAS, beta-amyloid (A-beta), p-Tau, P-GSK3beta, and glypican 5 were also observed in Naglu−/− brains upon treatment with Naglu-IGF-11. GFAP staining did not change by 24 hours post-last-dose. Immunohistochemistry demonstrated the presence of Naglu enzyme in many areas of the brain, inside neurons and glial cells, co-localizing with LAMP-2.

Levels of heparan sulfate, Naglu-specific NREs, and beta-hexosaminidase activity continued to decrease over the 7, 14, and 28 days-post-last-dose timepoints. At 28 days, all analytes were at or near the normal mouse control values.

### EQUIVALENTS

Those skilled in the art will recognize, or be able to ascertain using no more than routine experimentation, many equivalents to the specific embodiments of the invention described herein. The scope of the present invention is not intended to be limited to the above Description, but rather is as set forth in the appended claims. The articles “a”, “an”, and “the” as used herein in the specification and in the claims, unless clearly indicated to the contrary, should be understood to include the plural referents. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention also includes embodiments in which more than one, or all, of the group members are present in, employed in, or otherwise relevant to a given product or process. Furthermore, it is to be understood that the invention encompasses variations, combinations, and permutations in which one or more limitations, elements, clauses, descriptive terms, etc., from one or more of the claims is introduced into another claim dependent on the same base claim (or, as relevant, any other claim) unless otherwise indicated or unless it would be evident to one of ordinary skill in the art that a contradiction or inconsistency would arise. Where elements are presented as lists, e.g., in Markush group or similar format, it is to be understood that each subgroup of the elements is also disclosed, and any element(s) can be removed from the group. It should it be understood that, in general, where the invention, or aspects of the invention, is/are referred to as comprising particular elements, features, etc., certain embodiments of the invention or aspects of the invention consist, or consist essentially of, such elements, features, etc. For purposes of simplicity those embodiments have not in every case been specifically set forth herein. It should also be understood that any embodiment of the invention, e.g., any embodiment found within the prior art, can be explicitly excluded from the claims, regardless of whether the specific exclusion is recited in the specification.

It should also be understood that, unless clearly indicated to the contrary, in any methods claimed herein that include more than one act, the order of the acts of the method is not necessarily limited to the order in which the acts of the method are recited, but the invention includes embodiments in which the order is so limited. Furthermore, where the claims recite a composition, the invention encompasses methods of using the composition and methods of making the composition. Where the claims recite a composition, it should be understood that the invention encompasses methods of using the composition and methods of making the composition.

All publications and patent documents cited in this application are incorporated by reference in their entirety to the same extent as if the contents of each individual publication or patent document were incorporated herein.

1.-61. (canceled)

62. A targeted therapeutic fusion protein comprising (a) a human α-N-acetylglucosaminidase (Naglu) protein comprising amino acids 1-743 or 24-743 of SEQ ID NO: 1, (b) a peptide tag having an amino acid sequence at least 70% identical to amino acids 8-67 of SEQ ID NO: 5 (mature human IGF-II), and (c) a spacer peptide between the lysosomal enzyme and the peptide tag, wherein the spacer peptide comprises the amino acid sequence of GGSPAGSPTSTEEGTSESATPESGPGTSTEPSEGSAPGSPAGSPGPS (SEQ ID NO: 44).

63. The targeted therapeutic fusion protein of claim 62, wherein the spacer peptide consists of the amino acid sequence of SEQ ID NO: 44.

64. The targeted therapeutic fusion protein of claim 62, wherein the peptide tag is an N-terminal tag or a C-terminal tag.

65. The targeted therapeutic fusion protein of claim 62, wherein the peptide tag comprises amino acids 8-67 of SEQ ID NO: 5.

66. The targeted therapeutic fusion protein of claim 62, wherein the peptide tag comprises amino acids 8-67 of SEQ ID NO: 5 except for a mutation at the amino acid position corresponding to position 37 of SEQ ID NO: 5.

67. The targeted therapeutic fusion protein of claim 66, wherein the mutation is a substitution of alanine for arginine.

68. A pharmaceutical composition comprising the targeted therapeutic fusion protein of claim 62, and a pharmaceutically acceptable carrier, diluent, or excipient.

69. A method for treating Mucopolysaccharidosis Type IIB (Sanfilippo B syndrome) in a subject comprising administering to the subject a therapeutically effective amount of a pharmaceutical composition comprising a fusion protein comprising a human α-N-acetylglucosaminidase (Naglu) protein comprising amino acids 1-743 or 24-743 of SEQ ID NO: 1, a peptide tag having an amino acid sequence at least 70% identical to amino acids 8-67 of SEQ ID NO: 5 (mature human IGF-II), and a spacer peptide between the lysosomal enzyme and the peptide tag, wherein the spacer peptide comprises the amino acid sequence of GGSPAGSPTSTEEGTSESATPESGPGTSTEPSEGSAPGSPAGSPGPS (SEQ ID NO: 44).

70. The method of claim 69, wherein the spacer peptide consists of the amino acid sequence of SEQ ID NO. 44.

71. The method of claim 69, wherein the peptide tag is an N-terminal tag or a C-terminal tag.

72. The method of claim 69, wherein the peptide tag comprises amino acids 8-67 of SEQ ID NO: 5.

73. The method of claim 69, wherein the peptide tag comprises amino acids 8-67 of SEQ ID NO: 5 except for a mutation at the amino acid position corresponding to position 37 of SEQ ID NO: 5.

74. The method of claim 73, wherein the mutation is a substitution of alanine for arginine.

75. The method of claim 69, wherein the fusion protein is administered intrathecally.

76. The method of claim 69, wherein the effective amount of the fusion protein is in the range of 2.5-20 mg per kilogram of body weight of the subject.

77. A nucleic acid encoding a fusion protein comprising (a) a human α-N-acetylglucosaminidase (Naglu) protein comprising amino acids 1-743 or 24-743 of SEQ ID NO: 1, (b) a peptide tag having an amino acid sequence at least 70% identical to amino acids 8-67 of SEQ ID NO: 5 (mature human IGF-II), and (c) a spacer peptide between the lysosomal enzyme and the peptide tag, wherein the spacer peptide comprises the amino acid sequence of GGGGSGGGGSAAAASGGGPSGGGGSAAAASGGGPSGGGGSAAAASGGGPS (SEQ ID NO: 42), GGGGSGGGGSAAAASGGGGSGGGPS (SEQ ID NO: 38), or GGGGAGGGGAAAAASGGGGAGGGPS (SEQ ID NO: 73), or a combination thereof.

78. The nucleic acid of claim 77, wherein the peptide tag comprises amino acids 8-67 of SEQ ID NO: 5.

79. The nucleic acid of claim 77, wherein the peptide tag comprises amino acids 8-67 of SEQ ID NO: 5 except for a mutation at the amino acid position corresponding to position 37 of SEQ ID NO: 5.

80. The nucleic acid of claim 79, wherein the mutation is a substitution of alanine for arginine.

81. A cell containing the nucleic acid of claim 77.
